(19) United States
(12) Patent Application Publication
(10) Pub. No.: US 2021/0214359 A1
(43) Pub. Date: Jul. 15, 2021

(54) Compounds and Methods for Treating Bacterial Infections

(71) Applicant: NEW YORK UNIVERSITY, New York, NY (US)

(72) Inventors: Evgeny NUDLER, New York, NY (US); Konstantin SHATALIN, Springfield, NJ (US); Dmitrii SHISHOV, Verkhovazhye (RU); Peter FEDICHEV, Dolgoprudny (RU)

(21) Appl. No.: 17/059,464

(22) PCT Filed: May 29, 2019
(86) PCT No.: PCT/US2019/034444

§ 371 Date: Nov. 29, 2020

Related U.S. Application Data
(60) Provisional application No. 62/677,491, filed on May 29, 2018.

(51) Int. Cl. C07D 487/04; C07D 409/04; C07D 413/06; C07D 403/06; C07D 231/56; C07D 209/12; C07D 403/04; C07D 405/12; C07D 413/04; C07D 403/10; C07D 417/10; C07D 401/14; C07D 495/04; C07D 471/04; A61K 45/06

(52) U.S. Cl. CPC C07D 487/04; A61K 45/06; C07D 413/06; C07D 403/06; C07D 231/56; C07D 209/12; C07D 403/04; C07D 405/12; C07D 413/04; C07D 403/10; C07D 417/10; C07D 401/14; C07D 495/04; C07D 471/04; C07D 409/04

(57) ABSTRACT

This invention relates to compounds, pharmaceutical compositions comprising them, and methods of using the compounds and compositions for treating bacterial infections. This invention relates more particularly to compounds and pharmaceutical compositions thereof, methods of inhibiting H₂S-producing enzymes with the compounds, and methods of treating bacterial infections with the compounds.

### CROSS-REFERENCE TO RELATED APPLICATIONS

This application claims the benefit of priority of U.S. Provisional Patent Application No. 62/677,491, filed May 22, 2019, which is incorporated by reference in its entirety.

### BACKGROUND OF DISCLOSURE

### Field of Invention

This invention relates to compounds, pharmaceutical compositions comprising them, and methods of using the compounds and compositions for treating bacterial infections. More particularly, this invention relates to compounds and pharmaceutical compositions thereof, methods of inhibiting H₂S-producing enzymes with the compounds, and methods of treating bacterial infections with the compounds.

### Technical Background

Despite the phenomenal success of antibiotics, infectious diseases remain the second leading cause of death worldwide. About two million Americans are infected in hospitals each year (with 90,000 deaths as a result), and more than half of these infections resist at least one antibiotic. For example, pathogens can alarmingly become fully resistant to last resort antibiotics, such as vancomycin. The emergence of multidrug-resistant bacteria has created a situation in which there are few or no options for treating certain infections. Natural antibiotics and their derivatives are intrinsically prone to become obsolete because of preexisting genes that render pathogens resistant to them. Bacterial species share these genes, thus rapidly spreading resistance from hospitals and farms to surrounding communities.

S. aureus is a major challenge for antibiotic treatment therapies. This bacterium colonizes the skin, nares and gastrointestinal tract of humans in approximately 50% of the human population and causes invasive disease, skin and soft tissue infections, bacteremia, sepsis, pneumonia, osteomyelitis and endocarditis. In humans, S. aureus infection does not usually induce strong immune response and chronic persistent infections are common. Massive use of antibiotics has resulted in β-lactam antibiotic resistance in about 60% infections caused by both nosocomial and community-acquired strains of S. aureus (CA-MRSA). Although most MRSA strains are sensitive to other bactericidal antibiotics, including glycopeptides (GP, e.g., vancomycin) and fluoroquinolones (FQ, e.g., moxifloxacin), some strains show intermediate levels of vancomycin resistance (VISA). S. aureus also evolved resistance mechanisms to powerful daptomycin and linezolid. All clinical trials that sought to address the emergence of drug-resistant S. aureus have thus far failed. Unfortunately, the pace at which antimicrobial therapies are being developed is insufficient to address the problem. S. aureus may soon develop resistance to all available drugs and cause infections without efficient treatment options thus creating an alarming public health situation worldwide.

While the transition of CA-MRSA to the hospital-acquired methicillin-resistant S. aureus (HA-MRSA) is poorly understood, HA-MRSA strains display reduced virulence and lower growth rates that likely represent adaptation to survival during drug exposure. As reduced proliferation of HAMRSA may lower the efficiency of antibiotics that target cell growth and division, the physiological value of active pathways that sustain bacterial viability should increase. Metabolic pathways were successful antimicrobial targets in the past and may continue to be the ones in future. However, recent data suggest the coexistence of metabolic variants among HA-MRSA and targeting metabolically different strains with a single drug could be problematic. An alternative strategy to fight healthcare-associated infections (HAIs) is to target a process that is critical for the mechanism of bacterial resistance to multiple drugs.

H₂S is a toxic gas that has been associated with beneficial functions in mammals, including vasorelaxation, cardioprotection, neurotransmission, and anti-inflammatory action in the gastrointestinal (GI) tract. The ability of H₂S to function as a signaling molecule parallels the action of another established gasotransmitter, nitric oxide (NO). Endogenous NO was demonstrated to protect certain Gram(+) bacteria against antibiotics and oxidative stress.

Like NO, H₂S is produced enzymatically in various tissues, and three H₂S-generating enzymes have been characterized in mammals: cystathionine β-synthase (CBS), cystathionine γ-lyase (CSE) and 3-mercaptopyruvate sulfurtransferase (3MST). CBS and CSE produce H₂S predominantly from L-cysteine (Cys). 3MST does so via the intermediate synthesis of 3-mercaptopyruvate by cysteine aminotranferase (CAT), which is inhibited by aspartate (Asp) competition for Cys on CAT. In contrast to mammals, bacteria-derived H₂S has been known for centuries, but has been considered to be merely a byproduct of sulfur metabolism with no particular physiological function in non-sulfur microorganisms (it is used as energy source in purple and green sulfur bacteria). Further, little is known about the metabolic pathways involving H₂S in mesophilic bacteria. Analysis of bacterial genomes, however, revealed that most, if not all, possess orthologs of mammalian CBS, CSE, or 3MST, suggesting an important cellular function(s) that preserved these genes throughout bacterial evolution.

In addition to inhibition of their direct targets, bactericidal antibiotics also stress bacteria and stimulate the production of highly deleterious hydroxyl radicals, which contributes to bacterial killing by damaging cellular macromolecules. Thus defense mechanisms employed by bacteria to fend off reactive oxygen species (ROS) contribute to resistance to both antibiotic and cell-mediated killing. Inactivation of the bacterial H₂S-producing system results in loss of the protection against oxidative stress and an increased susceptibility of bacteria to various classes of antibiotics.

### SUMMARY OF THE DISCLOSURE

Antibiotics selectively inhibit essential bacterial proteins and molecular machines, and are the most common (or the only efficient) therapeutic option for treating infectious diseases. But, the pace at which antibiotic resistance is emerging may soon result in pathogenic strains resistant to all known antibiotics. Because many antibiotics trigger oxidative damage in bacteria, the inventors developed a strategy to treat bacterial infections by inhibiting H₂S-producing enzymes. Specifically, the inventors found that treating bacterial infections with small molecule inhibitors of H₂S-producing enzymes confers bacterial resistance to oxidative stress and augment the killing effects of conventional antibiotics. Thus, the inventors developed the compounds of the invention that target H₂S-producing system. The H₂S-producing system, unlike other inducible antioxidant systems, does not require de novo protein synthesis. As a result, the compounds of the invention act rapidly and even under conditions of translational shutdown imposed by antibiotics.

Thus, one aspect of the disclosure provides compounds of formula (I):

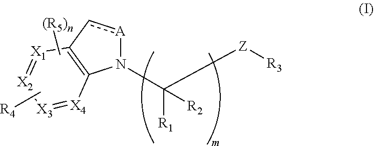

or a pharmaceutically acceptable salt thereof, wherein
[symbol] represents a single or double bond, provided that the bond satisfies the valence requirement of the C atoms;
X₁, X₂, X₃, and X₄ are independently selected from CH and N, provided no more than one of X₁, X₂, X₃, and X₄ is N;
A represents CH, CH₂, N, NH, or C═O;
Z represents —C(O)—, —C(O)NR—,

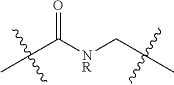

R or a 5-member heteroaryl optionally substituted with one or more R₆,
wherein R is hydrogen or C₁-C₆ alkyl;
m is an integer 1 or 2;
n is an integer 0, 1, 2, 3, or 4;
R, is hydrogen or C₁-C₆ alkyl;
R₂ is hydrogen or C₁-C₆ alkyl; or R, and R₂ groups when attached to the same carbon atom form ═O;
R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), amino(C₁-C₆ alkyl), —CONH—OH, —CONH—OCO(C₁-C₆ alkyl), —CONH—NH₂, —SO₂OH, —SO₂N(R₉)₂, —N(R₉)SO₂R₉, —NHCO—NHSO₂R₉, tertazolyl, oxazolidinedion-5-yl, thiazolidinedion-5-yl, 1,2,4-oxadiazol-5(4H)-one-3-yl, pyrrolidine-2,4-dion-5-yl, and furan-2,4(3H,5H)-dion-5-yl;
R₄ represents hydrogen, halogen, cycloalkyl optionally substituted with one or more R₆, aryl optionally substituted with one or more R₆, heteroaryl optionally substituted with one or more R₆, or heterocyclyl optionally substituted with one or more R₇; and
R₅ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl) optionally substituted with one or more R₈, —NHCO—NH(C₁-C₆ alkyl) optionally substituted with one or more R₈, —N(R₉)SO₂R₉, and —SO₂N(R₉)₂, or two R₅ groups form a heterocyclyl optionally substituted with one or more R₇;
   wherein:
   each R₆ is independently selected from the group consisting of halogen, —NO₂, —CN, C₁-C₆ alkyl optionally substituted with one or more R₈, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), —N(R₉)SO₂R₉, —SO₂N(R₉)₂, aryl(C₀-C₆ alkyl) optionally substituted with one or more R₈, heteroaryl(C₀-C₆ alkyl) optionally substituted with one or more R₈, heterocyclyl(C₀-C₆ alkyl) optionally substituted with one or more R₈, and heterocyclyl(C₁-C₆ alkoxy) optionally substituted with one or more R₈;
   each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), aryl optionally substituted with one or more R₈, heteroaryl optionally substituted with one or more R₈, and heterocyclyl optionally substituted with one or more R₈; or two R₇ groups when attached to the same carbon atom form ═O;
   each R₈ is independently selected from the group consisting of halogen, —NO₂, —CN, C₁-C₆ alkyl, C₁-C₆ haloalkyl, —OH, C₁-C₆ alkoxy, —CO₂H, and C₁-C₆ haloalkoxy; or two R₈ groups when attached to the same carbon atom form ═O; and
   each R₉ is independently selected from the group consisting of hydrogen, C₁-C₆ alkyl, C₁-C₆ haloalkyl, alkoxy(C₁-C₆ alkyl), —COCH₃, cycloalkyl optionally substituted with one or more R₈, aryl optionally substituted with one or more R₈, and heteroaryl optionally substituted with one or more R₈.

Another aspect of the disclosure provides compounds of formula (I) that are represented by formula (II):

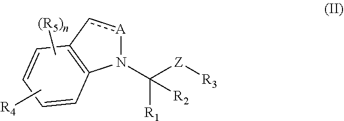

or a pharmaceutically acceptable salt thereof, wherein
[symbol] represents a single or double bond, provided that the bond satisfies the valence requirement of the C atoms;
A represents CH, CH₂, N, NH, or C═O;
Z represents —C(O)—, —C(O)NR—,

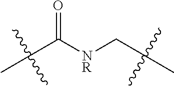

or a 5-member heteroaryl optionally substituted with one or more R₆,
wherein R is hydrogen or C₁-C₆ alkyl;
n is an integer 0, 1, 2, 3, or 4;
R₁ is hydrogen or C₁-C₆ alkyl;
R₂ is hydrogen or C₁-C₆ alkyl; or R, and R₂ groups when attached to the same carbon atom form ═O;
R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), amino(C₁-C₆ alkyl), —CONH—OH, —CONH—OCO(C₁-C₆ alkyl), —CONH—NH₂, —SO₂OH, —SO₂N(R₉)₂, —N(R₉)SO₂R₉, —NHCO—NHSO₂R₉, tertazolyl, oxazolidinedion-5-yl, thiazolidinedion-5-yl, 1,2,4-oxadiazol-5(4H)-one-3-yl, pyrrolidine-2,4-dion-5-yl, and furan-2,4(3H,5H)-dion-5-yl;
R₄ represents hydrogen, halogen, cycloalkyl optionally substituted with one or more R₆, aryl optionally substituted with one or more R₆, heteroaryl optionally substituted with one or more R₆, or heterocyclyl optionally substituted with one or more R₇; and
R₅ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl) optionally substituted with one or more R₈, —NHCO—NH(C₁-C₆ alkyl) optionally substituted with one or more R₈, —N(R₉)SO₂R₉, and —SO₂N(R₉)₂, or two R₅ groups form a heterocyclyl optionally substituted with one or more R₇;
wherein:
   each R₆ is independently selected from the group consisting of halogen, —NO₂, —CN, C₁-C₆ alkyl optionally substituted with one or more R₈, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), —N(R₉)SO₂R₉, —SO₂N(R₉)₂, aryl(C₀-C₆ alkyl) optionally substituted with one or more R₈, heteroaryl(C₀-C₆ alkyl) optionally substituted with one or more R₈, heterocyclyl(C₀-C₆ alkyl) optionally substituted with one or more R₈, and heterocyclyl(C₁-C₆ alkoxy) optionally substituted with one or more R₈;
   each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), aryl optionally substituted with one or more R₈, heteroaryl optionally substituted with one or more R₈, and heterocyclyl optionally substituted with one or more R₈; or two R₇ groups when attached to the same carbon atom form ═O;
   each R₈ is independently selected from the group consisting of halogen, —NO₂, —CN, C₁-C₆ alkyl, C₁-C₆ haloalkyl, —OH, C₁-C₆ alkoxy, —CO₂H, and C₁-C₆ haloalkoxy; or two R₈ groups when attached to the same carbon atom form ═O; and
   each R₉ is independently selected from the group consisting of hydrogen, C₁-C₆ alkyl, C₁-C₆ haloalkyl, alkoxy(C₁-C₆ alkyl), —COCH₃, cycloalkyl optionally substituted with one or more R₈, aryl optionally substituted with one or more R₈, and heteroaryl optionally substituted with one or more R₈.

In certain embodiments of this aspect, the compound of formula (I) or (II) is not:
(2-(6-(6-(pyrrolidin-1-yl)pyridin-3-yl)-1H-indol-1-yl)acetyl)glycine,
(2-(5-(quinolin-3-yl)-1H-indol-1-yl)acetyl)glycine,
(2-(5-(naphthalen-1-yl)-1H-indol-1-yl)acetyl)glycine,
(2-(3-(4-hydroxy-3-isopropylbenzyl)-2-methyl-1H-indol-1-yl)acetyl)glycine,
(2-(4-(5-(3-(trifluoromethyl)-4-((1,1,1-trifluoropropan-2-yl)oxy)phenyl)-1,2,4-oxadiazol-3-yl)-1H-indol-1-yl)acetyl)glycine
(2-(3-(2-aminoethyl)-5-(dibenzo[b,d]furan-4-yl)-1H-indol-1-yl)acetyl)glycine,
(2-(6-chloro-1H-indol-1-yl)acetyl)glycine,
methyl (2-(6-chloro-1H-indol-1-yl)acetyl)glycinate,
(2-(6-bromo-1H-indol-1-yl)acetyl)glycine, or
(2-(5-bromo-1H-indol-1-yl)acetyl)glycine.

Also disclosed herein are pharmaceutical compositions. Examples of such compositions include those having at least one pharmaceutically acceptable carrier, diluent, and/or excipient together with a compound or a pharmaceutically acceptable salt thereof as described herein.

Another aspect of the disclosure provides methods of treating a bacterial infection. Such methods include administering to a subject in need of such treatment one or more disclosed compounds and pharmaceutical compositions.

In certain embodiments of this aspect, the methods also include administering a second antibacterial compound.

Another aspect of the disclosure is the use of the compounds described herein to inhibit one or more H₂S-producing enzymes.

All publications referenced herein are incorporated by reference in their entirety to the extent they are not inconsistent with the teachings presented herein.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1 illustrates the inhibition of S. aureus RN4220 in liquid culture with the compound of Ex. 80 (GO-0001197; labeled as #9077) (30 μM) or with propargylglycine (PAG) (5 mM) in the presence of 10% MIC of gentamicin (Gm).

FIG. 2 illustrates the specificity of a compound of the disclosure for S. aureus H₂S-producing enzymes. (A) Relative inhibition of H₂S synthesis in S. aureus culture in the presence of Ex. 80 (GO-0001197; labeled as #9077). The level of H₂S production without Ex. 80 was set to 100%. (B) Determination of IC₅₀ for Ex. 80 in vitro. (C) Binding curve for Ex. 80 from MicroScale Thermophoresis (MST).

FIG. 3 illustrates inhibition of culture growth of drug-resistant S. aureus by (A) the compound of Ex. 80 (GO-0001197; labeled as #9077) (30 μM) and 50% MIC of methicillin (Met) in methicillin-resistant USA300 strain; or by (B) the compound of Ex. 80 (30 μM) and 50% MIC of vancomycin (Van) in vancomycin-resistant RN10659 strain.

### DETAILED DESCRIPTION

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosed materials and methods provide improvements in treatment of bacterial infections. Specifically, the inventors found that treating bacterial infections with small molecule inhibitors of H₂S-producing enzymes that confer bacterial resistance to oxidative stress and augment the killing effects of conventional antibiotics. Thus, the compounds of the invention target H₂S-producing system, which, unlike other inducible antioxidant systems, do not require de novo protein synthesis, thus acting rapidly and even under conditions of translational shutdown imposed by antibiotics. The compounds of the disclosure are also particularly useful at potentiation of different types of antibiotics, augmentation of low doses of antibiotics, and/or potentiation of antibiotics in strains which developed resistance to these antibiotics, including healthcare-associated pathogens.

Thus, one aspect of the disclosure provides compounds of formula (I):

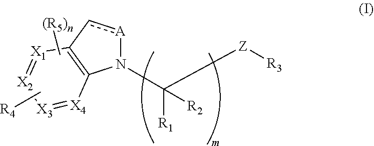

or a pharmaceutically acceptable salt thereof, wherein
[symbol] represents a single or double bond, provided that the bond satisfies the valence requirement of the C atoms;
X₁, X₂, X₃, and X₄ are independently selected from CH and N, provided no more than one of X₁, X₂, X₃, and X₄ is N;
A represents CH, CH₂, N, NH, or C═O;
Z represents —C(O)—, —C(O)NR—,

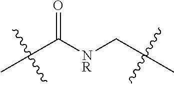

or a 5-member heteroaryl optionally substituted with one or more R₆,
wherein R is hydrogen or C₁-C₆ alkyl;
m is an integer 1 or 2;
n is an integer 0, 1, 2, 3, or 4;
R, is hydrogen or C₁-C₆ alkyl;
R₂ is hydrogen or C₁-C₆ alkyl; or R, and R₂ groups when attached to the same carbon atom form ═O;
R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), amino(C₁-C₆ alkyl), —CONH—OH, —CONH—OCO(C₁-C₆ alkyl), —CONH—NH₂, —SO₂OH, —SO₂N(R₉)₂, —N(R₉)SO₂R₉, —NHCO—NHSO₂R₉, tertazolyl, oxazolidinedion-5-yl, thiazolidinedion-5-yl, 1,2,4-oxadiazol-5(4H)-one-3-yl, pyrrolidine-2,4-dion-5-yl, or furan-2,4(3H,5H)-dion-5-yl;
R₄ represents hydrogen, halogen, cycloalkyl optionally substituted with one or more R₆, aryl optionally substituted with one or more R₆, heteroaryl optionally substituted with one or more R₆, or heterocyclyl optionally substituted with one or more R₇; and
R₅ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl) optionally substituted with one or more R₈, —NHCO—NH(C₁-C₆ alkyl) optionally substituted with one or more R₈, —N(R₉)SO₂R₉, and —SO₂N(R₉)₂, or two R₅ groups form a heterocyclyl optionally substituted with one or more R₇;
   wherein:
   each R₆ is independently selected from the group consisting of halogen, —NO₂, —CN, C₁-C₆ alkyl optionally substituted with one or more R₈, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), —N(R₉)SO₂R₉, —SO₂N(R₉)₂, aryl(C₀-C₆ alkyl) optionally substituted with one or more R₈, heteroaryl(C₀-C₆ alkyl) optionally substituted with one or more R₈, heterocyclyl(C₀-C₆ alkyl) optionally substituted with one or more R₈, and heterocyclyl(C₁-C₆ alkoxy) optionally substituted with one or more R₈;
   each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), aryl optionally substituted with one or more R₈, heteroaryl optionally substituted with one or more R₈, and heterocyclyl optionally substituted with one or more R₈; or two R₇ groups when attached to the same carbon atom form ═O;
   each R₈ is independently selected from the group consisting of halogen, —NO₂, —CN, C₁-C₆ alkyl, C₁-C₆ haloalkyl, —OH, C₁-C₆ alkoxy, —CO₂H, and C₁-C₆ haloalkoxy; or two R₈ groups when attached to the same carbon atom form ═O; and
   each R₉ is independently selected from the group consisting of hydrogen, C₁-C₆ alkyl, C₁-C₆ haloalkyl, alkoxy(C₁-C₆ alkyl), —COCH₃, cycloalkyl optionally substituted with one or more R₈, aryl optionally substituted with one or more R₈, and heteroaryl optionally substituted with one or more R₈.

In some embodiments, the compounds of formula (I) as otherwise described herein are those wherein m is 1.

In some embodiments, the compounds of formula (I) as otherwise described herein are those wherein m is 2.

In some embodiments, the compounds of formula (I) as otherwise described herein are those wherein X₁, X₂, X₃, and X₄ are independently CH.

In some embodiments, the compounds of formula (I) as otherwise described herein are those wherein one of X₁, X₂, X₃, and X₄ is N, and the remaining substituents are independently CH.

Another aspect of the disclosure provides compounds of formula (I) as otherwise described herein that are represented by formula (II):

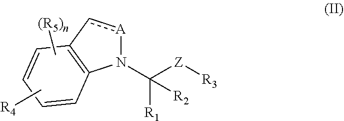

or a pharmaceutically acceptable salt thereof, wherein
[symbol] represents a single or double bond, provided that the bond satisfies the valence requirement of the C atoms;
A represents CH, CH₂, N, NH, or C═O;
Z represents —C(O)—, —C(O)NR—,

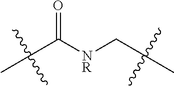

or a 5-member heteroaryl optionally substituted with one or more R₆,
wherein R is hydrogen or C₁-C₆ alkyl;
n is an integer 0, 1, 2, 3, or 4;
R, is hydrogen or C₁-C₆ alkyl;
R₂ is hydrogen or C₁-C₆ alkyl; or R₁ and R₂ groups when attached to the same carbon atom form ═O;
R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), amino(C₁-C₆ alkyl), —CONH—OH, —CONH—OCO(C₁-C₆ alkyl), —CONH—NH₂, —SO₂OH, —SO₂N(R₉)₂, —N(R₉)SO₂R₉, —NHCO—NHSO₂R₉, tertazolyl, oxazolidinedion-5-yl, thiazolidinedion-5-yl, 1,2,4-oxadiazol-5(4H)-one-3-yl, pyrrolidine-2,4-dion-5-yl, or furan-2,4(3H,5H)-dion-5-yl;
R₄ represents hydrogen, halogen, cycloalkyl optionally substituted with one or more R₆, aryl optionally substituted with one or more R₆, heteroaryl optionally substituted with one or more R₆, or heterocyclyl optionally substituted with one or more R₇; and
R₅ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl) optionally substituted with one or more R₈, —NHCO—NH(C₁-C₆ alkyl) optionally substituted with one or more R₈, —N(R₉)SO₂R₉, and —SO₂N(R₉)₂, or two R₅ groups form a heterocyclyl optionally substituted with one or more R₇;
   wherein:
   each R₆ is independently selected from the group consisting of halogen, —NO₂, —CN, C₁-C₆ alkyl optionally substituted with one or more R₈, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), —N(R₉)SO₂R₉, —SO₂N(R₉)₂, aryl(C₀-C₆ alkyl) optionally substituted with one or more R₈, heteroaryl(C₀-C₆ alkyl) optionally substituted with one or more R₈, heterocyclyl(C₀-C₆ alkyl) optionally substituted with one or more R₈, and heterocyclyl(C₁-C₆ alkoxy) optionally substituted with one or more R₈;
   each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), aryl optionally substituted with one or more R₈, heteroaryl optionally substituted with one or more R₈, and heterocyclyl optionally substituted with one or more R₈; or two R₇ groups when attached to the same carbon atom form ═O;
   each R₈ is independently selected from the group consisting of halogen, —NO₂, —CN, C₁-C₆ alkyl, C₁-C₆ haloalkyl, —OH, C₁-C₆ alkoxy, —CO₂H, and C₁-C₆ haloalkoxy; or two R₈ groups when attached to the same carbon atom form ═O; and
   each R₉ is independently selected from the group consisting of hydrogen, C₁-C₆ alkyl, C₁-C₆ haloalkyl, alkoxy(C₁-C₆ alkyl), —COCH₃, cycloalkyl optionally substituted with one or more R₈, aryl optionally substituted with one or more R₈, and heteroaryl optionally substituted with one or more R₈.

In certain embodiments of this aspect, the compound of formula (I) or (II) as otherwise described herein is not:
(2-(6-(6-(pyrrolidin-1-yl)pyridin-3-yl)-1H-indol-1-yl)acetyl)glycine,
(2-(5-(quinolin-3-yl)-1H-indol-1-yl)acetyl)glycine,
(2-(5-(naphthalen-1-yl)-1H-indol-1-yl)acetyl)glycine,
(2-(3-(4-hydroxy-3-isopropylbenzyl)-2-methyl-1H-indol-1-yl)acetyl)glycine,
(2-(4-(5-(3-(trifluoromethyl)-4-((1,1,1-trifluoropropan-2-yl)oxy)phenyl)-1,2,4-oxadiazol-3-yl)-1H-indol-1-yl)acetyl)glycine
(2-(3-(2-aminoethyl)-5-(dibenzo[b,d]furan-4-yl)-1H-indol-1-yl)acetyl)glycine,
(2-(6-chloro-1H-indol-1-yl)acetyl)glycine,
methyl (2-(6-chloro-1H-indol-1-yl)acetyl)glycinate,
(2-(6-bromo-1H-indol-1-yl)acetyl)glycine, or
(2-(5-bromo-1H-indol-1-yl)acetyl)glycine.

In certain embodiments, the compounds of formula (II) as otherwise described herein are those where [symbol] represents a double bond, e.g., the compound of formula (III):

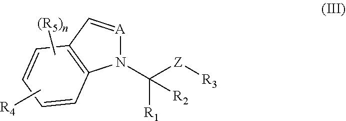

In one embodiment, the disclosure provides compounds of formula (II) as otherwise described herein wherein A is CH. Such compounds are of formula (IV):

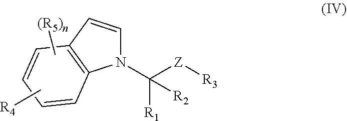

In one embodiment, the disclosure provides compounds of formula (III) as otherwise described herein wherein A is N. Such compounds are of formula (V):

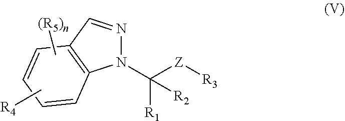

In certain embodiments, the compounds of formula (I) as otherwise described herein are those where [symbol] represents a single bond, e.g., the compound of formula (VI):

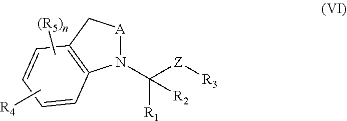

In one embodiment, the disclosure provides compounds of formula (VI) as otherwise described herein wherein A is C═O. Such compounds are of formula (VII):

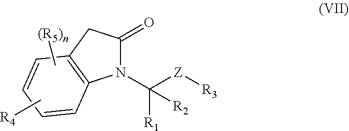

Another embodiment of the invention provides compounds of formula (I)-(VII) as otherwise described herein wherein Z is —C(O)— or —C(O)NR—. In certain embodiments, Z is —C(O)—. In certain embodiments, Z is —C(O)NR—.

Another embodiment of the invention provides compounds of formula (I)-(VII) as otherwise described herein wherein Z is

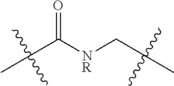

or a 5-member heteroaryl optionally substituted with one or more R₆.

Another embodiment of the invention provides compounds of formula (I)-(VII) as otherwise described herein wherein Z is

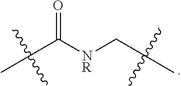

For example, in certain embodiments, R is hydrogen. In other embodiments, R is methyl.

Another embodiment of the invention provides compounds of formula (I)-(VII) as otherwise described herein wherein Z is a 5-member heteroaryl optionally substituted with one or more R₆. For example, in certain embodiments, Z is:

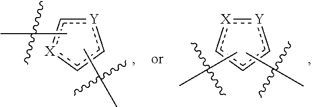

or wherein
X represents O, S, or N;
Y represents CR, N, or NR, wherein
[symbol] represents a single or double bond, provided that the bonds satisfy the valence requirements of the C, N and O atoms; and
each R independently is hydrogen or C₁-C₆ alkyl.

In certain embodiments of the disclosure, the compound of formula (I)-(VII) as otherwise described herein is wherein Z is:

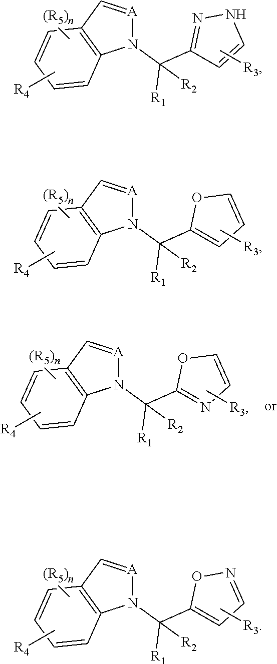

For example, in certain embodiments, Z is:

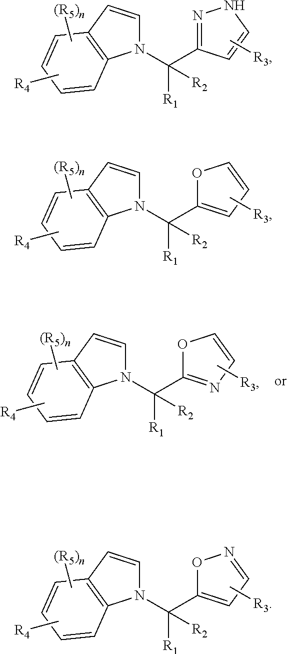

In certain embodiments of the disclosure, the compound of formula (I)-(VII) as otherwise described herein is wherein Z is:

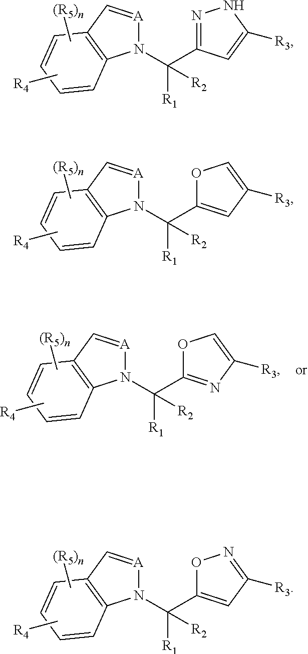

For example, in certain embodiments, Z is:

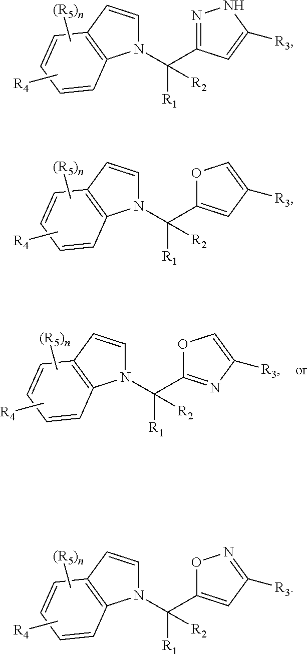

Another embodiment of the invention provides compounds of formula (I)-(VII) as otherwise described herein wherein R₁ is hydrogen. In certain embodiments R₁ is methyl.

Another embodiment of the invention provides compounds of formula (I)-(VII) as otherwise described herein wherein R₂ is hydrogen. In certain embodiments R₂ is methyl.

For example, in certain embodiment of the disclosure, both R, and R₂ are hydrogen in the compounds of formula (I)-(VII) as otherwise described herein.

Another embodiment of the invention provides compounds of formula (I)-(VII) as otherwise described herein wherein R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), amino(C₁-C₆ alkyl), —CONH—OH, —CONH—OCO(C₁-C₆ alkyl), —CONH—NH₂, —N(R₉)SO₂R₉, and —NHCO—NHSO₂R₉. Other embodiments are those where R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), and amino(C₁-C₆ alkyl). Other embodiments are those where R₃ is selected from the group consisting of C₁-C₆ alkyl, —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), and —CON(C₁-C₆ alkyl)₂. In still other embodiments, R₃ is selected from the group consisting of C₁-C₆ alkyl, —CO₂H, —CO₂(C₁-C₆ alkyl), —CO₂(arylC₁-C₆ alkyl), —CO₂(aryl), —CHO, hydroxy(C₁-C₆ alkyl), alkoxy(C₁-C₆ alkyl), and amino(C₁-C₆ alkyl). In still other embodiments, R₃ is selected from the group consisting of C₁-C₆ alkyl, —CO₂H, —CO₂(C₁-C₆ alkyl), —CO₂(arylC₁-C₆ alkyl), —CO₂(aryl), and —CHO. In still other embodiments, R₃ is selected from the group consisting of —CO₂H, —CO₂(C₁-C₆ alkyl), —CO₂(arylC₁-C₆ alkyl), —CO₂(aryl), and —CHO. Other embodiments are those where R₃ is selected from the group consisting of —CO₂H, —CO₂(C₁-C₆ alkyl), and —CHO. For example, in certain embodiment of the disclosure, R₃ is —CO₂H or —CO₂(C₁-C₆ alkyl). In certain embodiment of the disclosure R₃ is —CO₂H.

Another embodiment of the invention provides compounds of formula (I)-(VII) as otherwise described herein where n is an integer 0, 1, or 2. For example, in certain embodiment of the disclosure, n is 0 or 1. In certain embodiment of the disclosure, n is 0. In certain embodiment of the disclosure, n is 1.

Another embodiment of the invention provides compounds of formula (I)-(VII) as otherwise described herein wherein R₅ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy. For example, in certain embodiment of the disclosure, R₅ is independently selected from the group consisting of halogen, C₁-C₆ alkyl, C₁-C₆ haloalkyl, —OH, C₁-C₆ alkoxy, and C₁-C₆ haloalkoxy. In certain embodiment of the disclosure, R₅ is independently selected from the group consisting of halogen, C₁-C₆ alkyl, and C₁-C₆ haloalkyl. For example, in certain embodiments, R₅ is halogen. For example, R₅ is Cl or Br; or R₅ is Cl; or R₅ is Br.

Another embodiment of the invention provides compounds of formula (I)-(VII) as otherwise described herein wherein R₄ represents hydrogen. For example, in some embodiments, such compounds may be of formula:

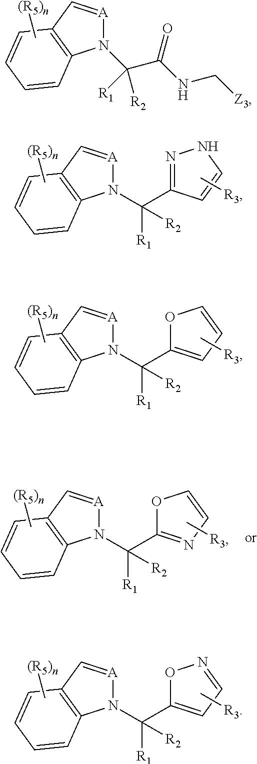

For example, in some embodiments, such compounds may be of formula:

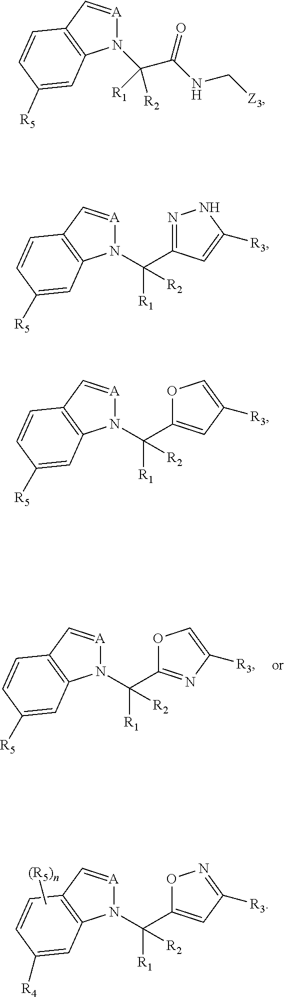

Another embodiment of the invention provides compounds of formula (I)-(VII) as otherwise described herein wherein R₄ represents hydrogen, aryl optionally substituted with one or more R₆, or heteroaryl optionally substituted with one or more R₆. For example, in certain embodiment of the disclosure, R₄ represents aryl optionally substituted with one or more R₆ or heteroaryl optionally substituted with one or more R₆. In certain embodiment of the disclosure, R₄ represents aryl optionally substituted with one or more R₆ or heteroaryl optionally substituted with one or more R₆. In certain embodiment of the disclosure, R₄ represents aryl optionally substituted with one or more R₆. Other embodiments are those where R₄ represents phenyl optionally substituted with one or more R₆. In certain embodiment of the disclosure, R₄ represents heteroaryl optionally substituted with one or more R₆. In certain embodiment of the disclosure, R₄ represents benzothiophene optionally substituted with one or more R₆.

Some particular embodiments include those wherein each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl). Other embodiments are those where each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy. In certain embodiments, each R₆ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy. In certain embodiments, each R₆ is independently selected from the group consisting of halogen, C₁-C₆ alkyl, and C₁-C₆ haloalkyl. For example, in certain embodiments, R₆ is halogen. For example, R₆ is Cl or Br; or R₆ is Cl; or R₆ is Br.

### Therapeutics Applications

The disclosure also provides methods of bacterial infections. Such method includes administering to a subject in need of such treatment an effective amount of one or more compounds of the disclosure (i.e., compounds of formula (I)-(VII)).

In certain embodiment, the method also includes administering a second antibacterial compound. Such second antibacterial compounds may be, but are not limited to, a quinolone, an acridine, a phenothiazine, an aminoglycoside, a macrolide, an amphenicol, a steroid, an ansamycin, an antifolate, a polymyxin, a glycopeptide, a cephalosporin, a lactam, and any combination thereof. In certain embodiments, the second antibacterial compound may be administered in an amount below its minimum inhibitory concentration (MIC) established in the absence of the one or more compounds. “Minimum inhibitory concentration” or “MIC” as used herein, means is the lowest concentration of an agent (e.g., a chemical compound) that prevents visible microorganism growth (e.g., bacterium growth) after an overnight incubation. For example, the second antibacterial compound may be administered in an amount less than 1% of, e.g., less than 10%, or less than 25%, or less than 50%, or less than 75%, or even less than 90% of its minimum inhibitory concentration (MIC).

Some particular bacteria of the disclosure includes, but is not limited to, bacterial genera selected from Bacillus, Brucella, Clostridium, Enterococcus, Escherichia, Francisella, Helicobacter, Klebsiella, Legionella, Listeria, Mycobacterium, Pseudomonas, Salmonella, Shigella, Staphylococcus, Streptococcus, Trypanasoma, Vibrio, and Yersinia.

The bacterial infections that may be treated by the compositions of the disclosure include, but are not limited to, pneumonia, bronchitis, diphtheria, pertussis (whooping cough), tetanus, endocarditis, sepsis, bacterial gastroenteritis, cholera, tuberculosis, gonorrhea, chlamydia, syphilis, bacterial meningitis, trachoma, lyme disease, and leprosy.

### Pharmaceutical Compositions

In another aspect, the present disclosure provides compositions comprising one or more of compounds as described above with respect to formula (I)-(VII) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds. In certain embodiments, the composition may include one or more antibiotic compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001, 0.001 or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Definitions

The following terms and expressions used herein have the indicated meanings.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Terms used herein may be preceded and/or followed by a single dash, “−”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)(OC₁-C₆alkyl) indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “alkylene” refers to a bivalent alkyl group. In some embodiments, an “alkylene” is a polymethylene group, i.e., —(CH₂)ₙ—, wherein n is a positive integer, preferably from one to six, from one to four, from one to three, from one to two, or from two to three. A substituted alkylene is a bivalent alkyl group in which one or more hydrogen atoms is replaced with a substituent. Suitable substituents include those described below for a substituted aliphatic group. An alkylene also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means —Cl, —Br, —I or —F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The terms “heterocyclyl” and “heterocycloalkyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thia” as used herein means a ═S group.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art.

“Treating” or “treatment” as used herein covers the treatment of a disease or disorder described herein, in a subject, preferably a human, and includes:

i. inhibiting a disease or disorder, i.e., arresting its development;

ii. relieving a disease or disorder, i.e., causing regression of the disorder;

iii. slowing progression of the disorder; and/or

iv. inhibiting, relieving, ameliorating, or slowing progression of one or more symptoms of the disease or disorder

“Subject” refers to a warm blooded animal such as a mammal, preferably a human, or a human child, which is afflicted with, or has the potential to be afflicted with one or more diseases and disorders described herein.

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/I, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art and as described herein. For example, compounds of structural formula (I) can be prepared according to Procedures A-C (below), or analogous synthetic procedures. One of skill in the art can adapt the reaction sequences of Procedures A-C and Examples 1-80 to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

Procedure A: To a cooled −10° C. solution of compound 7 (see scheme in Example 4) (0.523 g, 2 mmol) in pyridine (2 mL) the corresponding sulfonyl chloride (2.4 mmol) was added and the mixture was stirred overnight at room temperature. After the reaction completed, pyridine was evaporated under reduced pressure. 5% aqueous HCl (3 mL) was added to the residue and the mixture was stirred for 1 h under ice-bath cooling. The precipitate was filtered off and the filtrate was extracted with DCM. The organic layers were evaporated to yield slightly impure compounds, which were used in the next steps without further purification.

Procedure B: To a solution/suspension of methyl ester (˜2 mmol) in MeOH (2 mL) 10% aqueous LiOH (1.2 mL) was added and the mixture was stirred overnight at room temperature. Then reaction mixture was diluted with water (7 mL) and acidified with 10% aqueous HCl. The precipitated solid was filtered, washed with water, and purified by column chromatography to obtain the target compound.

Procedure C: A mixture of 6-bromo-1H-indole (0.500 g, 2.55 mmol), and t-BuOK (0.343 g, 3.06 mmol) in DMSO (1.5 mL) was stirred at room temperature for 30 min and then treated with a suitable alkylating agent (2.80 mmol). In certain embodiments, suitable alkylating agents include, but are not limited to:

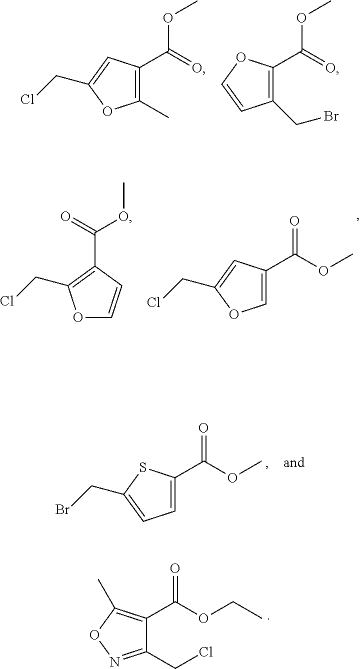

The resulting mixture was stirred until the NMR of an aliquot revealed completion of the reaction (approx. 2-4 h) and then diluted with 30% aqueous KOH. The reaction mass was left to stir overnight at room temperature, diluted with water (15 mL), and acidified to pH 2-3 with 10% hydrochloric acid. The precipitate was filtered, washed with water, 2-propanol, and hexane, dried under vacuum and purified by silica gel column chromatography (if necessary).

### EXAMPLES

The preparation of the compounds of the disclosure is illustrated further by the following examples, which are not to be construed as limiting the disclosure in scope or spirit to the specific procedures and compounds described in them. In all cases, unless otherwise specified, the column chromatography is performed using a silica gel solid phase.

### Example 1: 2-{2-[6-(7-chloro-1-benzothiophen-2-yl)-1H-indol-1-yl]acetamido}acetic acid (GO-0001194)

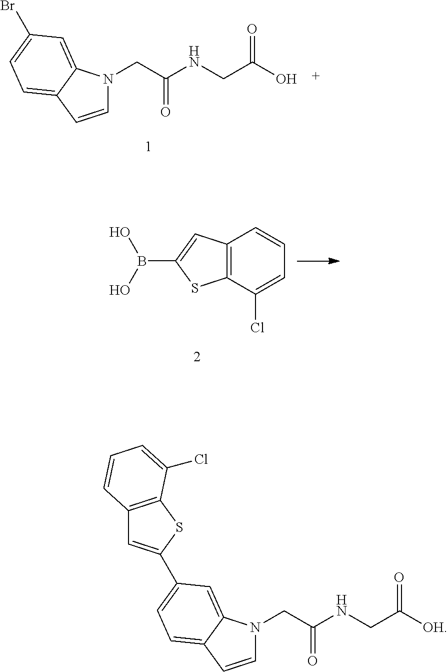 [SMILES: O=C(O)CNC(=O)Cn1ccc2ccc(Br)cc21 ; OB(O)c1cc2cccc(Cl)c2s1 ; O=C(O)CNC(=O)Cn1ccc2ccc(-c3cc4cccc(Cl)c4s3)cc21]

To a mixture of compound 1 (0.780 g, 2.50 mmol), compound 2 (0.700 g, 3.30 mmol), Na₂CO₃ (0.800 g, 7.50 mmol), dioxane (20 mL), and water (5 mL) Pd(dppf)Cl₂ (0.100 g) was added and the reaction mass was heated under reflux for 4 h. The volatiles were evaporated and the residue was dissolved in water (20 mL). The solution was extracted with dichloromethane (2×20 mL). The aqueous layer was acidified to pH 2-3 and the precipitated solid was collected by filtration. The filter cake was washed with water, cold 2-propanol and hexane, and dried under vacuum to obtain 0.570 g (1.43 mmol, 57%) of target compound. ¹H NMR (400 MHz, DMSO-d₆) δ 8.47 (s, 1H), 7.92 (m, 3H), 7.81 (d, J=4.9 Hz, 1H), 7.65 (d, J=8.2 Hz, 1H), 7.50 (d, J=8.4 Hz, 1H), 7.44 (m, 3H), 6.51 (s, 1H), 5.01 (s, 2H), 3.79 (d, J=5.6 Hz, 2H), COOH is not observed.

### Example 2: 5-[(6-bromo-1H-indol-1-yl)methyl]-1,2-oxazole-3-carboxylic acid (GO-0001195)

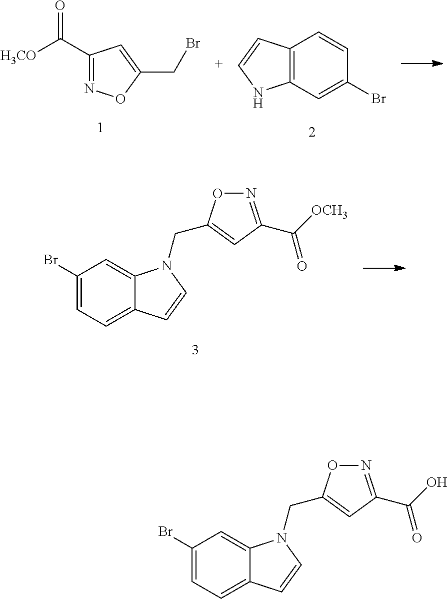 [SMILES: COC(=O)c1cc(CBr)on1 ; Brc1ccc2cc[nH]c2c1 ; COC(=O)c1cc(Cn2ccc3ccc(Br)cc32)on1 ; O=C(O)c1cc(Cn2ccc3ccc(Br)cc32)on1]

### Step 2.1: Preparation of methyl 5-[(6-bromo-1H-indol-1-yl)methyl]-1,2-oxazole-3-carboxylate

A solution of compound 2 (0.305 g, 1.56 mmol) and t-BuOK (0.210 g, 1.87 mmol) in DMSO (1 mL) was stirred for 30 min at room temperature before compound 1 (0.376 g, 1.71 mmol) was added thereto. The reaction was stirred for 16 h at room temperature and then poured into water (20 mL). The resulting mixture was extracted with DCM (3×30 mL). The combined organic layers were washed with water, dried over Na₂SO₄, and evaporated under reduced pressure to obtain compound 3, which was used as is in the next step.

### Step 2.2: Preparation of 5-[(6-bromo-1H-indol-1-yl)methyl]-1,2-oxazole-3-carboxylic acid

To a solution of compound 3, obtained in the previous step, in methanol (20 mL) 20% aqueous KOH (1 mL) was added and the reaction was stirred at room temperature for 2 h. The volatiles were evaporated and the residue was dissolved in water (10 mL). The pH of the solution was adjusted to 2-3 with 10% aqueous hydrochloric acid. The product was extracted with DCM (2×30 mL). The combined organics were washed with water (2×20 mL), dried over Na₂SO₄, and evaporated under reduced pressure. The crude residue was purified by column chromatography to obtain 0.155 g (0.482 mmol, 31% over 2 steps) of target compound. ¹H NMR (400 MHz, DMSO-d₆) δ 14.11 (s, 1H), 7.90 (s, 1H), 7.53 (m, 2H), 7.19 (d, J=8.4 Hz, 1H), 6.68 (s, 1H), 6.55 (d, J=3.3 Hz, 1H), 5.73 (s, 2H).

### Example 3: 2-[(6-bromo-1H-indol-1-yl)methyl]-1,3-oxazole-4-carboxylic acid (GO-0001198)

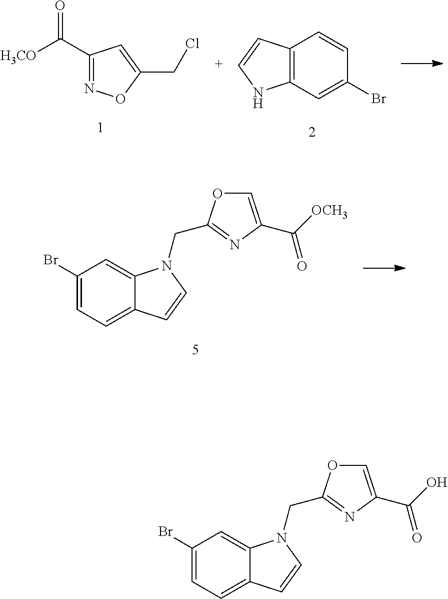 [SMILES: COC(=O)c1cc(CCl)on1 ; Brc1ccc2cc[nH]c2c1 ; COC(=O)c1coc(Cn2ccc3ccc(Br)cc32)n1 ; O=C(O)c1coc(Cn2ccc3ccc(Br)cc32)n1]

### Step 3.1: Preparation of methyl 2-[(6-bromo-1H-indol-1-yl)methyl]-1,3-oxazole-5-carboxylate

A solution of compound 2 (0.305 g, 1.56 mmol) and t-BuOK (0.210 g, 1.87 mmol) in DMSO (1 mL) was stirred for 30 min at room temperature before compound 4 (0.300 g, 1.71 mmol) was added thereto. The reaction was stirred for 18 h at room temperature and then poured into water (20 mL). The resulting mixture was extracted with DCM (3×30 mL). The combined organic layers were washed with water, dried over Na₂SO₄, and evaporated under reduced pressure to obtain compound 5, which was used as is in the next step.

### Step 3.2: Preparation of 2-[(6-bromo-1H-indol-1-yl)methyl]-1,3-oxazole-4-carboxylic acid

To a solution of compound 5, obtained in the previous step, in methanol (20 mL) 20% aqueous KOH (1 mL) was added and the reaction was stirred at room temperature for 2 h. The volatiles were evaporated and the residue was dissolved in water (10 mL). The pH of the solution was adjusted to 2-3 with 10% aqueous hydrochloric acid. The product was extracted with DCM (2×30 mL). The combined organics were washed with water (2×20 mL), dried over Na₂SO₄, and evaporated under reduced pressure. The crude residue was purified by column chromatography to obtain 0.029 g (0.090 mmol, 6% over 2 steps) of target compound. ¹H NMR (400 MHz, Chloroform-d) δ 8.24 (s, 1H), 7.62 (s, 1H), 7.48 (d, J=8.4 Hz, 1H), 7.23 (m, 1H), 7.19 (d, J=3.2 Hz, 1H), 6.54 (d, J=3.2 Hz, 1H), 5.42 (s, 2H), COOH is not observed.

### Example 4:5-[(6-bromo-1H-indol-1-yl)methyl]-1H-pyrazole-3-carboxylic acid (GO-0001199)

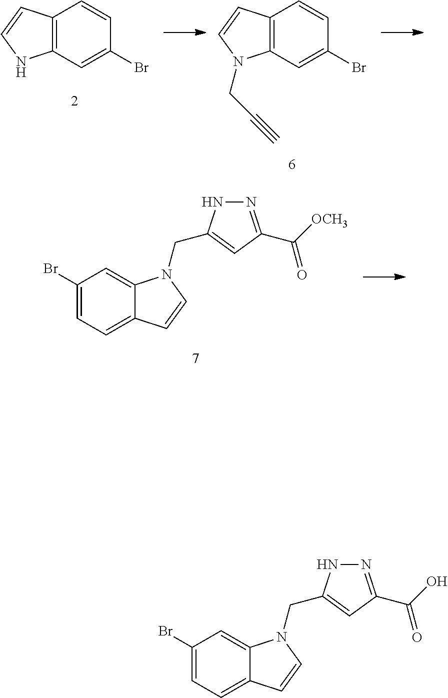 [SMILES: Brc1ccc2cc[nH]c2c1 ; C#CCn1ccc2ccc(Br)cc21 ; COC(=O)c1cc(Cn2ccc3ccc(Br)cc32)[nH]n1 ; O=C(O)c1cc(Cn2ccc3ccc(Br)cc32)[nH]n1]

### Step 4.1: Preparation of 6-bromo-1-(prop-2-yn-1-yl)-1H-indole

To a solution of 5-bromoindole (1.96 g, 10.0 mmol) and propargyl bromide (2.23 g of 80% solution in toluene; 15.0 mmol) in toluene (30 mL) tetrabutylammonium bromide (0.161 g, 0.500 mmol) and 50% aqueous NaOH (6 mL) were added. The two-phase system was stirred vigorously for 3 h at room temperature and then diluted with toluene (10 mL). The organic layers were separated, washed with water (2×20 mL) and brine (2×30 mL), dried over Na₂SO₄, and evaporated under reduced pressure to give 2.00 g (85.4 mmol, 85%) of compound 6 as an oil.

### Step 4.2: Preparation of ethyl 5-[(6-bromo-1H-indol-1-yl)methyl]-1H-pyrazole-3-carboxylate

To a solution of compound 6 (0.500 g, 2.14 mmol) in toluene (10 mL) ethyl diazoacetate (0.244 g, 2.14 mmol) was added. The mixture was heated at reflux for 12 h and concentrated in vacuo. The residue was purified by column chromatography to give 0.100 g (0.287 mmol, 14%) of compound 7.

### Step 4.3: Preparation of 5-[(6-bromo-1H-indol-1-yl)methyl]-1H-pyrazole-3-carboxylic acid

A solution of compound 7 (0.100 g, 0.287 mmol) in methanol was treated with a solution of NaOH (0.040 g, 1.00 mmol) in water, and heated to reflux. After 10 h the solution was concentrated, diluted with water, and acidified with 1N aqueous hydrochloric acid. The precipitate was filtered and dried in air to afford 0.040 g (0.125 mmol, 43%) of target compound as a brown solid. ¹H NMR (400 MHz, DMSO-d₆) δ 13.71 (br. s, 2H), 7.80 (s, 1H), 7.48 (m, 2H), 7.13 (dd, J=8.5, 1.7 Hz, 1H), 6.56 (s, 1H), 6.47 (d, J=3.2 Hz, 1H), 5.39 (s, 2H).

### Example 5: 2-[2-(6-bromo-1H-indazol-1-yl)acetamido]acetic acid (GO-0001200)

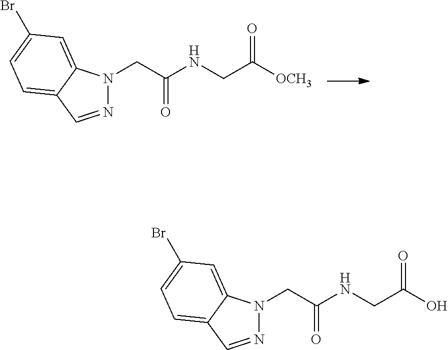 [SMILES: COC(=O)CNC(=O)Cn1ncc2ccc(Br)cc21 ; O=C(O)CNC(=O)Cn1ncc2ccc(Br)cc21]

A mixture of compound Example 6 (0.420 g, 1.29 mmol), LiOH.H₂O (0.060 g, 1.40 mmol), THF (2 mL), and water (0.5 mL) was stirred for 2 h at room temperature, then diluted with water, and acidified to pH 2-3 with 10% hydrochloric acid. The precipitated solid was filtered, washed with water and dried in vacuo to obtain 0.330 g (1.06 mmol, 83%) of target compound. ¹H NMR (400 MHz, DMSO-d₆) δ 12.64 (s, 1H), 8.51 (t, J=5.8 Hz, 1H), 8.09 (s, 1H), 7.94 (s, 1H), 7.72 (d, J=8.5 Hz, 1H), 7.26 (d, J=8.4 Hz, 1H), 5.17 (s, 2H), 3.80 (d, J=5.8 Hz, 2H).

### Example 6: Methyl 2-[2-(6-bromo-1H-indazol-1-yl)acetamido]acetate (GO-0001201)

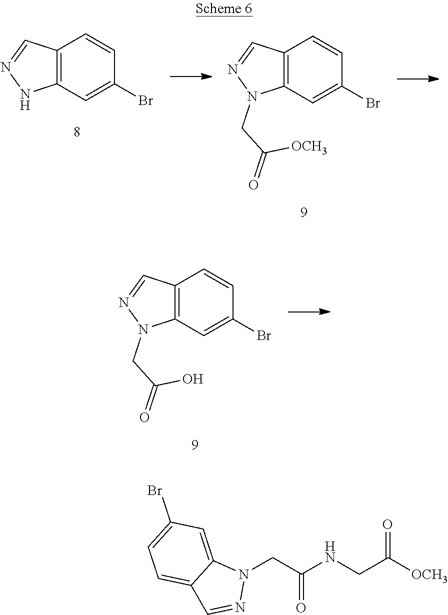

### Step 6.1 Preparation of methyl 2-(6-bromo-1H-indazol-1-yl)acetate

A suspension of compound 8 (1.47 g, 7.40 mmol) and Cs₂CO₃ (4.85 g, 14.9 mmol) in dry DMF (10 mL) was stirred for 30 min and then treated with methyl bromoacetate (1.48 g, 9.70 mmol). The resulting mixture was stirred at 50° C. overnight and then evaporated under reduced pressure. The residue was partitioned between water (30 mL) and dichloromethane (50 mL). The water layer was separated and extracted with dichloromethane (2×50 mL). The combined organic layers were washed with water, dried over Na₂SO₄, and evaporated under reduced pressure.

### Step 6.2 Preparation of 2-(6-bromo-1H-indazol-1-yl)acetic acid

The residue of 9 was dissolved in water (20 mL) and 20% aqueous KOH (5 mL) was added thereto. The resulting mixture was stirred at room temperature for 2 h and evaporated under reduced pressure. The solid thus obtained was dissolved in water and the solution was acidified until pH 2-3 with 10% hydrochloric acid. The precipitated solid was filtered, washed with water (2×20 mL) and cold isopropanol, and dried to obtain 0.550 g (2.16 mmol, 29%) of compound 10.

### Step 6.3 Preparation of methyl 2-[2-(6-bromo-1H-indazol-1-yl)acetamido]acetate

To a cooled to 0° C. mixture of compound 10 (0.540 g, 2.12 mmol), glycine methyl ester hydrochloride (0.292 g, 2.30 mmol), triethylamine (0.260 g, 2.57 mmol), and a solution of HOBt in DMF (10% wt., 3 mL) EDC (0.378 g, 2.43 mmol) was added and the reaction was stirred overnight at room temperature. Then water was added until a suspension formed. The resulting mixture was extracted with dichloromethane (2×30 mL). The combined organic layers were washed with water, dried over Na₂SO₄, and evaporated under reduced pressure to obtain 0.470 g (1.44 mmol, 68%) of target compound. ¹H NMR (500 MHz, DMSO-d₆) δ 8.63 (s, 1H), 8.11 (s, 1H), 7.94 (s, 1H), 7.73 (d, J=8.4 Hz, 1H), 7.27 (dd, J=8.4, 1.6 Hz, 1H), 5.19 (s, 2H), 3.90 (d, J=5.8 Hz, 2H), 3.63 (s, 3H).

### Examples 7-42: Preparation of Indol Derivatives

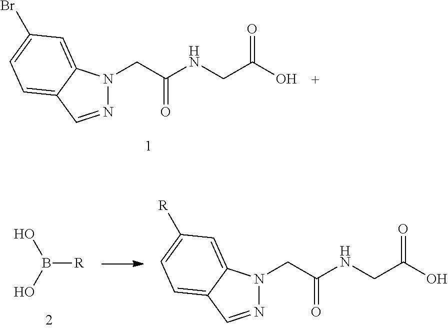

Synthesis was carried out on a 150 mg scale: a small vial was charged with 1 eq of arylbromide, 1-1.2 eq of boronic acid derivative, 3 mol % Pd(dppf)Cl₂ and 2 eq of Na₂CO₃. The reaction mixture was diluted with 2 mL of dioxane and 0.5 mL of water and heated at 90-95° C. for 16-18 hours. Reaction progress was monitored by LC-MS. After completion the solvent was removed under reduced pressure and the solid residue was mixed with water and sonicated. The resulting precipitate was filtered, washed with water (2×5 mL) and dried. If the precipitate did not form, the solution was extracted with CH₂Cl₂. The CH₂Cl₂ solution was washed with water (2×4 mL), dried over Na₂SO₄ and evaporated to dryness. The crude product was subjected purification on HPLC (H₂O-MeOH).

Purification was performed using HPLC (H₂O-MeOH; Agilent 1260 Infinity systems equipped with DAD and mass-detectors. Waters Sunfire C18 OBD Prep Column, 100 Å, 5 μm, 19 mm×100 mm with SunFire C18 Prep Guard Cartridge, 100 Å, 10 μm, 19 mm×10 mm) The material was dissolved in 0.7 mL DMSO. Flow: 30 mL/min. Purity of the obtained fractions was determined via the analytical LCMS. Spectra were recorded for each fraction as it was obtained immediately after chromatography in the solution form. The solvent was evaporated in the flow of N₂ at 80° C. On the basis of post-chromatography LCMS analysis, fractions were united. Solid fractions were dissolved in 0.5 mL MeOH and transferred into pre-weighed, marked vials. The solutions obtained were again evaporated in the flow of N₂ at 80° C. After drying, products were characterized by LCMS.

| | | | | LCMS |
|---|---|---|---|---|
| | | | | (retention |
| Ex. | Molecule | | | time and |
| No. | Name | Chemical structure | Chemical name | peaks) |
| 7 | GO-0001202 | 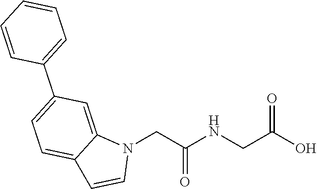 [SMILES: O=C(O)CNC(=O)Cn1ccc2ccc(-c3ccccc3)cc21] | (2-(6-phenyl-1H-indol-1- yl)acetyl)glycine | 1.304 min; 309.2 [M + H]⁺, 307.2 [M − H]⁻ |
| 8 | GO-0001203 | 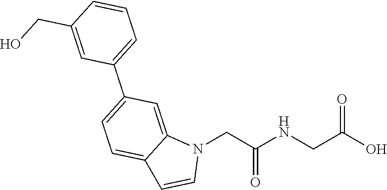 [SMILES: O=C(O)CNC(=O)Cn1ccc2ccc(-c3cccc(CO)c3)cc21] | (2-(6-(3- (hydroxymethyl)phenyl)- 1H-indol-1- yl)acetyl)glycine | 1.151 min; 339.2 [M + H]⁺, 337.2 (NEG) |
| 9 | GO-0001204 | 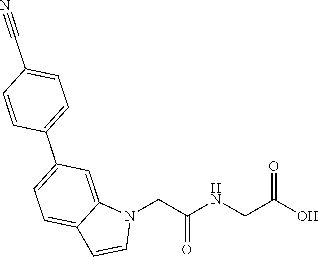 [SMILES: N#Cc1ccc(-c2ccc3ccn(CC(=O)NCC(=O)O)c3c2)cc1] | (2-(6-(4-cyanophenyl)- 1H-indol-1- yl)acetyl)glycine | 1.234 min; 334.2 [M + H]⁺, 332.2 [M − H]⁻ |
| 10 | GO-0001205 | 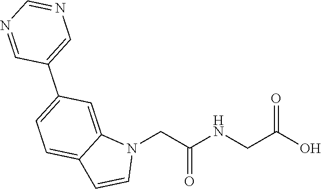 [SMILES: O=C(O)CNC(=O)Cn1ccc2ccc(-c3cncnc3)cc21] | (2-(6-(pyrimidin-5-yl)- 1H-indol-1- yl)acetyl)glycine | 1.195 min; 311.2 [M + H]⁺, 309.2 [M − H]⁻ |
| 11 | GO-0001206 | 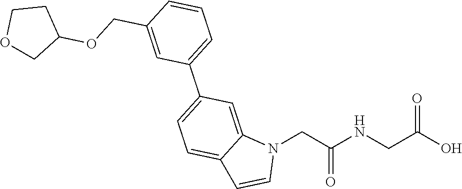 [SMILES: O=C(O)CNC(=O)Cn1ccc2ccc(-c3cccc(COC4CCOC4)c3)cc21] | (2-(6-(3- (((tetrahydrofuran-3- yl)oxy)methyl)phenyl)- 1H-indol-1- yl)acetyl)glycine | 1.263 min; 409.2 [M + H]⁺, 407.2 [M − H]⁻ |
| 12 | GO-0001207 | 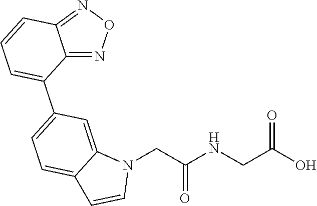 [SMILES: O=C(O)CNC(=O)Cn1ccc2ccc(-c3cccc4nonc34)cc21] | (2-(6- (benzo[c][1,2,5]oxadiazol- 4-yl)-1H-indol-1- yl)acetyl)glycine | 1.299 min; 351.2 [M + H]⁺, 349.2 [M − H]⁻ |
| 13 | GO-0001208 | 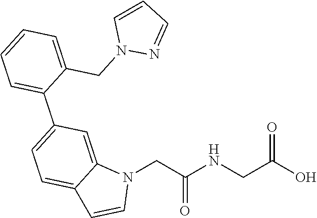 [SMILES: O=C(O)CNC(=O)Cn1ccc2ccc(-c3ccccc3Cn3cccn3)cc21] | (2-(6-(2-((1H-pyrazol-1- yl)methyl)phenyl)-1H- indol-1-yl)acetyl)glycine | 1.282 min; 389.2 [M + H]⁺, 387.2 [M − H]⁻ |
| 14 | GO-0001209 | 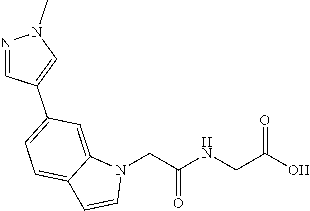 [SMILES: Cn1cc(-c2ccc3ccn(CC(=O)NCC(=O)O)c3c2)cn1] | (2-(6-(1-methyl-1H- pyrazol-4-yl)-1H-indol-1- yl)acetyl)glycine | 1.018 min; 313.2 [M + H]⁺, 311.2 [M − H]⁻ |
| 15 | GO-0001210 | 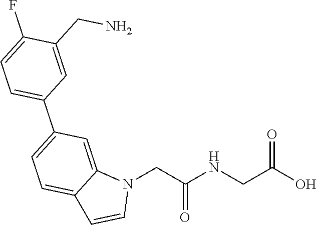 [SMILES: NCc1cc(-c2ccc3ccn(CC(=O)NCC(=O)O)c3c2)ccc1F] | (2-(6-(3-(aminomethyl)- 4-fluorophenyl)-1H- indol-1-yl)acetyl)glycine | 0.954 min; 356.2 [M + H]⁺, 354.2 [M − H]⁻ |
| 16 | GO-0001211 | 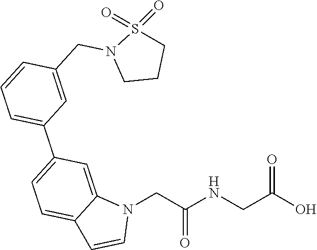 [SMILES: O=C(O)CNC(=O)Cn1ccc2ccc(-c3cccc(CN4CCCS4(=O)=O)c3)cc21] | (2-(6-(3-((1,1- dioxidoisothiazolidin-2- yl)methyl)phenyl)-1H- indol-1-yl)acetyl)glycine | 1.202 min; 442.2 [M + H]⁺, 440.2 [M − H]⁻ |
| 17 | GO-0001212 | 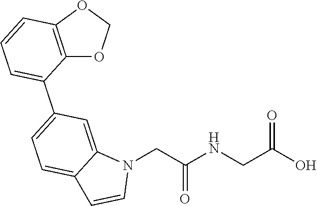 [SMILES: O=C(O)CNC(=O)Cn1ccc2ccc(-c3cccc4c3OCO4)cc21] | (2-(6- (benzo[d][1,3]dioxol-4- yl)-1H-indol-1- yl)acetyl)glycine | 1.302 min; 353.2 [M + H]⁺, 351.2 [M − H]⁻ |
| 18 | GO-0001213 | 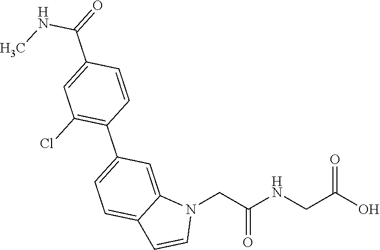 [SMILES: CNC(=O)c1ccc(-c2ccc3ccn(CC(=O)NCC(=O)O)c3c2)c(Cl)c1] | (2-(6-(2-chloro-4- (methylcarbamoyl) phenyl)-1H-indol-1- yl)acetyl)glycine | 1.144 min; 400.2 [M + H]⁺, 399.2 [M − H]⁻ |
| 19 | GO-0001214 | 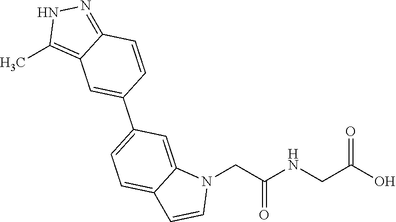 [SMILES: Cc1[nH]nc2ccc(-c3ccc4ccn(CC(=O)NCC(=O)O)c4c3)cc12] | (2-(6-(3-methyl-2H- indazol-5-yl)-1H-indol-1- yl)acetyl)glycine | 1.188 min; 363.2 [M + H]⁺, 311.2 [M − H]⁻ |
| 20 | GO-0001215 | 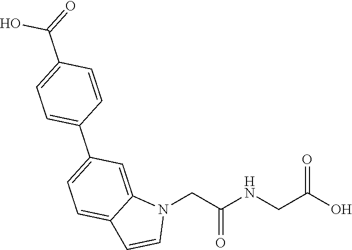 [SMILES: O=C(O)CNC(=O)Cn1ccc2ccc(-c3ccc(C(=O)O)cc3)cc21] | 4-(1-(2- ((carboxymethyl)amino)- 2-oxoethyl)-1H-indol-6- yl)benzoic acid | 1.131 min; 353.2 [M + H]⁺, 351.2 [M − H]⁻ |
| 21 | GO-0001216 | 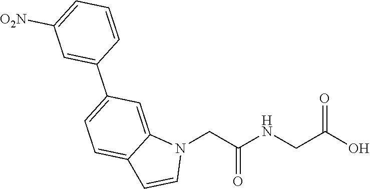 [SMILES: O=C(O)CNC(=O)Cn1ccc2ccc(-c3cccc([N+](=O)[O-])c3)cc21] | (2-(6-(3-nitrophenyl)-1H- indol-1-yl)acetyl)glycine | 1.104 min; 354.2 [M + H]⁺, 352.2 [M − H]⁻ |
| 22 | GO-0001217 | 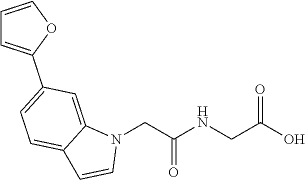 [SMILES: O=C(O)CNC(=O)Cn1ccc2ccc(-c3ccco3)cc21] | (2-(6-(furan-2-yl)-1H- indol-1-yl)acetyl)glycine | 1.230 min; 299.2 [M + H]⁺, 298.2 [M − H]⁻ |
| 23 | GO-0001218 | 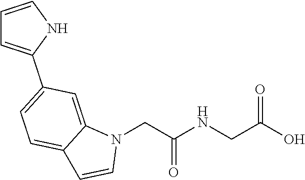 [SMILES: O=C(O)CNC(=O)Cn1ccc2ccc(-c3ccc[nH]3)cc21] | (2-(6-(1H-pyrrol-2-yl)- 1H-indol-1- yl)acetyl)glycine | 1.144 min; 298.2 [M + H]⁺, 296.2 [M − H]⁻ |
| 24 | GO-0001219 | 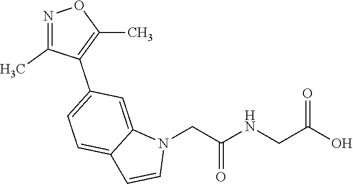 [SMILES: Cc1noc(C)c1-c1ccc2ccn(CC(=O)NCC(=O)O)c2c1] | (2-(6-(3,5- dimethylisoxazol-4-yl)- 1H-indol-1- yl)acetyl)glycine | 1.163 min; 328.2 [M + H]⁺, 326.2 [M − H]⁻ |
| 25 | GO-0001220 | 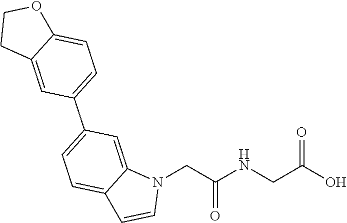 [SMILES: O=C(O)CNC(=O)Cn1ccc2ccc(-c3ccc4c(c3)CCO4)cc21] | (2-(6-(2,3- dihydrobenzofuran-5-yl)- 1H-indol-1- yl)acetyl)glycine | 1.087 min; 351.2 [M + H]⁺, 349.2 [M − H]⁻ |
| 26 | GO-0001221 | 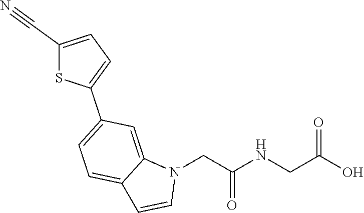 [SMILES: N#Cc1ccc(-c2ccc3ccn(CC(=O)NCC(=O)O)c3c2)s1] | (2-(6-(5-cyanothiophen- 2-yl)-1H-indol-1- yl)acetyl)glycine | 1.252 min; 340.2 [M + H]⁺, 338.2 [M − H]⁻ |
| 27 | GO-0001222 | 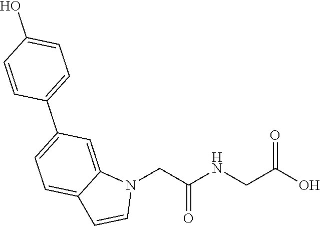 [SMILES: O=C(O)CNC(=O)Cn1ccc2ccc(-c3ccc(O)cc3)cc21] | (2-(6-(4-hydroxyphenyl)- 1H-indol-1- yl)acetyl)glycine | 1.111 min; 325.2 [M + H]⁺, 323.2 [M − H]⁻ |
| 28 | GO-0001223 | 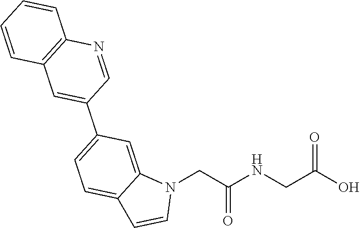 [SMILES: O=C(O)CNC(=O)Cn1ccc2ccc(-c3cnc4ccccc4c3)cc21] | (2-(6-(quinolin-3-yl)-1H- indol-1-yl)acetyl)glycine | 1.070 min; 360.2 [M + H]⁺, 358.2 [M − H]⁻ |
| 29 | GO-0001224 | 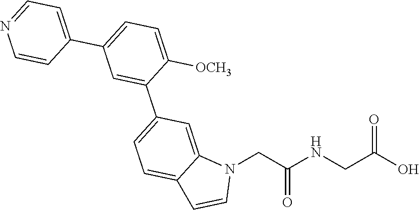 [SMILES: COc1ccc(-c2ccncc2)cc1-c1ccc2ccn(CC(=O)NCC(=O)O)c2c1] | (2-(6-(2-methoxy-5- (pyridin-4-yl)phenyl)-1H- indol-1-yl)acetyl)glycine | 1.029 min; 416.4 [M + H]⁺, 413.8 [M − H]⁻ |
| 30 | GO-0001225 | 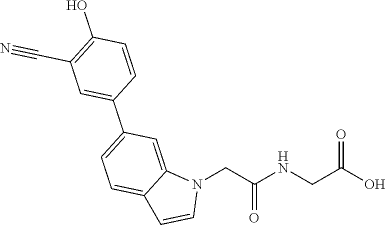 [SMILES: N#Cc1cc(-c2ccc3ccn(CC(=O)NCC(=O)O)c3c2)ccc1O] | (2-(6-(3-cyano-4- hydroxyphenyl)-1H- indol-1-yl)acetyl)glycine | 1.121 min; 350.2 [M + H]⁺, 349.2 [M − H]⁻ |
| 31 | GO-0001226 | 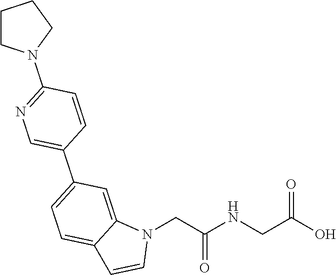 [SMILES: O=C(O)CNC(=O)Cn1ccc2ccc(-c3ccc(N4CCCC4)nc3)cc21] | (2-(6-(6-(pyrrolidin-1- yl)pyridin-3-yl)-1H-indol- 1-yl)acetyl)glycine | 0.960 min; 379.2 [M + H]⁺, 377.2 [M − H]⁻ |
| 32 | GO-0001227 | 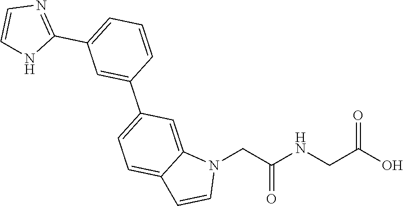 [SMILES: O=C(O)CNC(=O)Cn1ccc2ccc(-c3cccc(-c4ncc[nH]4)c3)cc21] | (2-(6-(3-(1H-imidazol-2- yl)phenyl)-1H-indol-1- yl)acetyl)glycine | 0.966 min; 375.2 [M + H]⁺, 381.8 [M − H]⁻ |
| 33 | GO-0001228 | 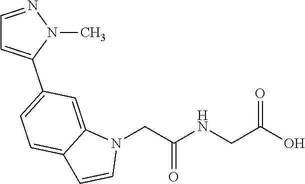 [SMILES: Cn1nccc1-c1ccc2ccn(CC(=O)NCC(=O)O)c2c1] | (2-(6-(1-methyl-1H- pyrazol-5-yl)-1H-indol-1- yl)acetyl)glycine | 1.052 min; 313.0 [M + H]⁺, 311.0 [M − H]⁻ |
| 34 | GO-0001229 | 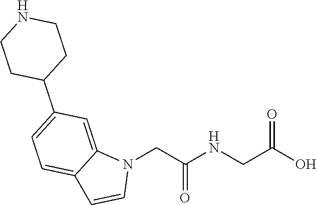 [SMILES: O=C(O)CNC(=O)Cn1ccc2ccc(C3CCNCC3)cc21] | (2-(6-(piperidin-4-yl)-1H- indol-1-yl)acetyl)glycine | 0.713 min; 316.2 [M + H]⁺, 314.2 [M − H]⁻ |
| 35 | GO-0001230 | 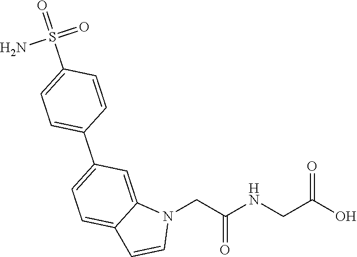 [SMILES: NS(=O)(=O)c1ccc(-c2ccc3ccn(CC(=O)NCC(=O)O)c3c2)cc1] | (2-(6-(4- sulfamoylphenyl)-1H- indol-1-yl)acetyl)glycine | 1.060 min; 388.0 [M + H]⁺, 386.0 [M − H]⁻ |
| 36 | GO-0001231 | 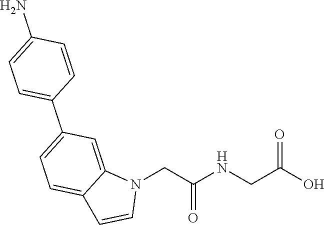 [SMILES: Nc1ccc(-c2ccc3ccn(CC(=O)NCC(=O)O)c3c2)cc1] | (2-(6-(4-aminophenyl)- 1H-indol-1- yl)acetyl)glycine | 0.924 min; 324.2 [M + H]⁺, 322.2 [M − H]⁻ |
| 37 | GO-0001232 | 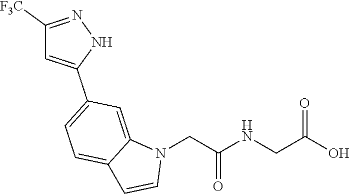 [SMILES: O=C(O)CNC(=O)Cn1ccc2ccc(-c3cc(C(F)(F)F)n[nH]3)cc21] | (2-(6-(3- (trifluoromethyl)-1H- pyrazol-5-yl)-1H-indol-1- yl)acetyl)glycine | 1.230 min; 367.0 [M + H]⁺, 365.0 [M − H]⁻ |
| 38 | GO-0001233 | 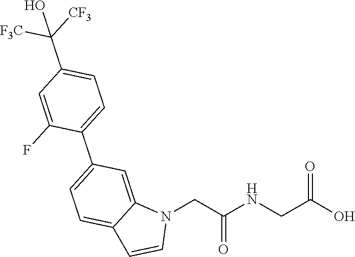 [SMILES: O=C(O)CNC(=O)Cn1ccc2ccc(-c3ccc(C(O)(C(F)(F)F)C(F)(F)F)cc3F)cc21] | (2-(6-(2-fluoro-4- (1,1,1,3,3,3-hexafluoro- 2-hydroxypropan-2- yl)phenyl)-1H-indol-1- yl)acetyl)glycine | 1.175 min; 385.0 [M + H]⁺, 383.1 [M − H]⁻ |
| 39 | GO-0001234 | 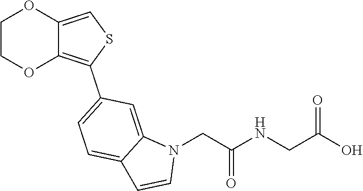 [SMILES: O=C(O)CNC(=O)Cn1ccc2ccc(-c3scc4c3OCCO4)cc21] | (2-(6-(2,3- dihydrothieno[3,4- b][1,4]dioxin-5-yl)-1H- indol-1-yl)acetyl)glycine | 1.160 min; 373.0 [M + H]⁺, 371.0 [M − H]⁻ |
| 40 | GO-0001235 | 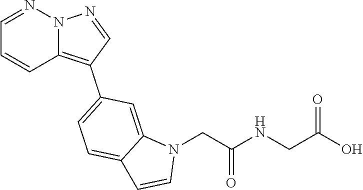 [SMILES: O=C(O)CNC(=O)Cn1ccc2ccc(-c3cnn4ncccc34)cc21] | (2-(6-(pyrazolo[1,5- b]pyridazin-3-yl)-1H- indol-1-yl)acetyl)glycine | 1.071 min; 350.2 [M + H]⁺, 348.2 [M − H]⁻ |
| 41 | GO-0001236 | 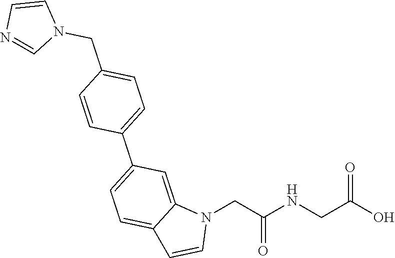 [SMILES: O=C(O)CNC(=O)Cn1ccc2ccc(-c3ccc(Cn4ccnc4)cc3)cc21] | (2-(6-(4-((1H-imidazol-1- yl)methyl)phenyl)-1H- indol-1-yl)acetyl)glycine | 0.864 min; 389.1 [M + H]⁺ |
| 42 | GO-0001237 | 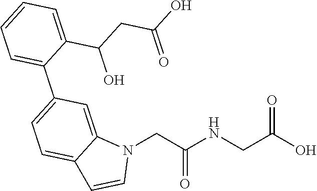 [SMILES: O=C(O)CNC(=O)Cn1ccc2ccc(-c3ccccc3C(O)CC(=O)O)cc21] | 3-(2-(1-(2- ((carboxymethyl)amino)- 2-oxoethyl)-1H-indol-6- yl)phenyl)-3- hydroxypropanoic acid | 1.144 min; 400.2 [M + H]⁺, 399.2 [M − H]⁻ |

### Example 43: 2-(2-{2H,5H-[1,3]dioxolo[4,5-f]indol-5-yl}acetamido)propanoic acid (GO-0001244)

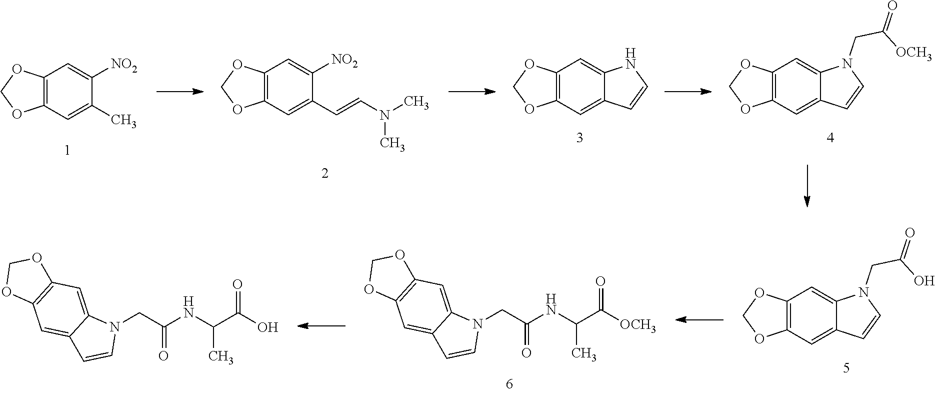

### Step 43.1: Preparation of dimethyl[(E)-2-(6-nitro-2H-1,3-benzodioxol-5-yl)ethenyl]amine

To a solution of compound 1 (5.75 g, 31.7 mmol) in DMF (30 mL) N,N-dimethylformamide dimethyl acetal (5.70 g, 47.6 mmol) was added and the reaction was stirred for 18 hours at 165° C. under argon atmosphere. The residue was recrystallized from ethanol to obtain 3.70 g (15.7 mmol, 49%) of compound 2.

### Step 43.2: Preparation of 2H,5H-[1,3]dioxolo[4,5-f]indole

A mixture of compound 2 (3.70 g, 15.7 mmol), Pd/C (1.00 g, 10 wt. %), and THF (100 mL) was stirred under H₂ atmosphere for 3 hours and then filtered through a small pad of silica gel. The filtrate was evaporated and the residue was used in the next step without further purification.

### Step 43.3: Preparation of methyl 2-{2H,5H-[1,3]dioxolo[4,5-f]indol-5-yl}acetate

To a solution of compound 3, obtained in the previous step, in THF (50 mL) t-BuOK (2.10 g, 18.6 mmol) was added and the mixture was stirred for 30 min at room temperature. Then the reaction was cooled to 0° C. and a solution of ethyl bromoacetate (2.60 g, 17.1 mmol) in THF (15 mL) was added thereto. The resulting mixture was stirred for 4 hours at room temperature and evaporated under reduced pressure.

### Step 43.4: Preparation of 2-{2H,5H-[1,3]dioxolo[4,5-f]indol-5-yl}acetic acid

The residue of 4 was dissolved in MeOH (20 mL) and added to 30% aqueous KOH (10 mL). The mixture was stirred overnight at room temperature and evaporated under reduced pressure. The residue was dissolved in water. The solution was washed with EtOAc (2×50 mL) and acidified to pH 2-3 with 10% aqueous HCl. The resulting suspension was extracted with DCM (3×50 mL). The combined organic layers were dried over Na₂SO₄ and evaporated under reduced pressure to give 0.800 g (3.65 mmol, 24% over three steps) of compound 5.

### Step 43.5: Preparation of methyl 2-(2-{2H,5H-[1,3]dioxolo[4,5-f]indol-5-yl}acetamido)propanoate

To a cooled to 0° C. mixture of compound 5 (0.360 g, 1.60 mmol), alanine methyl ester hydrochloride (0.252 g, 1.80 mmol), HOBt (0.243 g, 1.80 mmol), and triethylamine (0.183 g, 1.80 mmol) in DMF (2 mL) EDC (0.281 g, 1.80 mmol) was added and the mixture was stirred for 3 hours at room temperature. Then it was diluted with water (5 mL) and extracted with DCM (3×10 mL). The combined organic layers were dried over Na₂SO₄ and evaporated under reduced pressure.

### Step 43.6: Preparation of 2-(2-{2H,5H-[1,3]dioxolo[4,5-f]indol-5-yl}acetamido)propanoic acid

The residue of 6 was dissolved in THF (3 mL) and mixed with LiOH.H₂O (0.084 g, 2.00 mmol). The mixture was stirred for 16 hours at room temperature and evaporated under reduced pressure. The residue was dissolved in water (5 mL) and acidified to pH 2-3 with aqueous hydrochloric acid. The precipitated solid was filtered, washed with water (2×5 mL) and dried to obtain 0.175 g (0.603 mmol, 37% over 2 steps) of target compound. ¹H NMR (400 MHz, DMSO-d₆) δ 8.50 (d, J=7.3 Hz, 1H), 7.11 (d, J=3.1 Hz, 1H), 6.98 (s, 1H), 6.93 (s, 1H), 6.27 (d, J=3.2 Hz, 1H), 5.92 (s, 2H), 4.73 (AB-system, 2H), 4.21 (p, J=7.4 Hz, 1H), 1.30 (d, J=7.2 Hz, 3H), COOH is not observed.

### Examples 44-56

The following compounds were prepared substantially according to the procedures described above:

| | | | | LCMS |
|---|---|---|---|---|
| | | | | (retention |
| Ex. | Molecule | | | time and |
| No. | Name | Chemical structure | Chemical name | peaks) |
| 44 | GO-0003545 | 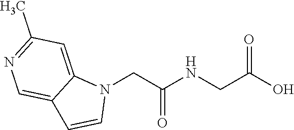 [SMILES: Cc1cc2c(ccn2CC(=O)NCC(=O)O)cn1] | (2-(6-methyl-1H- pyrrolo[3,2-c]pyridin-1- yl)acetyl)glycine | 1.39 min; 248.09 [M + H]⁺ |
| 45 | GO-0003546 | 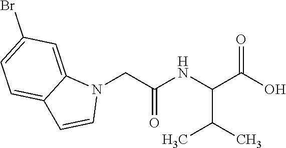 [SMILES: CC(C)C(NC(=O)Cn1ccc2ccc(Br)cc21)C(=O)O] | (2-(6-bromo-1H-indol-1- yl)acetyl)valine | |
| 46 | GO-0003547 | 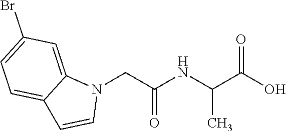 [SMILES: CC(NC(=O)Cn1ccc2ccc(Br)cc21)C(=O)O] | (2-(6-bromo-1H-indol-1- yl)acetyl)alanine | |
| 47 | GO-0003548 | 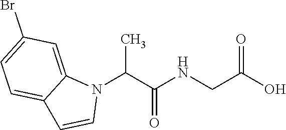 [SMILES: CC(C(=O)NCC(=O)O)n1ccc2ccc(Br)cc21] | (2-(6-bromo-1H-indol-1- yl)propanoyl)glycine | |
| 48 | GO-0003549 | 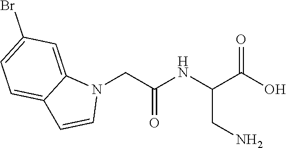 [SMILES: NCC(NC(=O)Cn1ccc2ccc(Br)cc21)C(=O)O] | 3-amino-2-(2-(6-bromo- 1H-indol-1- yl)acetamido)propanoic acid | 1.44 min; 339.84 [M]⁺ |
| 49 | GO-0003550 | 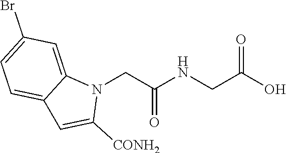 [SMILES: NC(=O)c1cc2ccc(Br)cc2n1CC(=O)NCC(=O)O] | (2-(6-bromo-2-carbamoyl- 1H-indol-1- yl)acetyl)glycine | 1.56 min; 353.75 [M]⁺ |
| 50 | GO-0003551 | 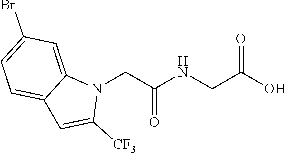 [SMILES: O=C(O)CNC(=O)Cn1c(C(F)(F)F)cc2ccc(Br)cc21] | (2-(6-bromo-2- (trifluoromethyl)-1H-indol- 1-yl)acetyl)glycine | 1.52 min; 379.00 [M]⁺ |
| 51 | GO-0003552 | 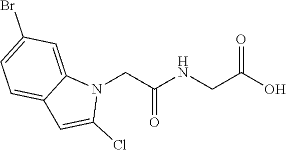 [SMILES: O=C(O)CNC(=O)Cn1c(Cl)cc2ccc(Br)cc21] | (2-(6-bromo-2-chloro-1H- indol-1-yl)acetyl)glycine | |
| 52 | GO-0003553 | 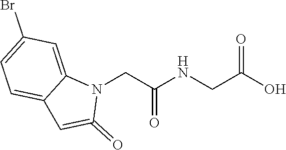 | (2-(6-bromo-2-oxoindolin- 1-yl)acetyl)glycine | |
| 53 | GO-0003554 | 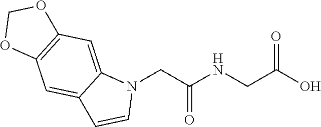 [SMILES: O=C(O)CNC(=O)Cn1ccc2cc3c(cc21)OCO3] | (2-(5H-[1,3]dioxolo[4,5- f]indol-5-yl)acetyl)glycine | 1.48 min; 277.05 [M + H]⁺ |
| 54 | GO-0003556 | 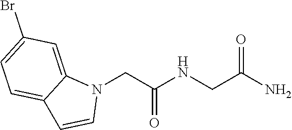 [SMILES: NC(=O)CNC(=O)Cn1ccc2ccc(Br)cc21] | N-(2-amino-2-oxoethyl)-2- (6-bromo-1H-indol-1- yl)acetamide | |
| 55 | GO-0003557 | 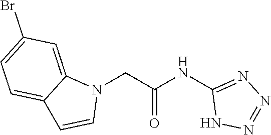 [SMILES: O=C(Cn1ccc2ccc(Br)cc21)Nc1nnn[nH]1] | 2-(6-bromo-1H-indol-1-yl)- N-(1H-tetrazol-5- yl)acetamide | |
| 56 | GO-0003558 | 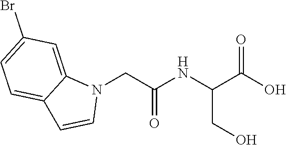 [SMILES: O=C(Cn1ccc2ccc(Br)cc21)NC(CO)C(=O)O] | (2-(6-bromo-1H-indol-1- yl)acetyl)serine | |

### Example 57: 2-[2-(5-bromo-1H-indol-3-yl)acetamido]acetic acid (GO-0003559)

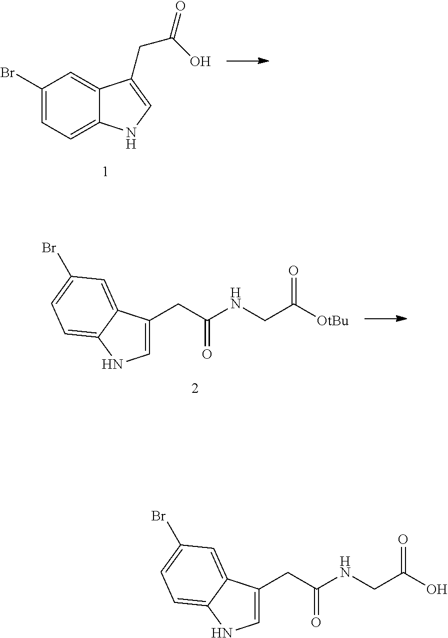 [SMILES: O=C(O)Cc1c[nH]c2ccc(Br)cc12 ; CC(C)(C)OC(=O)CNC(=O)Cc1c[nH]c2ccc(Br)cc12 ; O=C(O)CNC(=O)Cc1c[nH]c2ccc(Br)cc12]

### Step 57.1: Preparation of tert-butyl 2-[2-(5-bromo-1H-indol-3-yl)acetamido]acetate

A mixture of compound 1 (7.15 g, 28.1 mmol), and CDI (4.56 g, 28.1 mmol) in dry MeCN (50 mL) was stirred for 30 min at room temperature. To the resulting solution triethylamine (3.42 g, 33.7 mmol) and glycine tert-butyl ester hydrochloride (4.95 g, 29.5 mmol) were added and the reaction was stirred for 16 hours at 50° C. The solvent was evaporated and the residue was partitioned between water (100 mL) and DCM (150 mL). The layers were separated. The organic phase was washed with water (2×100 mL), dried over Na₂SO₄, and evaporated under reduced pressure to yield crude compound 2, which was used in the next step without further purification.

### Step 57.2 Preparation of 2-[2-(5-bromo-1H-indol-3-yl)acetamido]acetic acid

A mixture of compound 2, obtained in the previous step, TFA (20 mL), and DCM (80 mL) was vigorously stirred for 2 hours. The volatiles were evaporated and the residue was purified by means of column chromatography to obtain 2.10 g (6.75 mmol, 24% over 2 steps) of target compound. ¹H NMR (400 MHz, DMSO-d₆) δ 12.36 (br. s, 1H), 10.97 (s, 1H), 8.07 (t, J=5.8 Hz, 1H), 7.71 (d, J=1.9 Hz, 1H), 7.26 (d, J=8.6 Hz, 1H), 7.21 (d, J=2.4 Hz, 1H), 7.11 (dd, J=8.6, 1.9 Hz, 1H), 3.73 (d, J=5.8 Hz, 2H), 3.52 (s, 2H).

### Example 58:2-{2-[6-(3-methoxypropanamido)-1H-indol-1-yl]acetamido}acetic acid (GO-0003560)

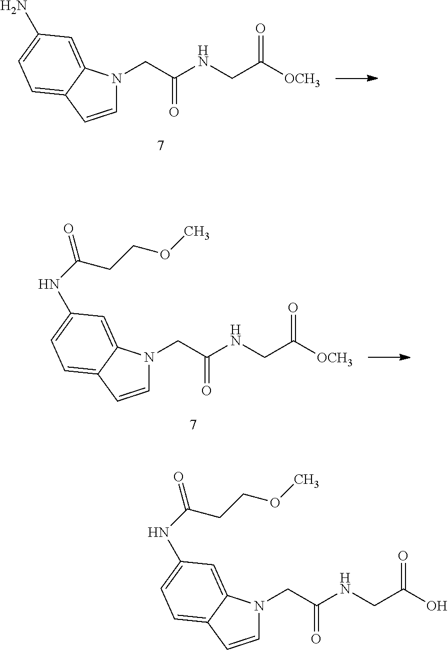 [SMILES: COC(=O)CNC(=O)Cn1ccc2ccc(N)cc21 ; COCCC(=O)Nc1ccc2ccn(CC(=O)NCC(=O)OC)c2c1 ; COCCC(=O)Nc1ccc2ccn(CC(=O)NCC(=O)O)c2c1]

### Step 58.1: Preparation of methyl 2-{2-[6-(3-methoxypropanamido)-1H-indol-1-yl]acetamido}acetate

To a cooled to 0° C. mixture of compound 7 (1.00 g, 3.83 mmol), 3-methoxy-propionic acid (0.478 g, 4.59 mmol), HOBt (600 mg), and DMF (3 mL) EDC (0.743 g, 4.79 mmol) was added and the mixture was stirred overnight at room temperature. Water was then added and the resulting precipitate was filtered to obtain 1.12 g (3.22 mmol, 84%) of compound 8.

### Step 58.2: Preparation of 2-{2-[6-(3-methoxypropanamido)-1H-indol-1-yl]acetamido}acetic acid

2-{2-[6-(3-Methoxypropanamido)-1H-indol-1-yl]acetamido}acetic acid was obtained from compound 8 according to Procedure B. ¹H NMR (400 MHz, DMSO-d₆) δ 12.63 (s, 1H), 9.87 (s, 1H), 8.41 (m, 1H), 7.79 (s, 1H), 7.43 (d, J=7.4 Hz, 1H), 7.25 (t, J=2.7 Hz, 1H), 7.10 (d, J=8.5 Hz, 1H), 6.37 (d, J=3.4 Hz, 1H), 4.81 (s, 2H), 3.80 (d, J=5.5 Hz, 2H), 3.62 (t, J=6.1 Hz, 2H), 3.32 (s, 2H), 3.25 (d, J=2.2 Hz, 3H).

### Example 59: 2-[2-(6-{[(2-methoxyethyl)carbamoyl]amino}-1H-indol-1-yl)acetamido]acetic acid (GO-0003561)

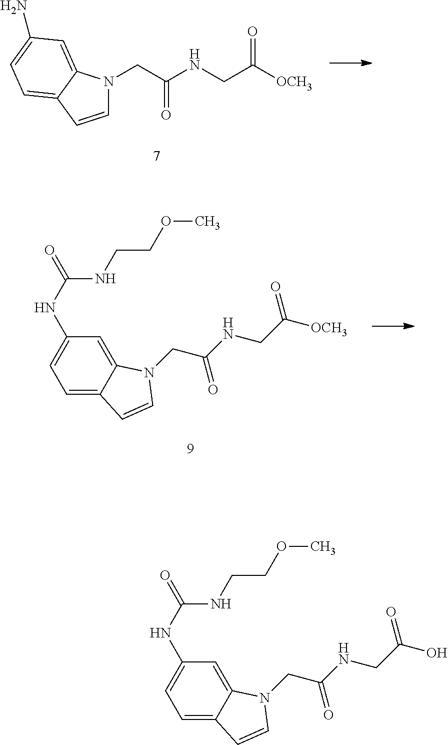 [SMILES: COC(=O)CNC(=O)Cn1ccc2ccc(N)cc21 ; COCCNC(=O)Nc1ccc2ccn(CC(=O)NCC(=O)OC)c2c1 ; COCCNC(=O)Nc1ccc2ccn(CC(=O)NCC(=O)O)c2c1]

Step 59.1: Preparation of methyl 2-[2-(6-{[(2-methoxyethyl)carbamoyl]amino}-1H-indol-1-yl)acetamido]acetate

To a cooled −15° C. solution of compound 7 (1.00 g, 3.83 mmol) in DCM (3 mL) 1-isocyanato-2-methoxy-ethane (0.406 g, 4.00 mmol) was added and the mixture was stirred overnight at room temperature. After the reaction completed, DCM was distilled off to obtain 1.35 g of crude compound 9 which was used in the next step without further purification.

### Step 59.2: Preparation of 2-[2-(6-{[(2-methoxyethyl)carbamoyl]amino}-1H-indol-1-yl)acetamido]acetic acid

2-[2-(6-{[(2-Methoxyethyl)carbamoyl]amino}-1H-indol-1-yl)acetamido]acetic acid was obtained from compound 19 according to Procedure B. ¹H NMR (500 MHz, DMSO-d₆) δ 12.63 (s, 1H), 8.42 (s, 1H), 8.37 (t, J=5.8 Hz, 1H), 7.53 (s, 1H), 7.36 (d, J=8.3 Hz, 1H), 7.18 (t, J=3.1 Hz, 1H), 6.88 (d, J=8.4 Hz, 1H), 6.33 (d, J=3.4 Hz, 1H), 6.12 (t, J=5.0 Hz, 1H), 4.77 (s, 2H), 3.80 (d, J=5.8 Hz, 2H), 3.38 (t, J=5.5 Hz, 2H), 3.28 (s, 3H), 3.26 (m, 2H).

### Example 60: 2-{2-[6-(2-methoxyethanesulfonamido)-1H-indol-1-yl]acetamido}acetic acid (GO-0003562)

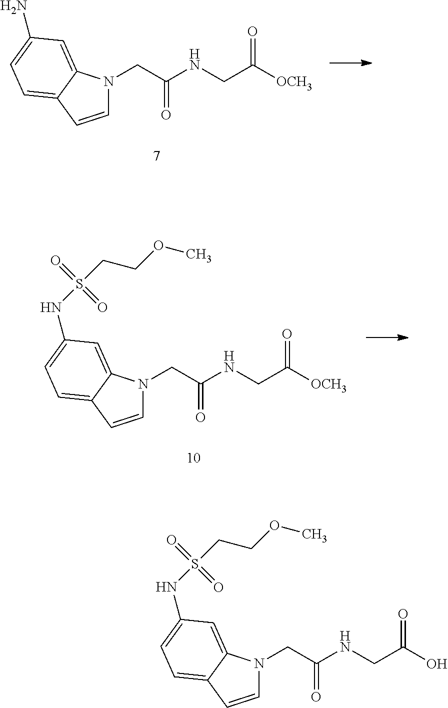 [SMILES: COC(=O)CNC(=O)Cn1ccc2ccc(N)cc21 ; COCCS(=O)(=O)Nc1ccc2ccn(CC(=O)NCC(=O)OC)c2c1 ; COCCS(=O)(=O)Nc1ccc2ccn(CC(=O)NCC(=O)O)c2c1]

### Step 60.1 Preparation of methyl 2-{2-[6-(2-methoxyethanesulfonamido)-1H-indol-1-yl]acetamido}acetate

Methyl 2-{2-[6-(2-methoxyethanesulfonamido)-1H-indol-1-yl]acetamido}acetate was obtained according to Procedure A from compound 7 and 2-methoxyethane-1-sulfonyl chloride.

### Step 60.2 Preparation of 2-{2-[6-(2-methoxyethanesulfonamido)-1H-indol-1-yl]acetamido}acetic acid

2-{2-[6-(2-Methoxyethanesulfonamido)-1H-indol-1-yl]acetamido}acetic acid was obtained from compound 10 according Procedure B. ¹H NMR (400 MHz, DMSO-d₆) δ 12.55 (s, 1H), 9.52 (d, J=3.3 Hz, 1H), 8.45 (m, 1H), 7.48 (dd, J=8.4, 3.5 Hz, 1H), 7.30 (t, J=3.3 Hz, 1H), 7.21 (s, 1H), 6.92 (d, J=8.5 Hz, 1H), 6.41 (s, 1H), 4.84 (d, J=3.4 Hz, 2H), 3.80 (m, 2H), 3.65 (m, 2H), 3.26 (m, 2H), 3.20 (s, 3H).

### Example 61: 2-[2-(6-methanesulfonamido-1H-indol-1-yl)acetamido]acetic acid (GO-0003563)

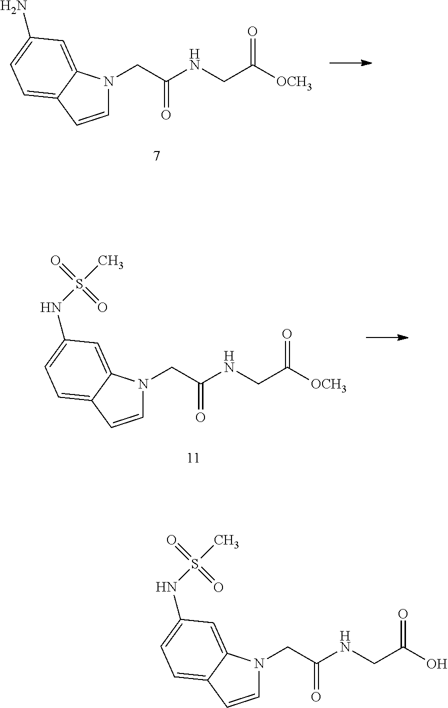 [SMILES: COC(=O)CNC(=O)Cn1ccc2ccc(N)cc21 ; COC(=O)CNC(=O)Cn1ccc2ccc(NS(C)(=O)=O)cc21 ; CS(=O)(=O)Nc1ccc2ccn(CC(=O)NCC(=O)O)c2c1]

Step 61.1 Preparation of methyl 2-[2-(6-methanesulfonamido-1H-indol-1-yl)acetamido]acetate

Methyl 2-[2-(6-methanesulfonamido-1H-indol-1-yl)acetamido]acetate was obtained according to Procedure A from compound 7 and methanesulfonyl chloride.

### Step 61.2 Preparation of 2-[2-(6-methanesulfonamido-1H-indol-1-yl)acetamido]acetic acid

2-[2-(6-Methanesulfonamido-1H-indol-1-yl)acetamido]acetic acid was obtained from compound 11 according to Procedure B. ¹H NMR (400 MHz, DMSO-d₆) δ 12.55 (br. s, 1H), 9.43 (s, 1H), 8.44 (t, 1H), 7.49 (dd, J=8.4, 2.7 Hz, 1H), 7.31 (s, 1H), 7.22 (s, 1H), 6.93 (d, J=8.0 Hz, 1H), 6.42 (s, 1H), 4.84 (s, 2H), 3.80 (s, 2H), 2.90 (s, 3H).

### Example 62: 2-[2-(6-benzenesulfonamido-1H-indol-1-yl)acetamido]acetic acid (GO-0003564)

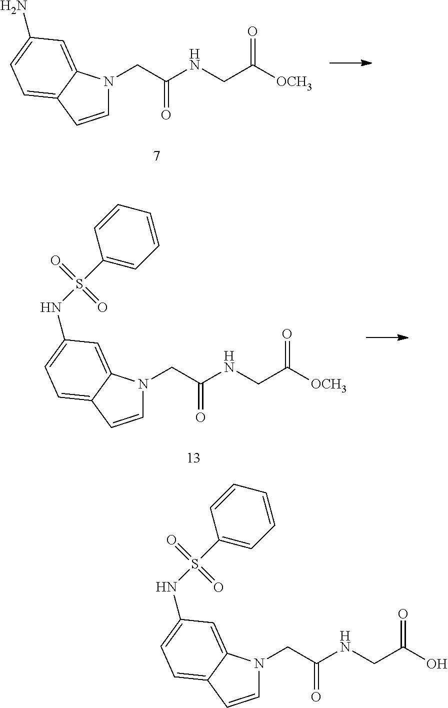 [SMILES: COC(=O)CNC(=O)Cn1ccc2ccc(N)cc21 ; COC(=O)CNC(=O)Cn1ccc2ccc(NS(=O)(=O)c3ccccc3)cc21 ; O=C(O)CNC(=O)Cn1ccc2ccc(NS(=O)(=O)c3ccccc3)cc21]

### Step 62.1 Preparation of methyl 2-[2-(6-benzenesulfonamido-1H-indol-1-yl)acetamido]acetate

Methyl 2-[2-(6-benzenesulfonamido-1H-indol-1-yl)acetamido]acetate was obtained according to Procedure A from compound 7 and benzenesulfonyl chloride.

### Step 62.2 Preparation of 2-[2-(6-benzenesulfonamido-1H-indol-1-yl)acetamido]acetic acid

2-[2-(6-Benzenesulfonamido-1H-indol-1-yl)acetamido]acetic acid was obtained from compound 13 according to Procedure B. ¹H NMR (500 MHz, DMSO-d₆) δ 12.61 (s, 1H), 9.96 (s, 1H), 8.41 (s, 1H), 7.71 (m, 2H), 7.55 (m, 1H), 7.48 (m, 2H), 7.33 (m, 1H), 7.25 (s, 1H), 7.13 (s, 1H), 6.69 (m, 1H), 6.33 (s, 1H), 4.77 (s, 2H), 3.81 (s, 2H).

### Example 63:2-{2-[6-(butane-1-sulfonamido)-1H-indol-1-yl]acetamido}acetic acid (GO-0003565)

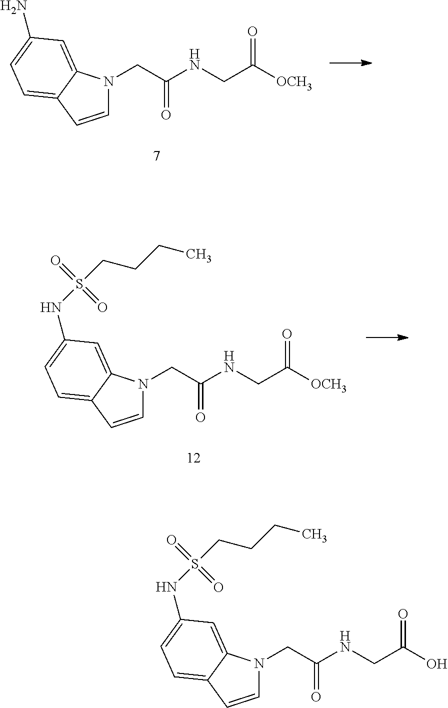 [SMILES: COC(=O)CNC(=O)Cn1ccc2ccc(N)cc21 ; CCCCS(=O)(=O)Nc1ccc2ccn(CC(=O)NCC(=O)OC)c2c1 ; CCCCS(=O)(=O)Nc1ccc2ccn(CC(=O)NCC(=O)O)c2c1]

### Step 63.1 Preparation of methyl 2-{2-[6-(butane-1-sulfonamido)-1H-indol-1-yl]acetamido}acetate

Methyl 2-{2-[6-(butane-1-sulfonamido)-1H-indol-1-yl]acetamido}acetate was obtained according to Procedure A from compounds 7 and butane-1-sulfonyl chloride.

### Step 63.2 Preparation of 2-{2-[6-(butane-1-sulfonamido)-1H-indol-1-yl]acetamido}acetic acid

2-{2-[6-(Butane-1-sulfonamido)-1H-indol-1-yl]acetamido}acetic acid was obtained from compound 12 according to Procedure B. ¹H NMR (400 MHz, DMSO-d₆) δ 12.63 (s, 1H), 9.50 (s, 1H), 8.44 (m, 1H), 7.48 (dd, J=8.5, 2.5 Hz, 1H), 7.30 (t, J=2.9 Hz, 1H), 7.20 (s, 1H), 6.92 (d, J=8.5 Hz, 1H), 6.41 (t, J=2.9 Hz, 1H), 4.83 (d, J=2.6 Hz, 2H), 3.80 (d, J=5.0 Hz, 2H), 2.98 (t, J=7.7 Hz, 2H), 1.64 (p, J=7.8, 7.0 Hz, 2H), 1.32 (q, J=7.6 Hz, 2H), 0.82 (t, J=7.3 Hz, 3H).

### Example 64: 2-[2-(6-amino-1H-indol-1-yl)acetamido]acetic acid (GO-0003566)

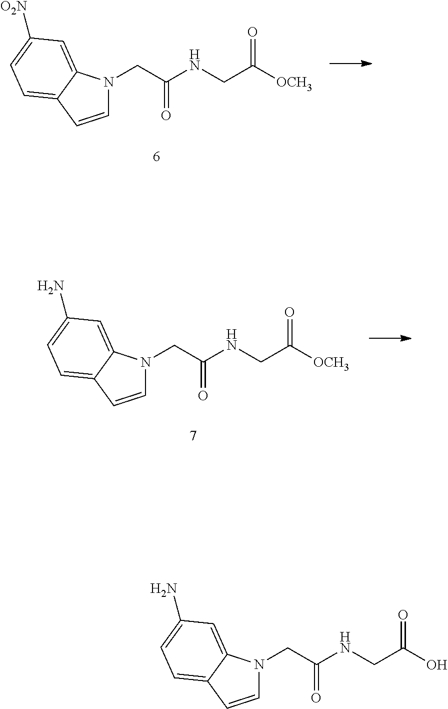 [SMILES: COC(=O)CNC(=O)Cn1ccc2ccc([N+](=O)[O-])cc21 ; COC(=O)CNC(=O)Cn1ccc2ccc(N)cc21 ; Nc1ccc2ccn(CC(=O)NCC(=O)O)c2c1]

### Step 64.1 Preparation of methyl 2-[2-(6-amino-1H-indol-1-yl)acetamido]acetate

A mixture of compound 6, prepared according to Step 65.3 (12.6 g, 43.3 mmol), methanol (150 mL), and Pd/C (2.5 g, 5 wt. %) was stirred under an atmosphere of hydrogen for 4 hours. The catalyst was filtered off, and the filtrate was evaporated under reduced pressure. The residue was recrystallized from MeOH to obtain 6.00 g (22.9 mmol, 56%) of compound 7.

### Step 64.2 Preparation of 2-[2-(6-amino-1H-indol-1-yl)acetamido]acetic acid

A mixture of compound 7 (0.920 g, 3.52 mmol), TFA (2 mL), and DCM (8 mL) was vigorously stirred for 2 hours. The volatiles were evaporated and the residue was purified by column chromatography to obtain 0.840 g (2.96 mmol, 84%) of the target compound as an HCl salt. ¹H NMR (400 MHz, DMSO-d₆) δ 10.21 (br. s, 3H), 8.58 (d, J=5.8 Hz, 1H), 7.60 (d, J=8.5 Hz, 1H), 7.38 (m, 2H), 7.05 (d, J=8.6 Hz, 1H), 6.48 (d, J=3.2 Hz, 1H), 4.91 (s, 2H), 3.81 (d, J=5.7 Hz, 2H).

### Example 65: 2-[2-(6-nitro-1H-indol-1-yl)acetamido]acetic acid (GO-0003567)

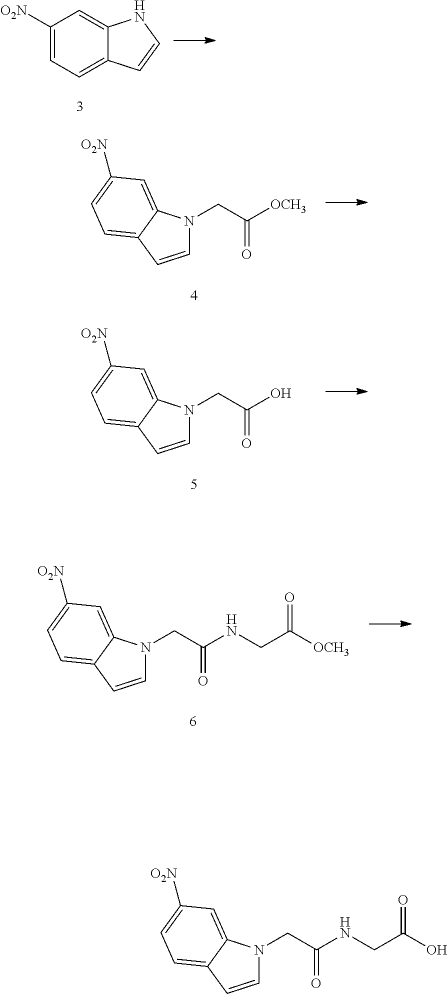 [SMILES: O=[N+]([O-])c1ccc2cc[nH]c2c1 ; COC(=O)Cn1ccc2ccc([N+](=O)[O-])cc21 ; O=C(O)Cn1ccc2ccc([N+](=O)[O-])cc21 ; COC(=O)CNC(=O)Cn1ccc2ccc([N+](=O)[O-])cc21 ; O=C(O)CNC(=O)Cn1ccc2ccc([N+](=O)[O-])cc21]

### Step 65.1 Preparation of methyl 2-(6-nitro-1H-indol-1-yl)acetate

A mixture of compound 3 (18.4 g, 114 mmol), t-BuOK (14.0 g, 125 mmol), and THF (250 mL) was stirred for 30 min at room temperature and then cooled to 0° C. Methyl bromoacetate (19.1 g, 125 mmol) was added dropwise and the reaction was left stirring overnight at room temperature. THF was distilled off and water (200 mL) was added to the residue. The suspension was extracted with DCM (2×150 mL). The organic layers were washed with water (2×100 mL), dried over Na₂SO₄, and evaporated resulting in compound 4 which was used in the next step without further purification.

### Step 65.2 Preparation of 2-(6-nitro-1H-indol-1-yl)acetic acid

To a solution of compound 4, obtained in the previous step, in MeOH (100 mL) 50% aqueous KOH (25 g) was added and the reaction was left stirring overnight at room temperature. It was then diluted with water (150 mL), and washed with DCM (2×100 mL). The aqueous layer was acidified with 10% aqueous HCl leading to formation of a precipitate, which was filtered, washed with water, and dried in vacuo to obtain 17.0 g (77.2 mmol, 68% over two steps) of compound 5.

### Step 65.3 Preparation of methyl 2-[2-(6-nitro-1H-indol-1-yl)acetamido]acetate

A mixture of compound 5 (13.7 g, 62.0 mmol), and CDI (10.1 g, 62.0 mmol) in dry MeCN (100 mL) was stirred for 30 min at room temperature. To the resulting solution triethylamine (7.55 g, 74.6 mmol) and glycine methyl ester hydrochloride (8.18 g, 65.1 mmol) were added and the reaction was stirred for 16 hours at 50° C. The solvent was evaporated and the residue was partitioned between water (200 mL) and DCM (300 mL). The layers were separated. The organic phase was washed with water (2×150 mL), dried over Na₂SO₄, and evaporated under reduced pressure to yield 12.1 g (41.5 mmol, 67%) of compound 6.

### Step 65.4 Preparation of 2-[2-(6-nitro-1H-indol-1-yl)acetamido]acetic acid

To a solution of compound 6 (1.05 g, 3.61 mmol), obtained in the previous step, in MeOH (10 mL) 50% aqueous KOH (2.5 g) was added and the reaction was left stirring overnight at room temperature. The reaction mixture was then diluted with water (15 mL) and washed with DCM (2×10 mL). The aqueous layer was acidified with 10% aqueous HCl leading to the formation of a precipitate, which was filtered, washed with water and dried in vacuo to obtain 0.680 g (2.45 mmol, 68%) of target compound. ¹H NMR (500 MHz, DMSO-d₆) δ 12.66 (s, 1H), 8.62 (t, J=6.1 Hz, 1H), 8.44 (s, 1H), 7.92 (d, J=8.8 Hz, 1H), 7.75 (m, 2H), 6.67 (s, 1H), 5.10 (s, 2H), 3.82 (d, J=5.8 Hz, 2H).

### Example 66: 2-{2-[6-(2,2,2-trifluoroethanesulfonamido)-1H-indol-1-yl]acetamido}acetic acid (GO-0003568)

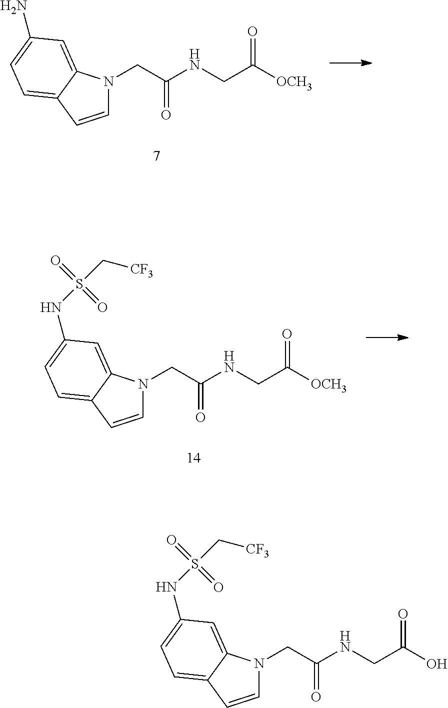 [SMILES: COC(=O)CNC(=O)Cn1ccc2ccc(N)cc21 ; COC(=O)CNC(=O)Cn1ccc2ccc(NS(=O)(=O)CC(F)(F)F)cc21 ; O=C(O)CNC(=O)Cn1ccc2ccc(NS(=O)(=O)CC(F)(F)F)cc21]

### Step 66.1 Preparation of methyl 2-{2-[6-(2,2,2-trifluoroethanesulfonamido)-1H-indol-1-yl]acetamido}acetate

Methyl 2-{2-[6-(2,2,2-trifluoroethanesulfonamido)-1H-indol-1-yl]acetamido}acetate was obtained according to Procedure A from compound 7 and 2,2,2-trifluoroethane-1-sulfonyl chloride.

### Step 66.2 Preparation of 2-{2-[6-(2,2,2-trifluoroethanesulfonamido)-1H-indol-1-yl]acetamido}acetic acid

2-{2-[6-(2,2,2-Trifluoroethanesulfonamido)-1H-indol-1-yl]acetamido}acetic acid was obtained from compound 14 according to Procedure B. ¹H NMR (400 MHz, DMSO-d₆) δ 12.45 (br. s, 1H), 10.17 (br. s, 1H), 8.42 (t, J=5.9 Hz, 1H), 7.52 (d, J=8.4 Hz, 1H), 7.34 (d, J=3.2 Hz, 1H), 7.24 (s, 1H), 6.93 (d, J=8.4 Hz, 1H), 6.44 (d, J=3.2 Hz, 1H), 4.87 (s, 2H), 4.34 (q, J=9.8 Hz, 2H), 3.80 (d, J=5.8 Hz, 2H).

### Example 67: 2-{2-[6-(2-hydroxycyclohexanesulfonamido)-1H-indol-1-yl]acetamido}acetic acid (GO-0003569)

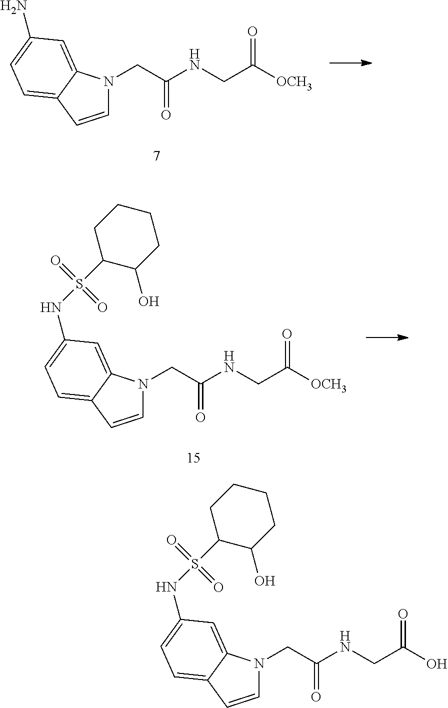 [SMILES: COC(=O)CNC(=O)Cn1ccc2ccc(N)cc21 ; COC(=O)CNC(=O)Cn1ccc2ccc(NS(=O)(=O)C3CCCCC3O)cc21 ; O=C(O)CNC(=O)Cn1ccc2ccc(NS(=O)(=O)C3CCCCC3O)cc21]

Step 67.1 Preparation of methyl 2-{2-[6-(2-hydroxycyclohexanesulfonamido)-1H-indol-1-yl]acetamido}acetate

Methyl 2-{2-[6-(2-hydroxycyclohexanesulfonamido)-1H-indol-1-yl]acetamido}acetate was obtained according to Procedure A from compound 7 and 2-hydroxycyclohexane-1-sulfonyl chloride.

### Step 67.2 Preparation of 2-{2-[6-(2-hydroxycyclohexanesulfonamido)-1H-indol-1-yl]acetamido}acetic acid

2-{2-[6-(2-Hydroxycyclohexanesulfonamido)-1H-indol-1-yl]acetamido}acetic acid was obtained from compound 15 according to Procedure B. ¹H NMR (400 MHz, DMSO-d₆) δ 12.50 (br. s, 1H), 8.93 (s, 1H), 8.44 (t, J=5.8 Hz, 1H), 7.47 (d, J=8.4 Hz, 1H), 7.30 (d, J=3.2 Hz, 1H), 7.23 (s, 1H), 6.96 (d, J=8.4 Hz, 1H), 6.40 (d, J=3.2 Hz, 1H), 5.09 (br. s, 1H), 4.83 (s, 2H), 3.80 (d, J=5.7 Hz, 2H), 2.86 (m, 1H), 1.98 (d, J=13.1 Hz, 1H), 1.88 (m, 1H), 1.62 (m, 2H), 1.45 (q, J=11.9, 11.3 Hz, 1H), 1.24 (m, 2H), 1.16 (m, 2H).

### Example 68: 5-{[6-(7-chloro-1-benzothiophen-2-yl)-1H-indol-1-yl]methyl}-1H-pyrazole-3-carboxylic acid (GO-0003570)

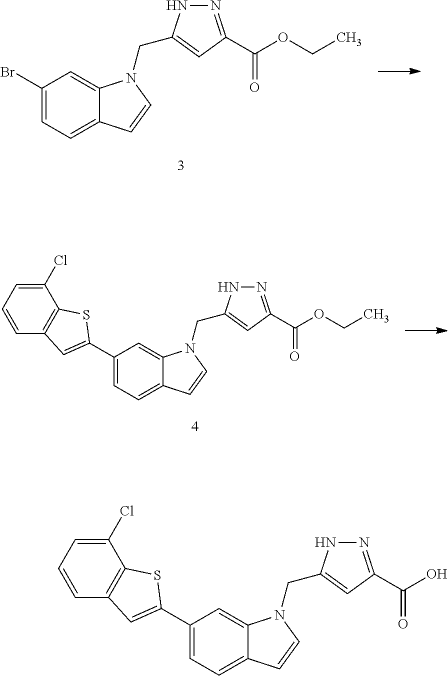 [SMILES: CCOC(=O)c1cc(Cn2ccc3ccc(Br)cc32)[nH]n1 ; CCOC(=O)c1cc(Cn2ccc3ccc(-c4cc5cccc(Cl)c5s4)cc32)[nH]n1 ; O=C(O)c1cc(Cn2ccc3ccc(-c4cc5cccc(Cl)c5s4)cc32)[nH]n1]

### Step 68.1 Preparation of ethyl 5-{[6-(7-chloro-1-benzothiophen-2-yl)-1H-indol-1-yl]methyl}-1H-pyrazole-3-carboxylate

To a mixture of compound 3 (0.350 g, 1.00 mmol), compound 2 (0.256 g, 1.20 mmol), Na₂CO₃ (0.213 g, 2.00 mmol), dioxane (4 mL), and water (1 mL) Pd(dppf)Cl₂ (0.040 g) was added and the reaction mass was stirred at 90° C. for 4 h. The volatiles were evaporated and the residue was dissolved in water (10 mL). The solution was extracted with dichloromethane (2×20 mL). The combined organic layers were washed with water (3×15 mL), dried over Na₂SO₄, and evaporated under reduced pressure. This mixture was used in the following step without further purification.

### Step 68.2 Preparation of 5-{[6-(7-chloro-1-benzothiophen-2-yl)-1H-indol-1-yl]methyl}-1H-pyrazole-3-carboxylic acid

The residue from the previous step was dissolved in methanol (10 mL) and treated with 30% aqueous KOH (1 mL). The resulting mixture was left to stir overnight at room temperature and then evaporated under reduced pressure. The obtained material was dissolved in water and acidified with 10% aqueous hydrochloric acid. The product was extracted with ethyl acetate. The organic layers were dried over Na₂SO₄, and evaporated. The residue was purified by HPLC to obtain 0.065 g (0.159 mmol, 16%) of target compound. ¹H NMR (400 MHz, DMSO-d₆) δ 13.17 (br. s, 2H), 7.97 (s, 1H), 7.77 (s, 1H), 7.74 (d, J=7.7 Hz, 1H), 7.59 (d, J=8.2 Hz, 1H), 7.43 (m, 2H), 7.32 (m, 2H), 6.58 (s, 1H), 6.45 (d, J=3.2 Hz, 1H), 5.46 (s, 2H).

### Example 69: 1-[(6-bromo-1H-indol-1-yl)methyl]-1H-1,2,3-triazole-4-carboxylic acid (GO-0003592)

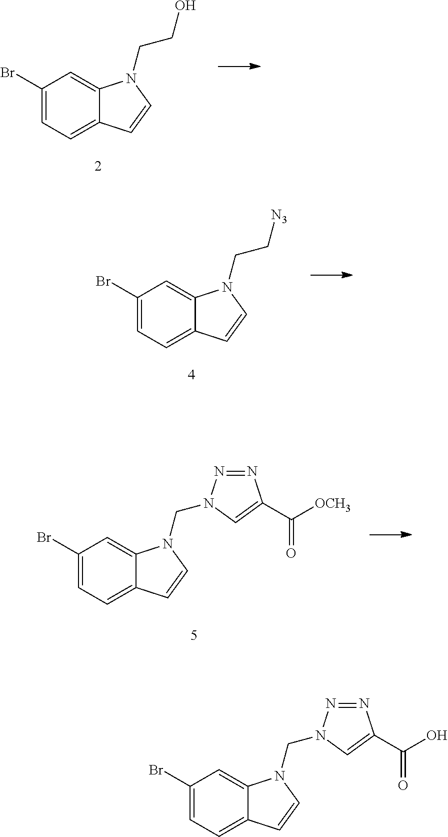 [SMILES: OCCn1ccc2ccc(Br)cc21 ; [N-]=[N+]=NCCn1ccc2ccc(Br)cc21 ; COC(=O)c1cn(Cn2ccc3ccc(Br)cc32)nn1 ; O=C(O)c1cn(Cn2ccc3ccc(Br)cc32)nn1]

### Step 69.1 Preparation of 1-(2-azidoethyl)-6-bromo-1H-indole

To a cooled with an ice bath solution of compound 2 (0.755 g, 3.14 mmol) and triethylamine (0.382 g, 3.78 mmol) in dichloromethane (15 mL) methanesulfonyl chloride (0.378 g, 3.30 mmol) was added, and the reaction mixture was allowed to stir at room temperature overnight. After completion of the reaction, the mixture was diluted with water and extracted with dichloromethane. The organic layers were washed with water and brine, dried over Na₂SO₄, and evaporated in vacuo. The residue was dissolved in DMF, and NaN₃ (0.736 g, 11.3 mmol) was added therein. The mixture was stirred at 60° C., cooled to room temperature, poured into water, and extracted with dichloromethane. The organic phase was washed with water and brine, dried over Na₂SO₄, and evaporated in vacuo to afford 0.500 g (1.89 mmol, 60%) of compound 4.

### Step 69.2 Preparation of methyl 1-[(6-bromo-1H-indol-1-yl)methyl]-1H-1,2,3-triazole-4-carboxylate

To a solution of compound 4 (0.380 g, 1.43 mmol), methyl prop-2-ynoate (0.241 g, 2.87 mmol) and triethylamine (0.072 g, 0.712 mmol) in DMF (3 mL) a catalytic amount of CuI (0.027 g, 0.142 mmol) was added, and the mixture was allowed to stir at room temperature overnight. Then the reaction mixture was diluted with water and extracted with ethyl acetate. The organic layers were washed with water and brine, dried over Na₂SO₄, and evaporated in vacuo to obtain compound 5, which was used as is in the next step.

### Step 69.3 Preparation of 1-[(6-bromo-1H-indol-1-yl)methyl]-1H-1,2,3-triazole-4-carboxylic acid

A mixture of compound 5, obtained in the previous step, THF (10 mL), water (2 mL), and LiOH.H₂O (0.188 g, 4.48 mmol) was stirred at room temperature overnight and then evaporated under reduced pressure. The residue was dissolved in water and acidified with hydrochloric acid. The precipitated solid was collected by filtration, washed with water, and dried to obtain 0.220 g (0.656 mmol, 44%) of target compound. ¹H NMR (400 MHz, DMSO-d₆) δ 13.02 (br. s, 1H), 8.51 (s, 1H), 7.69 (s, 1H), 7.45 (d, J=8.3 Hz, 1H), 7.12 (d, J=8.5 Hz, 1H), 7.09 (d, J=3.2 Hz, 1H), 6.38 (d, J=3.2 Hz, 1H), 4.80 (t, J=5.8 Hz, 2H), 4.71 (t, J=5.8 Hz, 2H).

### Example 70: 5-[(6-bromo-1H-indol-1-yl)methyl]furan-3-carboxylic acid (GO-0003593)

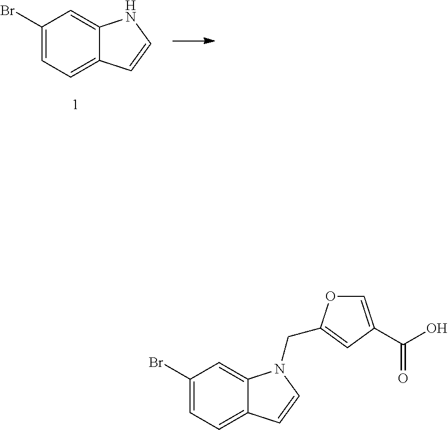 [SMILES: Brc1ccc2cc[nH]c2c1 ; O=C(O)c1coc(Cn2ccc3ccc(Br)cc32)c1]

5-[(6-Bromo-1H-indol-1-yl)methyl]furan-3-carboxylic acid was obtained from compound 1 and methyl 5-(chloromethyl)furan-3-carboxylate according to Procedure C. ¹H NMR (400 MHz, DMSO-d₆) δ 12.38 (s, 1H), 8.00 (s, 1H), 7.76 (s, 1H), 7.43 (d, J=8.3 Hz, 2H), 7.11 (dd, J=8.3, 1.8 Hz, 1H), 6.65 (s, 1H), 6.42 (d, J=3.2 Hz, 1H), 5.40 (s, 2H).

### Example 71: 5-[(6-bromo-1H-indol-1-yl)methyl]-2-methylfuran-3-carboxylic acid (GO-0003594)

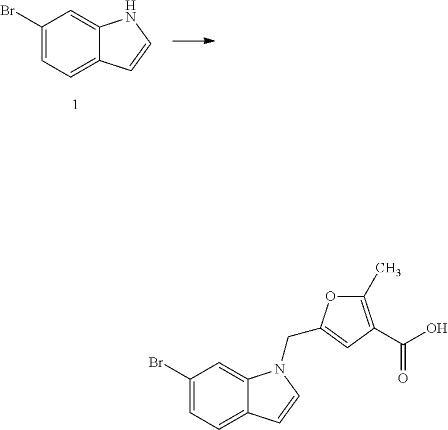 [SMILES: Brc1ccc2cc[nH]c2c1 ; Cc1oc(Cn2ccc3ccc(Br)cc32)cc1C(=O)O]

5-[(6-Bromo-1H-indol-1-yl)methyl]-2-methylfuran-3-carboxylic acid was obtained from compound 1 and methyl 5-(chloromethyl)-2-methylfuran-3-carboxylate according Procedure C. ¹H NMR (400 MHz, DMSO-d₆) δ 12.56 (s, 1H), 7.87 (s, 1H), 7.50 (d, J=8.5 Hz, 1H), 7.44 (s, 1H), 7.16 (dd, J=8.4, 1.8 Hz, 1H), 6.61 (s, 1H), 6.49 (d, J=3.1 Hz, 1H), 5.40 (s, 2H), 2.43 (s, 3H).

### Example 72: 2-[(6-bromo-1H-indol-1-yl)methyl]furan-3-carboxylic acid (GO-0003595)

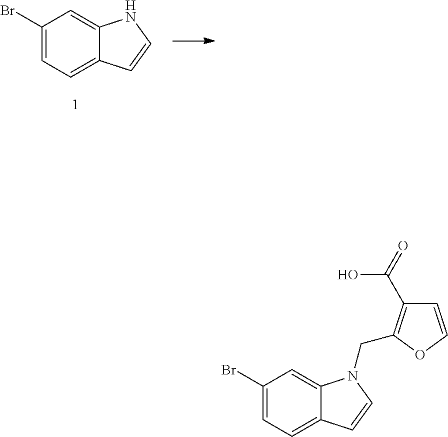 [SMILES: Brc1ccc2cc[nH]c2c1 ; O=C(O)c1ccoc1Cn1ccc2ccc(Br)cc21]

2-[(6-Bromo-1H-indol-1-yl)methyl]furan-3-carboxylic acid was obtained from compound 1 and methyl 2-(chloromethyl)furan-3-carboxylate according to Procedure C. ¹H NMR (400 MHz, DMSO-d₆) δ 13.14 (s, 1H), 7.81 (s, 1H), 7.66 (d, J=2.0 Hz, 1H), 7.51 (d, J=8.4 Hz, 1H), 7.43 (d, J=3.3 Hz, 1H), 7.16 (d, J=7.6 Hz, 1H), 6.69 (d, J=2.0 Hz, 1H), 6.50 (d, J=3.2 Hz, 1H), 5.71 (s, 2H).

### Example 73: 5-[(6-bromo-1H-indol-1-yl)methyl]thiophene-2-carboxylic acid (GO-0003596)

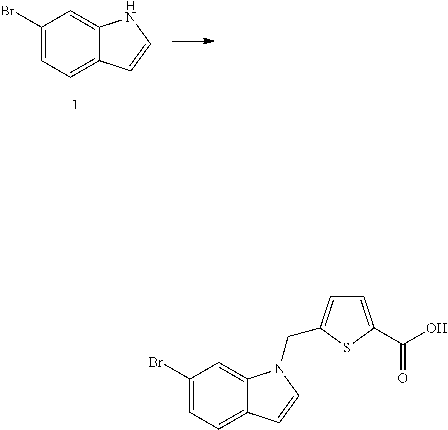 [SMILES: Brc1ccc2cc[nH]c2c1 ; O=C(O)c1ccc(Cn2ccc3ccc(Br)cc32)s1]

5-[(6-Bromo-1H-indol-1-yl)methyl]thiophene-2-carboxylic acid was obtained from compound 1 and methyl 5-(bromomethyl)thiophene-2-carboxylate according to Procedure C. ¹H NMR (500 MHz, DMSO-d₆) δ 13.04 (s, 1H), 7.85 (s, 1H), 7.58 (d, J=3.8 Hz, 1H), 7.53 (m, 2H), 7.17 (dd, J=8.4, 1.8 Hz, 1H), 7.12 (d, J=3.8 Hz, 1H), 6.54 (d, J=3.2 Hz, 1H), 5.69 (s, 2H).

### Example 74: 3-[(6-bromo-1H-indol-1-yl)methyl]furan-2-carboxylic acid (GO-0003597)

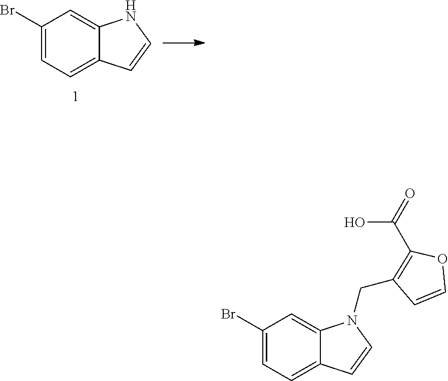 [SMILES: Brc1ccc2cc[nH]c2c1 ; O=C(O)c1occc1Cn1ccc2ccc(Br)cc21]

3-[(6-Bromo-1H-indol-1-yl)methyl]furan-2-carboxylic acid was obtained from compound 1 and methyl 3-(chloromethyl)furan-2-carboxylate according to Procedure C. ¹H NMR (400 MHz, DMSO-d₆) δ 13.23 (s, 1H), 7.63 (s, 1H), 7.59 (s, 1H), 7.44 (d, J=8.4 Hz, 1H), 7.38 (d, J=3.2 Hz, 1H), 7.10 (dd, J=8.4, 1.8 Hz, 1H), 6.43 (d, J=3.3 Hz, 1H), 6.14 (d, J=1.8 Hz, 1H), 5.56 (s, 2H).

### Example 75: 1-[2-(6-bromo-1H-indol-1-yl)ethyl]-1H-pyrazole-4-carboxylic acid (GO-0003598)

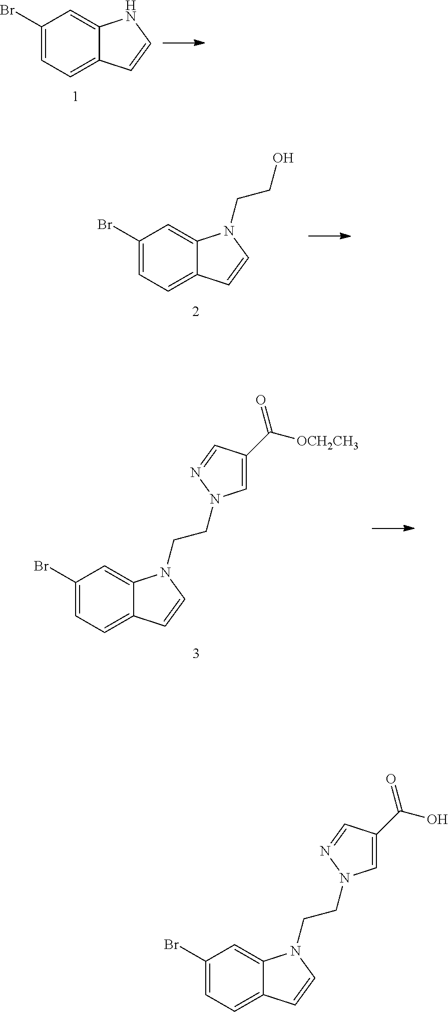 [SMILES: Brc1ccc2cc[nH]c2c1 ; OCCn1ccc2ccc(Br)cc21 ; CCOC(=O)c1cnn(CCn2ccc3ccc(Br)cc32)c1 ; O=C(O)c1cnn(CCn2ccc3ccc(Br)cc32)c1]

### Step 75.1 Preparation of 2-(6-bromo-1H-indol-1-yl)ethan-1-ol

To a solution of KOH (4.67 g, 83.2 mmol) in anhydrous DMSO (50 mL) compound 1 (4.08 g, 20.8 mmol) was added and the mixture was stirred for 30 min. Then, 2-bromoethanol (3.12 g, 25.0 mmol) was added and the reaction mass was stirred overnight at room temperature. Next, the mixture was diluted with water and extracted with ethyl acetate. The organic layers were washed with water and brine, dried over Na₂SO₄, and evaporated in vacuo. The crude product was purified by silica gel column chromatography to give 2.80 g (11.7 mmol, 56%) of compound 2 as a light brown oil.

### Step 75.2 Preparation of ethyl 1-[2-(6-bromo-1H-indol-1-yl)ethyl]-1H-pyrazole-4-carboxylate

To a cooled to 0° C. solution of compound 2 (0.302 g, 1.26 mmol), ethyl 1H-pyrazole-4-carboxylate (0.194 g, 1.38 mmol), and PPh₃(0.363 g, 1.38 mmol) in dry THF (10 mL) a solution of DEAD (0.252 g, 1.45 mmol) in THF (2 mL) was added dropwise, and the mixture was allowed to warm to room temperature and stir overnight. After completion of the reaction the solution was used as is in the next step.

### Step 75.3 Preparation of 1-[2-(6-bromo-1H-indol-1-yl)ethyl]-1H-pyrazole-4-carboxylic acid

To the solution obtained in the previous step water (2 mL) and LiOH.H₂O (260 mg) were added, and the reaction mixture was allowed to stir at room temperature overnight. After completion of the reaction (by LCMS), the reaction mixture was concentrated in vacuo, and partitioned between water and dichloromethane. The aqueous layer was collected, acidified with hydrochloric acid, and extracted with dichloromethane. The organic extract was washed with water and brine, dried over Na₂SO₄, and evaporated in vacuo to obtain 0.140 g (0.419 mmol, 33%) of target compound. ¹H NMR (400 MHz, DMSO-d₆) δ 7.51 (d, J=1.5 Hz, 1H), 6.93 (d, J=8.4 Hz, 1H), 6.81 (s, 2H), 6.68 (dd, J=8.4, 2.0 Hz, 1H), 6.11 (d, J=3.2 Hz, 1H), 5.86 (d, J=3.3 Hz, 1H), 4.05 (t, J=5.4 Hz, 2H), 3.94 (m, 2H), COOH is not observed.

### Example 76: 3-[(6-bromo-1H-indol-1-yl)methyl]-5-methyl-1,2-oxazole-4-carboxylic acid (GO-0003600)

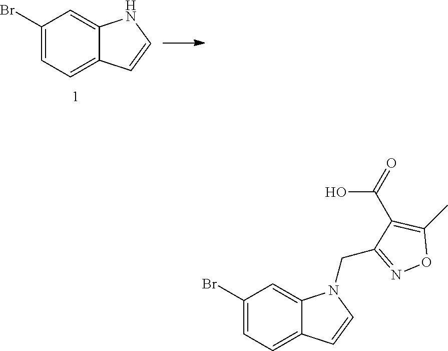 [SMILES: Brc1ccc2cc[nH]c2c1 ; Cc1onc(Cn2ccc3ccc(Br)cc32)c1C(=O)O]

3-[(6-Bromo-1H-indol-1-yl)methyl]-5-methyl-1,2-oxazole-4-carboxylic acid was obtained from compound 1 and methyl 3-(chloromethyl)-5-methyl-1,2-oxazole-4-carboxylate according to Procedure C. ¹H NMR (400 MHz, DMSO-d₆) δ 13.40 (s, 1H), 7.75 (s, 1H), 7.50 (d, J=8.4 Hz, 1H), 7.41 (d, J=3.2 Hz, 1H), 7.15 (dd, J=8.4, 1.8 Hz, 1H), 6.48 (d, J=3.2 Hz, 1H), 5.65 (s, 2H), 2.62 (s, 3H).

### Example 77: 5-(6-bromo-1H-indol-1-yl)-2-oxopentanoic acid (GO-0003628)

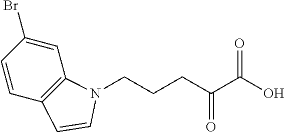 [SMILES: O=C(O)C(=O)CCCn1ccc2ccc(Br)cc21]

### Example 78: 1-[2-(6-bromo-1H-indol-1-yl)ethyl]-1H-1,2,3-triazole-5-carboxylic acid (GO-0003631)

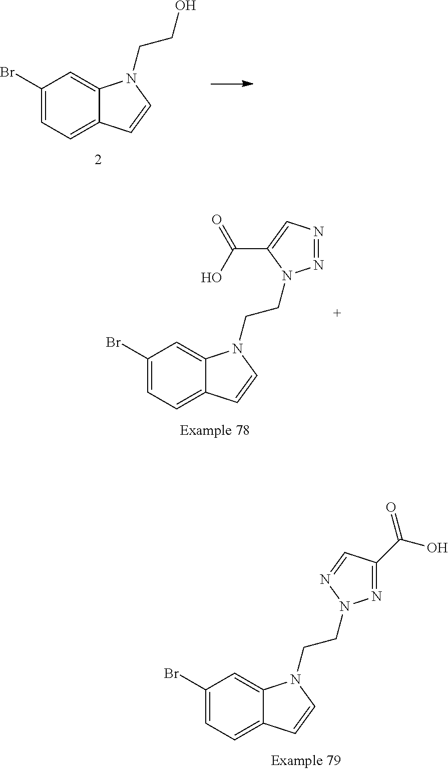 [SMILES: OCCn1ccc2ccc(Br)cc21 ; O=C(O)c1cnnn1CCn1ccc2ccc(Br)cc21 ; O=C(O)c1cnn(CCn2ccc3ccc(Br)cc32)n1]

To a cooled (ice bath) solution of compound 2 (0.251 g, 1.05 mmol), methyl 1H-1,2,3-triazole-4-carboxylate (0.146 g, 1.15 mmol) and PPh₃(0.301 g, 1.15 mmol) in dry THF (10 mL), a solution of DEAD (0.209 g, 1.20 mmol) in dry THF (2 mL) was added dropwise, and the mixture was allowed to warm to room temperature and stir overnight. After completion of the reaction (by LCMS) water (2 mL) and LiOH.H₂O (0.220 g, 5.24 mmol) were added, and the resulting mixture was allowed to stir at room temperature overnight. Then, the mixture was concentrated in vacuo and partitioned between water and dichloromethane. Water was collected, acidified with hydrochloric acid, and extracted with dichloromethane. The organic layers were washed with water and brine, dried over Na₂SO₄, and evaporated in vacuo. The residue was subjected to obtain 1-[2-(6-bromo-1H-indol-1-yl)ethyl]-1H-1,2,3-triazole-5-carboxylic acid (0.026 g, 7%). ¹H NMR (400 MHz, DMSO-d₆) δ 13.97 (s, 1H), 8.14 (s, 1H), 7.63 (m, 1H), 7.46 (d, J=8.4 Hz, 1H), 7.12 (dd, J=8.4, 1.8 Hz, 1H), 7.05 (d, J=3.3 Hz, 1H), 6.37 (d, J=3.2 Hz, 1H), 5.03 (t, J=6.0 Hz, 2H), 4.68 (t, J=6.0 Hz, 2H).

### Example 79: 2-[2-(6-bromo-1H-indol-1-yl)ethyl]-2H-1,2,3-triazole-4-carboxylic acid (GO-0003632)

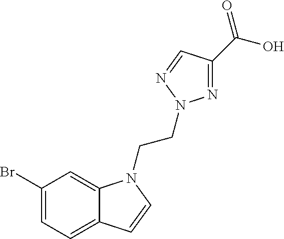 [SMILES: O=C(O)c1cnn(CCn2ccc3ccc(Br)cc32)n1]

2-[2-(6-bromo-1H-indol-1-yl)ethyl]-2H-1,2,3-triazole-4-carboxylic acid acid was prepared as show in in Example 78, 0.062 g, 18%. ¹H NMR (400 MHz, DMSO-d₆) δ 13.26 (s, 1H), 8.09 (s, 1H), 7.52 (s, 1H), 7.44 (d, J=8.3 Hz, 1H), 7.10 (dd, J=8.3, 1.8 Hz, 1H), 7.06 (d, J=3.2 Hz, 1H), 6.37 (d, J=3.2 Hz, 1H), 4.86 (t, J=5.5 Hz, 2H), 4.74 (t, J=5.6 Hz, 2H).

### Example 80: (2-(6-bromo-1H-indol-1-yl)acetyl)glycine (GO-0001197; also as #9077)

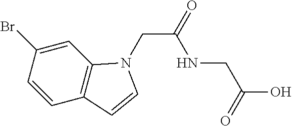 [SMILES: O=C(O)CNC(=O)Cn1ccc2ccc(Br)cc21]

This compound is available from several vendors as provided by PubChem database (maintained by the National Center for Biotechnology Information of the National Library of Medicine): http://pubchem.ncbi.nlm.nih.gov/compound/29131252.

### Examples 81-109

The following compounds may be prepared substantially according to the procedures described above:

| Ex. | Molecule | | |
|---|---|---|---|
| No. | Name | Chemical structure | Chemical name |
| 81 | GO-0003600 | 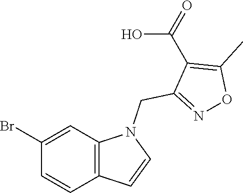 [SMILES: Cc1onc(Cn2ccc3ccc(Br)cc32)c1C(=O)O] | 3-[(6-bromo-1H-indol-1- yl)methyl]-5-methyl-1,2- oxazole-4-carboxylic acid |
| 82 | GO-0003593 | 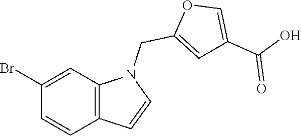 [SMILES: O=C(O)c1coc(Cn2ccc3ccc(Br)cc32)c1] | 5-[(6-bromo-1H-indol-1- yl)methyl]furan-3-carboxylic acid |
| 83 | GO-0003594 | 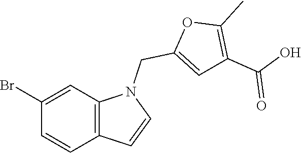 [SMILES: Cc1oc(Cn2ccc3ccc(Br)cc32)cc1C(=O)O] | 5-[(6-bromo-1H-indol-1- yl)methyl]-2-methylfuran-3- carboxylic acid |
| 84 | GO-0003595 | 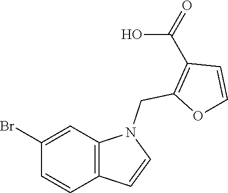 [SMILES: O=C(O)c1ccoc1Cn1ccc2ccc(Br)cc21] | 2-[(6-bromo-1H-indol-1- yl)methyl]furan-3-carboxylic acid |
| 85 | GO-0003596 | 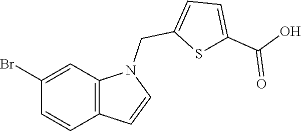 [SMILES: O=C(O)c1ccc(Cn2ccc3ccc(Br)cc32)s1] | 5-[(6-bromo-1H-indol-1- yl)methyl]thiophene-2- carboxylic acid |
| 86 | GO-0003597 | 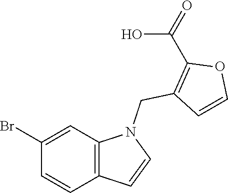 [SMILES: O=C(O)c1occc1Cn1ccc2ccc(Br)cc21] | 3-[(6-bromo-1H-indol-1- yl)methyl]furan-2-carboxylic acid |
| 87 | GO-0003570 | 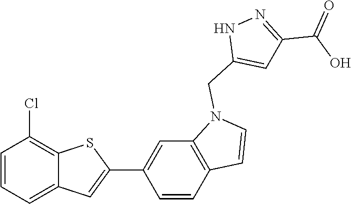 [SMILES: O=C(O)c1cc(Cn2ccc3ccc(-c4cc5cccc(Cl)c5s4)cc32)[nH]n1] | 5-{[6-(7-chloro-1- benzothiophen-2-yl)-1H- indol-1-yl]methyl}-1H- pyrazole-3-carboxylic acid |
| 88 | GO-0003567 | 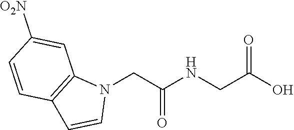 [SMILES: O=C(O)CNC(=O)Cn1ccc2ccc([N+](=O)[O-])cc21] | 2-[2-(6-nitro-1H-indol-1- yl)acetamido]acetic acid |
| 89 | GO-0003568 | 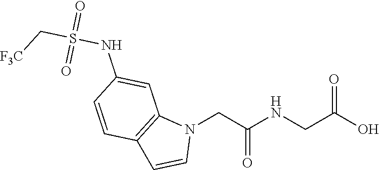 [SMILES: O=C(O)CNC(=O)Cn1ccc2ccc(NS(=O)(=O)CC(F)(F)F)cc21] | 2-{2-[6-(2,2,2- trifluoroethanesulfonamido)- 1H-indol-1- yl]acetamido}acetic acid |
| 90 | GO-0003553 | 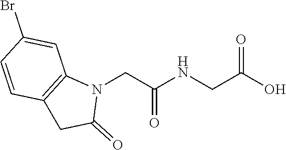 [SMILES: O=C(O)CNC(=O)CN1C(=O)Cc2ccc(Br)cc21] | 2-[2-(6-bromo-2-oxo-2,3- dihydro-1H-indol-1- yl)acetamido]acetic acid |
| 91 | GO-0003554 | 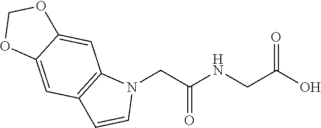 [SMILES: O=C(O)CNC(=O)Cn1ccc2cc3c(cc21)OCO3] | 2-(2-{2H,5H-[1,3]dioxolo[4,5- f]indol-5-yl}acetamido)acetic acid |
| 92 | GO-0001226 | 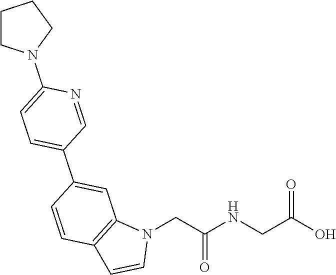 [SMILES: O=C(O)CNC(=O)Cn1ccc2ccc(-c3ccc(N4CCCC4)nc3)cc21] | 2-(2-{6-[6-(pyrrolidin-1- yl)pyridin-3-yl]-1H-indol-1- yl}acetamido)acetic acid |
| 93 | GO-0001220 | 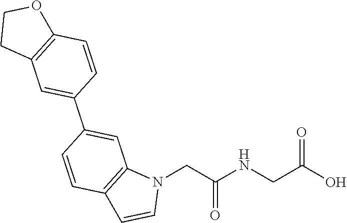 [SMILES: O=C(O)CNC(=O)Cn1ccc2ccc(-c3ccc4c(c3)CCO4)cc21] | 2-{2-[6-(2,3-dihydro-1- benzofuran-5-yl)-1H-indol-1- yl]acetamido}acetic acid |
| 94 | GO-0001216 | 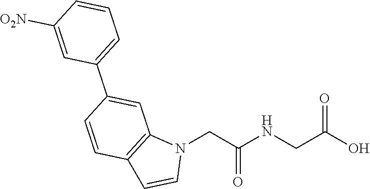 [SMILES: O=C(O)CNC(=O)Cn1ccc2ccc(-c3cccc([N+](=O)[O-])c3)cc21] | 2-{2-[6-(3-nitrophenyl)-1H- indol-1-yl]acetamido}acetic acid |
| 95 | GO-0001207 | 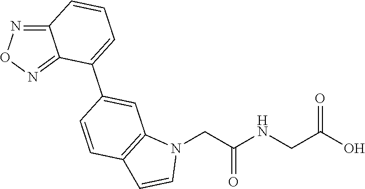 [SMILES: O=C(O)CNC(=O)Cn1ccc2ccc(-c3cccc4nonc34)cc21] | 2-{2-[6-(2,1,3-benzoxadiazol- 4-yl)-1H-indol-1- yl]acetamido}acetic acid |
| 96 | | 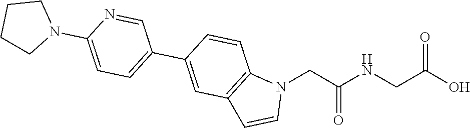 [SMILES: O=C(O)CNC(=O)Cn1ccc2cc(-c3ccc(N4CCCC4)nc3)ccc21] | 2-(2-{5-[6-(pyrrolidin-1- yl)pyridin-3-yl]-1H-indol-1- yl}acetamido)acetic acid |
| 97 | | 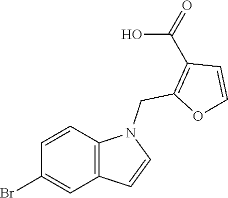 [SMILES: O=C(O)c1ccoc1Cn1ccc2cc(Br)ccc21] | 2-[(5-bromo-1H-indol-1- yl)methyl]furan-3-carboxylic acid |
| 98 | | 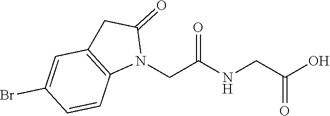 [SMILES: O=C(O)CNC(=O)CN1C(=O)Cc2cc(Br)ccc21] | 2-[2-(5-bromo-2-oxo-2,3- dihydro-1H-indol-1- yl)acetamido]acetic acid |
| 99 | | 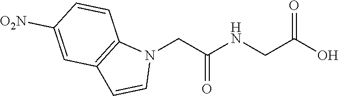 [SMILES: O=C(O)CNC(=O)Cn1ccc2cc([N+](=O)[O-])ccc21] | 2-[2-(5-nitro-1H-indol-1- yl)acetamido]acetic acid |
| 100 | | 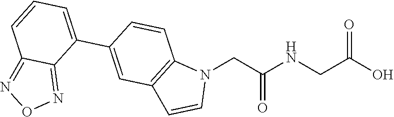 [SMILES: O=C(O)CNC(=O)Cn1ccc2cc(-c3cccc4nonc34)ccc21] | 2-{2-[5-(2,1,3-benzoxadiazol- 4-yl)-1H-indol-1- yl]acetamido}acetic acid |
| 101 | | 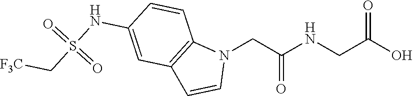 [SMILES: O=C(O)CNC(=O)Cn1ccc2cc(NS(=O)(=O)CC(F)(F)F)ccc21] | 2-{2-[5-(2,2,2- trifluoroethanesulfonamido)- 1H-indol-1- yl]acetamido}acetic acid |
| 102 | | 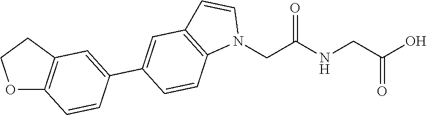 [SMILES: O=C(O)CNC(=O)Cn1ccc2cc(-c3ccc4c(c3)CCO4)ccc21] | 2-{2-[5-(2,3-dihydro-1- benzofuran-5-yl)-1H-indol-1- yl]acetamido}acetic acid |
| 103 | | 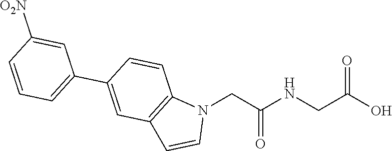 [SMILES: O=C(O)CNC(=O)Cn1ccc2cc(-c3cccc([N+](=O)[O-])c3)ccc21] | 2-{2-[5-(3-nitrophenyl)-1H- indol-1-yl]acetamido}acetic acid |
| 104 | | 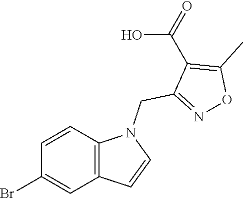 [SMILES: Cc1onc(Cn2ccc3cc(Br)ccc32)c1C(=O)O] | 3-[(5-bromo-1H-indol-1- yl)methyl]-5-methyl-1,2- oxazole-4-carboxylic acid |
| 105 | | 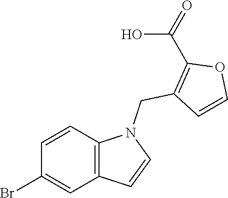 [SMILES: O=C(O)c1occc1Cn1ccc2cc(Br)ccc21] | 3-[(5-bromo-1H-indol-1- yl)methyl]furan-2-carboxylic acid |
| 106 | | 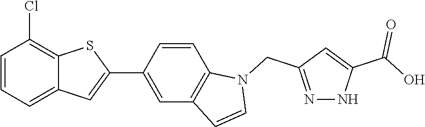 [SMILES: O=C(O)c1cc(Cn2ccc3cc(-c4cc5cccc(Cl)c5s4)ccc32)n[nH]1] | 3-{[5-(7-chloro-1- benzothiophen-2-yl)-1H-indol- 1-yl]methyl}-1H-pyrazole-5- carboxylic acid |
| 107 | | 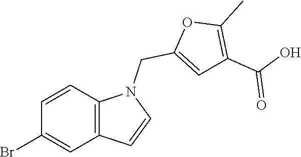 [SMILES: Cc1oc(Cn2ccc3cc(Br)ccc32)cc1C(=O)O] | 5-[(5-bromo-1H-indol-1- yl)methyl]-2-methylfuran-3- carboxylic acid |
| 108 | | 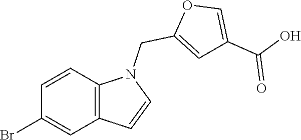 [SMILES: O=C(O)c1coc(Cn2ccc3cc(Br)ccc32)c1] | 5-[(5-bromo-1H-indol-1- yl)methyl]furan-3-carboxylic acid |
| 109 | | 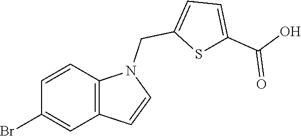 [SMILES: O=C(O)c1ccc(Cn2ccc3cc(Br)ccc32)s1] | 5-[(5-bromo-1H-indol-1- yl)methyl]thiophene-2- carboxylic acid |

### Example 110

The compounds of the invention were screened in a growth inhibition assay in liquid culture of nonpathogenic S. aureus RN4220 to directly test whether a hit compound can potentiate the action of gentamicin (Gm) added at ≤10% the minimal inhibitory concentration (MIC). Gentamicin is a ribosome-targeting bactericidal antibiotic induces oxidative stress and is effective against MRSA strains. Although gentamicin is not a first-line antibiotic for monotherapy of staphylococcal infection, it is used in combination with vancomycin or β-lactams to treat serious infections, such as endocarditis. The MIC results for several compounds of the disclosure are provided in Table 1.

TABLE 1

| Compound | | |
|---|---|---|
| Ex. No. | Name | % MIC at 10 μg/mL |
| 1 | GO-0001194 | 2 |
| 2 | GO-0001195 | 2 |
| 3 | GO-0001198 | 10 |
| 4 | GO-0001199 | 20 |
| 68 | GO-0003570 | 10 (% MIC at 0.375 μg/mL) |

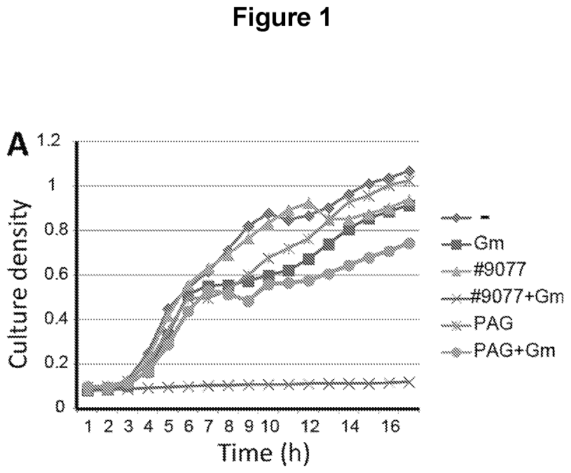

Inhibition of S. aureus RN4220 in liquid culture with Ex. 80 (GO-0001197) (30 μM) or with propargylglycine (PAG) (5 mM) in the presence of 10% MIC of gentamicin (Gm) is provided in FIG. 1.

The MIC results for the remaining compounds of the disclosure are provided in Table 2. % MIC activity of 51-100 is labeled “+”, % MIC activity of 21-50 is labeled “++”, and % MIC activity of ≤20 is labeled “+++”.

TABLE 2

| Compound | | % MIC at 10 |
|---|---|---|
| Ex. No. | Name | μg/mL |
| 5 | GO-0001200 | + |
| 6 | GO-0001201 | + |
| 7 | GO-0001202 | + |
| 8 | GO-0001203 | + |
| 9 | GO-0001204 | + |
| 10 | GO-0001205 | + |
| 11 | GO-0001206 | + |
| 12 | GO-0001207 | + |
| 13 | GO-0001208 | + |
| 14 | GO-0001209 | + |
| 15 | GO-0001210 | + |
| 16 | GO-0001211 | + |
| 17 | GO-0001212 | + |
| 18 | GO-0001213 | + |
| 19 | GO-0001214 | + |
| 20 | GO-0001215 | + |
| 21 | GO-0001216 | +++ |
| 22 | GO-0001217 | + |
| 23 | GO-0001218 | + |
| 24 | GO-0001219 | + |
| 25 | GO-0001220 | +++ |
| 26 | GO-0001221 | + |
| 27 | GO-0001222 | + |
| 28 | GO-0001223 | + |
| 29 | GO-0001224 | + |
| 30 | GO-0001225 | + |
| 31 | GO-0001226 | +++ |
| 32 | GO-0001227 | + |
| 33 | GO-0001228 | + |
| 34 | GO-0001229 | + |
| 35 | GO-0001230 | + |
| 36 | GO-0001231 | + |
| 37 | GO-0001232 | + |
| 38 | GO-0001233 | + |
| 39 | GO-0001234 | + |
| 40 | GO-0001235 | + |
| 41 | GO-0001236 | + |
| 42 | GO-0001237 | + |
| 43 | GO-0001244 | +++ |
| 44 | GO-0003545 | + |
| 45 | GO-0003546 | + |
| 46 | GO-0003547 | + |
| 47 | GO-0003548 | + |
| 48 | GO-0003549 | + |
| 49 | GO-0003550 | + |
| 50 | GO-0003551 | + |
| 51 | GO-0003552 | + |
| 52 | GO-0003553 | + |
| 53 | GO-0003554 | ++ |
| 54 | GO-0003556 | + |
| 55 | GO-0003557 | + |
| 56 | GO-0003558 | + |
| 57 | GO-0003559 | + |
| 58 | GO-0003560 | + |
| 59 | GO-0003561 | + |
| 60 | GO-0003562 | + |
| 61 | GO-0003563 | + |
| 62 | GO-0003564 | + |
| 63 | GO-0003565 | + |
| 64 | GO-0003566 | + |
| 65 | GO-0003567 | + |
| 66 | GO-0003568 | + |
| 67 | GO-0003569 | + |
| 69 | GO-0003592 | ++ |
| 70 | GO-0003593 | +++ |
| 71 | GO-0003594 | +++ |
| 72 | GO-0003595 | +++ |
| 73 | GO-0003596 | +++ |
| 74 | GO-0003597 | +++ |
| 75 | GO-0003598 | ++ |
| 76 | GO-0003600 | +++ |
| 77 | GO-0003628 | ++ |
| 78 | GO-0003631 | ++ |
| 79 | GO-0003632 | ++ |

### Example 111

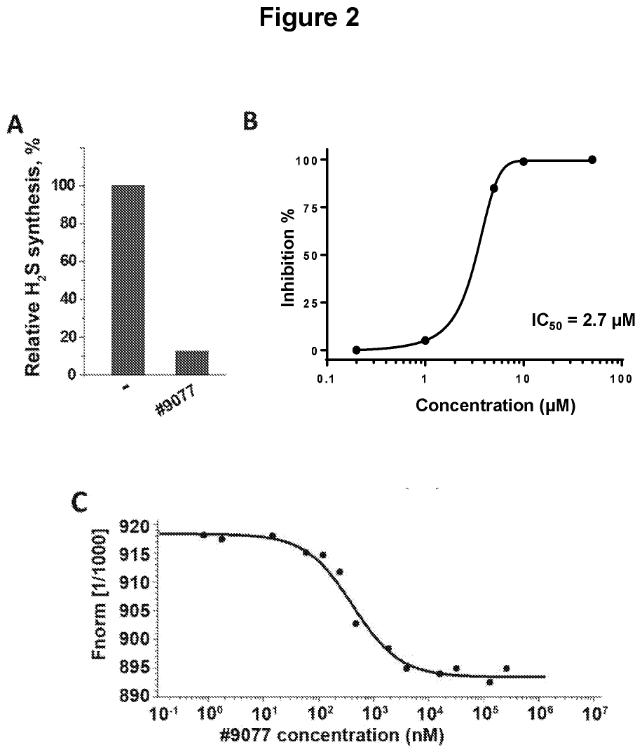

To show directly that the compounds of the disclosure inhibit H₂S-producing enzymes and do not function as prodrugs in cells, the effects of these compounds on H₂S synthesis was determined in vitro. The amount of H₂S produced by purified CSE was measured in the presence of the compound of Ex. 80 and found strong reduction of enzymatic activity with IC₅₀ about 100 nM (FIG. 2B). This IC₅₀ values are 10- to 400-fold lower than those determined for conventional CSE inhibitors and correlates well with its binding affinity, K_{D}, as determined by MicroScale Thermophoresis (MST) (FIG. 2C). The conventional inhibitor PAG binds CSE with a 225-fold higher K_{D} of ≈90 μM. This example demonstrates that the compounds of the disclosure are far superior to conventional inhibitors in binding and repressing S. aureus CSE activity. This data further supports the hypothesis that the compounds of the disclosure potentiate the killing of bacteria by reducing H₂S synthesis.

### Example 112

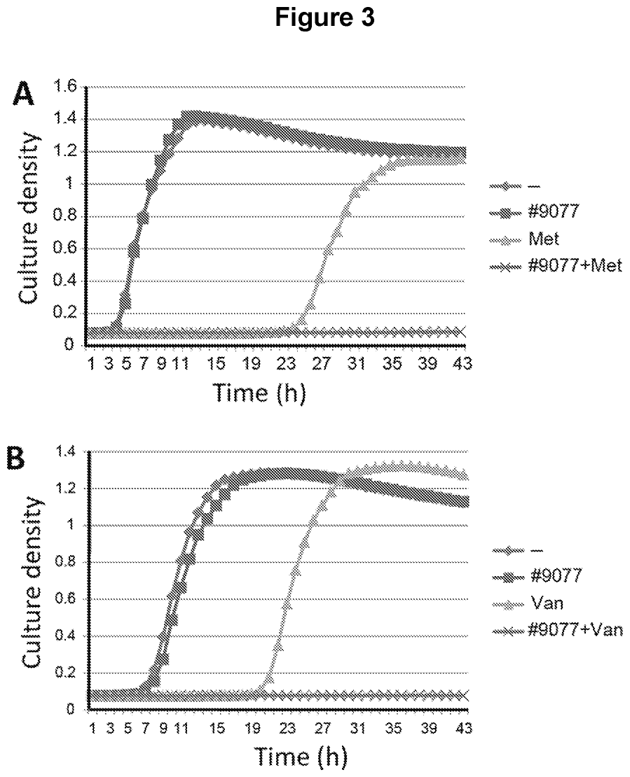

Because H₂S inhibitors dramatically reduce the effective dose of antibiotics, they may potentiate or augment the activity of antibiotics in the strains that acquired resistance to these antibiotics. The compound of Ex. 80 (GO-0001197) was evaluated with two clinically relevant antibiotics, methicillin and vancomycin, which have different mechanisms of resistance. Growth inhibition tests with Ex. 80 (10 μg/mL) in combination with methicillin (5 μg/mL) in the methicillin-resistant strain and with vancomycin (2 μg/mL) in the vancomycin-resistant strain showed inhibition of bacterial growth in co-treatment with Ex. 80 (FIG. 3). Ex. 80 alone did not affect bacterial growth while antibiotics alone only slowed down growth. These experiments suggest that H₂S inhibitors have a potential to rescue the most clinically significant antibiotics to which bacteria have acquired resistance.

### Example 113

To determine toxicity of the compounds of the disclosure, an IncuCyte™ Cytotoxicity Assay (Essen BioScience) in the human BJ cell line was conducted. Cell cultures were supplemented with a chemical probe which entered the cell and labeled nuclei green fluorescence as cells die and membrane integrity is lost. Nuclei of live cells were labeled with red fluorescence. Incubation of cells with different concentrations of the compound of Ex. 80 (GO-0001197) showed that the number of dead cells started to increase at 625 μM, more than a half of the cells died at 1,250 μM, and practically all cells died at 2,500 μM of the compound. Therefore, by a conservative estimation, the compound of Ex. 80 becomes toxic to cells at a relatively high ˜500 μM concentration, which is comparable to the ˜400 μM concentration determined in HeLa cells using a different assay. This cytotoxic concentration was over 50-fold higher than the optimal concentration of the compound of Ex. 80 for bacterial growth inhibition in liquid culture in combination with 10% MIC of gentamicin. Therefore, the compounds of the disclosure show low cytotoxicity in human cell lines and present desirable targets for the development of compositions for treating bacterial infections.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A compound of the formula: 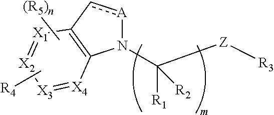
or a pharmaceutically acceptable salt thereof, wherein [symbol]
represents a single or double bond, provided that the bond satisfies the valence requirement of the C atoms;
X₁, X₂, X₃, and X₄ are independently selected from CH and N, provided no more than one of X₁, X₂, X₃, and X₄ is N;
A represents CH, CH₂, N, NH, or C═O;
Z represents —C(O)—, —C(O)NR—, 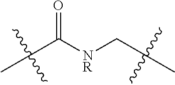
or a 5-member heteroaryl optionally substituted with one or more R₆,
wherein R is hydrogen or C₁-C₆ alkyl;
m is an integer 1 or 2;
n is an integer 0, 1, 2, 3, or 4;
R₁ is hydrogen or C₁-C₆ alkyl;
R₂ is hydrogen or C₁-C₆ alkyl; or R₁ and R₂ groups when attached to the same carbon atom form ═O;
R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), amino(C₁-C₆ alkyl), —CONH—OH, —CONH—OCO(C₁-C₆ alkyl), —CONH—NH₂, —SO₂OH, —SO₂N(R₉)₂, —N(R₉)SO₂R₉, —NHCO—NHSO₂R₉, tertazolyl, oxazolidinedion-5-yl, thiazolidinedion-5-yl, 1,2,4-oxadiazol-5(4H)-one-3-yl, pyrrolidine-2,4-dion-5-yl, and furan-2,4(3H,5H)-dion-5-yl;
R₄ represents hydrogen, halogen, cycloalkyl optionally substituted with one or more R₆, aryl optionally substituted with one or more R₆, heteroaryl optionally substituted with one or more R₆, or heterocyclyl optionally substituted with one or more R₇; and
R₅ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl) optionally substituted with one or more R₈, —NHCO—NH(C₁-C₆ alkyl) optionally substituted with one or more R₈, —N(R₉)SO₂R₉, and —SO₂N(R₉)₂, or two R₅ groups form a heterocyclyl optionally substituted with one or more R₇;
wherein:
each R₆ is independently selected from the group consisting of halogen, —NO₂, —CN, C₁-C₆ alkyl optionally substituted with one or more R₈, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), —N(R₉)SO₂R₉, —SO₂N(R₉)₂, aryl(C₀-C₆ alkyl) optionally substituted with one or more R₈, heteroaryl(C₀-C₆ alkyl) optionally substituted with one or more R₈, heterocyclyl(C₀-C₆ alkyl) optionally substituted with one or more R₈, and heterocyclyl(C₁-C₆ alkoxy) optionally substituted with one or more R₈;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), aryl optionally substituted with one or more R₈, heteroaryl optionally substituted with one or more R₈, and heterocyclyl optionally substituted with one or more R₈; or two R₇ groups when attached to the same carbon atom form ═O;
each R₈ is independently selected from the group consisting of halogen, —NO₂, —CN, C₁-C₆ alkyl, C₁-C₆ haloalkyl, —OH, C₁-C₆ alkoxy, —CO₂H, and C₁-C₆ haloalkoxy; or two R₈ groups when attached to the same carbon atom form ═O; and
each R₉ is independently selected from the group consisting of hydrogen, C₁-C₆ alkyl, C₁-C₆ haloalkyl, alkoxy(C₁-C₆ alkyl), —COCH₃, cycloalkyl optionally substituted with one or more R₈, aryl optionally substituted with one or more R₈, and heteroaryl optionally substituted with one or more R₈;
provided the compound is not:
(2-(6-(6-(pyrrolidin-1-yl)pyridin-3-yl)-1H-indol-1-yl)acetyl)glycine,
(2-(5-(quinolin-3-yl)-1H-indol-1-yl)acetyl)glycine,
(2-(5-(naphthalen-1-yl)-1H-indol-1-yl)acetyl)glycine,
(2-(3-(4-hydroxy-3-isopropylbenzyl)-2-methyl-1H-indol-1-yl)acetyl)glycine,
(2-(4-(5-(3-(trifluoromethyl)-4-((1,1,1-trifluoropropan-2-yl)oxy)phenyl)-1,2,4-oxadiazol-3-yl)-1H-indol-1-yl)acetyl)glycine
(2-(3-(2-aminoethyl)-5-(dibenzo[b,d]furan-4-yl)-1H-indol-1-yl)acetyl)glycine,
(2-(6-chloro-1H-indol-1-yl)acetyl)glycine,
methyl (2-(6-chloro-1H-indol-1-yl)acetyl)glycinate,
(2-(6-bromo-1H-indol-1-yl)acetyl)glycine, or
(2-(5-bromo-1H-indol-1-yl)acetyl)glycine.

2. The compound of claim 1, wherein m is 1.

3. The compound of claim 1, wherein X₁, X₂, X₃, and X₄ are independently CH.

4. The compound of claim 1 having the formula: 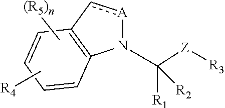
or a pharmaceutically acceptable salt thereof, wherein [symbol]
represents a single or double bond, provided that the bond satisfies the valence requirement of the C atoms;
A represents H, H₂, N, NH, or C═O;
Z represents —C(O)—, —C(O)NR—, 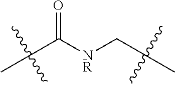
or a 5-member heteroaryl optionally substituted with one or more R₆,
wherein R is hydrogen or C₁-C₆ alkyl;
n is an integer 0, 1, 2, 3, or 4;
R₁ is hydrogen or C₁-C₆ alkyl;
R₂ is hydrogen or C₁-C₆ alkyl; or R, and R₂ groups when attached to the same carbon atom form ═O;
R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), amino(C₁-C₆ alkyl), —CONH—OH, —CONH—OCO(C₁-C₆ alkyl), —CONH—NH₂, —SO₂OH, —SO₂N(R₉)₂, —N(R₉)SO₂R₉, —NHCO—NHSO₂R₉, tertazolyl, oxazolidinedion-5-yl, thiazolidinedion-5-yl, 1,2,4-oxadiazol-5(4H)-one-3-yl, pyrrolidine-2,4-dion-5-yl, and furan-2,4(3H,5H)-dion-5-yl;
R₄ represents hydrogen, halogen, cycloalkyl optionally substituted with one or more R₆, aryl optionally substituted with one or more R₆, heteroaryl optionally substituted with one or more R₆, or heterocyclyl optionally substituted with one or more R₇; and
R₅ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl) optionally substituted with one or more R₈, —NHCO—NH(C₁-C₆ alkyl) optionally substituted with one or more R₈, —N(R₉)SO₂R₉, and —SO₂N(R₉)₂, or two R₅ groups form a heterocyclyl optionally substituted with one or more R₇;
wherein:
each R₆ is independently selected from the group consisting of halogen, —NO₂, —CN, C₁-C₆ alkyl optionally substituted with one or more R₈, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), —N(R₉)SO₂R₉, —SO₂N(R₉)₂, aryl(C₀-C₆ alkyl) optionally substituted with one or more R₈, heteroaryl(C₀-C₆ alkyl) optionally substituted with one or more R₈, heterocyclyl(C₀-C₆ alkyl) optionally substituted with one or more R₈, and heterocyclyl(C₁-C₆ alkoxy) optionally substituted with one or more R₈;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), alkoxy(C₁-C₆ alkyl), amino(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, —CO₂(C₁-C₆ alkyl), aryl optionally substituted with one or more R₈, heteroaryl optionally substituted with one or more R₈, and heterocyclyl optionally substituted with one or more R₈; or two R₇ groups when attached to the same carbon atom form ═O;
each R₈ is independently selected from the group consisting of halogen, —NO₂, —CN, C₁-C₆ alkyl, C₁-C₆ haloalkyl, —OH, C₁-C₆ alkoxy, —CO₂H, and C₁-C₆ haloalkoxy; or two R₈ groups when attached to the same carbon atom form ═O; and
each R₉ is independently selected from the group consisting of hydrogen, C₁-C₆ alkyl, C₁-C₆ haloalkyl, alkoxy(C₁-C₆ alkyl), —COCH₃, cycloalkyl optionally substituted with one or more R₈, aryl optionally substituted with one or more R₈, and heteroaryl optionally substituted with one or more R₈;
provided the compound is not:
(2-(6-(6-(pyrrolidin-1-yl)pyridin-3-yl)-1H-indol-1-yl)acetyl)glycine,
(2-(5-(quinolin-3-yl)-1H-indol-1-yl)acetyl)glycine,
(2-(5-(naphthalen-1-yl)-1H-indol-1-yl)acetyl)glycine,
(2-(3-(4-hydroxy-3-isopropylbenzyl)-2-methyl-1H-indol-1-yl)acetyl)glycine,
(2-(4-(5-(3-(trifluoromethyl)-4-((1,1,1-trifluoropropan-2-yl)oxy)phenyl)-1,2,4-oxadiazol-3-yl)-1H-indol-1-yl)acetyl)glycine
(2-(3-(2-aminoethyl)-5-(dibenzo[b,d]furan-4-yl)-1H-indol-1-yl)acetyl)glycine,
(2-(6-chloro-1H-indol-1-yl)acetyl)glycine,
methyl (2-(6-chloro-1H-indol-1-yl)acetyl)glycinate,
(2-(6-bromo-1H-indol-1-yl)acetyl)glycine, or
(2-(5-bromo-1H-indol-1-yl)acetyl)glycine.

5. The compound of any of claims 1-4, wherein [symbol] represents a double bond, and A is CH.

6. The compound of any of claims 1-4, wherein [symbol] represents a double bond, and A is N.

7. The compound of any of claims 1-6, wherein
Z is

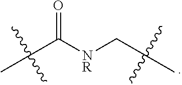

8. The compound of any of claims 1-6, wherein
Z is: 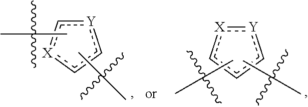
wherein
X represents O, S, or N;
Y represents CR, N, or NR, wherein [symbol]
represents a single or double bond, provided that the bonds satisfy the valence [symbol] requirements of the C, N and O atoms; and
each R independently is hydrogen or C₁-C₆ alkyl.

9. The compound of any of claims 1-6 of formula:

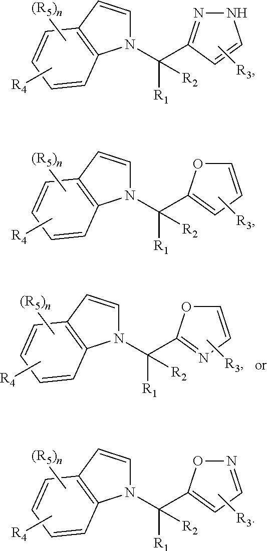

10. The compound of any of claims 1-6 of formula:

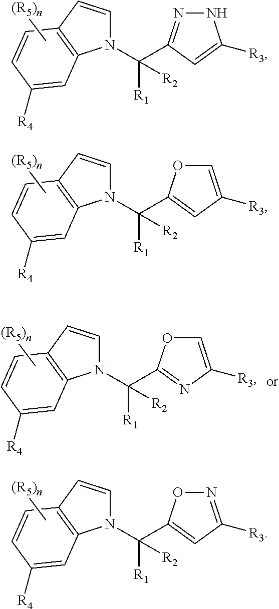

11. The compound of any one claim 1-10 wherein R₁ is hydrogen.

12. The compound of any one claim 1-10 wherein R₁ is methyl.

13. The compound of any one claim 1-12 wherein R₂ is hydrogen.

14. The compound of any one claim 1-12 wherein R₂ is methyl.

15. The compound of any one claim 1-10 wherein both R₁ and R₂ are hydrogen.

16. The compound of any one claim 1-15 wherein R₃ is selected from the group consisting of —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, hydroxy(C₁-C₆ alkyl), alkoxy(C₁-C₆ alkyl), and amino(C₁-C₆ alkyl).

17. The compound of any one claim 1-15 wherein R₃ is selected from the group consisting of C₁-C₆ alkyl, —CO₂H, —CO₂(C₁-C₆ alkyl), —CO₂(arylC₁-C₆ alkyl), —CO₂(aryl), —CHO, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), and —CON(C₁-C₆ alkyl)₂.

18. The compound of any one claim 1-15 wherein R₃ is selected from the group consisting of C₁-C₆ alkyl, —CO₂H, —CO₂(C₁-C₆ alkyl), —CO₂(arylC₁-C₆ alkyl), —CO₂(aryl), —CHO, hydroxy(C₁-C₆ alkyl), alkoxy(C₁-C₆ alkyl), and amino(C₁-C₆ alkyl).

19. The compound of any one claim 1-15 wherein R₃ is selected from the group consisting of C₁-C₆ alkyl, —CO₂H, —CO₂(C₁-C₆ alkyl), —CO₂(arylC₁-C₆ alkyl), —CO₂(aryl), and —CHO.

20. The compound of any one claim 1-15 wherein R₃ is selected from the group consisting of —CO₂H, —CO₂(C₁-C₆ alkyl), —CO₂(arylC₁-C₆ alkyl), —CO₂(aryl), and —CHO.

21. The compound of claim 20, wherein R₃ is selected from the group consisting of —CO₂H, —CO₂(C₁-C₆ alkyl), and —CHO.

22. The compound of claim 20, wherein R₃ is —CO₂H or —CO₂(C₁-C₆ alkyl).

23. The compound of claim 20, wherein R₃ is —CO₂H.

24. The compound according to any of claims 1-23, wherein n is 0, 1, or 2; or n is 0 or 1; or n is 0; or n is 1.

25. The compound according to any of claims 1-24, wherein R₅ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy.

26. The compound according to any of claims 1-24, wherein R₅ is independently selected from the group consisting of halogen, C₁-C₆ alkyl, C₁-C₆ haloalkyl, —OH, C₁-C₆ alkoxy, and C₁-C₆ haloalkoxy.

27. The compound according to any of claims 1-26, wherein R₅ is independently selected from the group consisting of halogen, C₁-C₆ alkyl, and C₁-C₆ haloalkyl.

28. The compound according to any of claims 1-26, wherein R₄ represents hydrogen.

29. The compound according to any of claims 1-26, wherein R₄ represents hydrogen, aryl optionally substituted with one or more R₆, or heteroaryl optionally substituted with one or more R₆.

30. The compound according to any of claims 1-26, wherein R₄ represents aryl optionally substituted with one or more R₆ or heteroaryl optionally substituted with one or more R₆.

31. The compound according to any of claims 1-30, wherein R₄ represents aryl optionally substituted with one or more R₆ or heteroaryl optionally substituted with one or more R₆.

32. The compound according to any of claims 1-30, wherein R₄ represents aryl optionally substituted with one or more R₆.

33. The compound according to claim 32, wherein R₄ represents phenyl optionally substituted with one or more R₆

34. The compound according to any of claims 1-30, wherein R₄ represents heteroaryl optionally substituted with one or more R₆.

35. The compound according to claim 34, wherein R₄ represents benzothiophene optionally substituted with one or more R₆.

36. The compound according to any of claims 29-35, wherein each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl).

37. The compound according to any of claims 29-35, wherein each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy.

38. The compound according to any of claims 29-35, wherein each R₆ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy.

39. The compound according to any of claims 29-35, wherein each R₆ is independently selected from the group consisting of halogen, C₁-C₆ alkyl, and C₁-C₆ haloalkyl.

40. The compound according to any of claims 29-35, wherein R₆ is halogen.

41. A compound which is:
2-{2-[6-(7-chloro-1-benzothiophen-2-yl)-1H-indol-1-yl]acetamido}acetic acid,
5-[(6-bromo-1H-indol-1-yl)methyl]-1,2-oxazole-3-carboxylic acid,
2-[(6-bromo-1H-indol-1-yl)methyl]-1,3-oxazole-4-carboxylic acid,
5-[(6-bromo-1H-indol-1-yl)methyl]-1H-pyrazole-3-carboxylic acid,
2-[2-(6-bromo-1H-indazol-1-yl)acetamido]acetic acid,
Methyl 2-[2-(6-bromo-1H-indazol-1-yl)acetamido]acetate,
(2-(6-phenyl-1H-indol-1-yl)acetyl)glycine,
(2-(6-(3-(hydroxymethyl) phenyl)-1H-indol-1-yl)acetyl)glycine,
(2-(6-(4-cyanophenyl)-1H-indol-1-yl)acetyl)glycine,
(2-(6-(pyrimidin-5-yl)-1H-indol-1-yl)acetyl)glycine,
(2-(6-(3-(((tetrahydrofuran-3-yl)oxy)methyl) phenyl)-1H-indol-1-yl)acetyl)glycine,
(2-(6-(benzo[c][1,2,5]oxadiazol-4-yl)-1H-indol-1-yl)acetyl)glycine,
(2-(6-(2-((1H-pyrazol-1-yl)methyl)phenyl)-1H-indol-1-yl)acetyl)glycine,
(2-(6-(1-methyl-1H-pyrazol-4-yl)-1H-indol-1-yl)acetyl)glycine,
(2-(6-(3-(aminomethyl)-4-fluorophenyl)-1H-indol-1-yl)acetyl)glycine,
(2-(6-(3-((1,1-dioxidoisothiazolidin-2-yl)methyl)phenyl)-1H-indol-1-yl)acetyl)glycine,
(2-(6-(benzo[d][1,3]dioxol-4-yl)-1H-indol-1-yl)acetyl)glycine,
(2-(6-(2-chloro-4-(methylcarbamoyl)phenyl)-1H-indol-1-yl)acetyl)glycine,
(2-(6-(3-methyl-2H-indazol-5-yl)-1H-indol-1-yl)acetyl)glycine,
4-(1-(2-((carboxymethyl)amino)-2-oxoethyl)-1H-indol-6-yl)benzoic acid,
(2-(6-(3-nitrophenyl)-1H-indol-1-yl)acetyl)glycine,
(2-(6-(furan-2-yl)-1H-indol-1-yl)acetyl)glycine,
(2-(6-(1H-pyrrol-2-yl)-1H-indol-1-yl)acetyl)glycine,
(2-(6-(3,5-dimethylisoxazol-4-yl)-1H-indol-1-yl)acetyl)glycine,
(2-(6-(2,3-dihydrobenzofuran-5-yl)-1H-indol-1-yl)acetyl)glycine,
(2-(6-(5-cyanothiophen-2-yl)-1H-indol-1-yl)acetyl)glycine,
(2-(6-(4-hydroxyphenyl)-1H-indol-1-yl)acetyl)glycine,
(2-(6-(quinolin-3-yl)-1H-indol-1-yl)acetyl)glycine,
(2-(6-(2-methoxy-5-(pyridin-4-yl)phenyl)-1H-indol-1-yl)acetyl)glycine,
(2-(6-(3-cyano-4-hydroxyphenyl)-1H-indol-1-yl)acetyl)glycine,
(2-(6-(6-(pyrrolidin-1-yl)pyridin-3-yl)-1H-indol-1-yl)acetyl)glycine,
(2-(6-(3-(1H-imidazol-2-yl)phenyl)-1H-indol-1-yl)acetyl)glycine,
(2-(6-(1-methyl-1H-pyrazol-5-yl)-1H-indol-1-yl)acetyl)glycine,
(2-(6-(piperidin-4-yl)-1H-indol-1-yl)acetyl)glycine,
(2-(6-(4-sulfamoylphenyl)-1H-indol-1-yl)acetyl)glycine,
(2-(6-(4-aminophenyl)-1H-indol-1-yl)acetyl)glycine,
(2-(6-(3-(trifluoromethyl)-1H-pyrazol-5-yl)-1H-indol-1-yl)acetyl)glycine,
(2-(6-(2-fluoro-4-(1,1,1,3,3,3-hexafluoro-2-hydroxypropan-2-yl)phenyl)-1H-indol-1-yl)acetyl)glycine,
(2-(6-(2,3-dihydrothieno[3,4-b][1,4]dioxin-5-yl)-1H-indol-1-yl)acetyl)glycine,
(2-(6-(pyrazolo[1,5-b]pyridazin-3-yl)-1H-indol-1-yl)acetyl)glycine,
(2-(6-(4-((1H-imidazol-1-yl)methyl)phenyl)-1H-indol-1-yl)acetyl)glycine,
3-(2-(1-(2-((carboxymethyl)amino)-2-oxoethyl)-1H-indol-6-yl)phenyl)-3-hydroxypropanoic acid,
2-(2-{2H,5H-[1,3]dioxolo[4,5-f]indol-5-yl}acetamido)propanoic acid,
(2-(6-methyl-1H-pyrrolo[3,2-c]pyridin-1-yl)acetyl)glycine,
(2-(6-bromo-1H-indol-1-yl)acetyl)valine,
(2-(6-bromo-1H-indol-1-yl)acetyl)alanine,
(2-(6-bromo-1H-indol-1-yl)propanoyl)glycine,
3-amino-2-(2-(6-bromo-1H-indol-1-yl)acetamido)propanoic acid,
(2-(6-bromo-2-carbamoyl-1H-indol-1-yl)acetyl)glycine,
(2-(6-bromo-2-(trifluoromethyl)-1H-indol-1-yl)acetyl)glycine,
(2-(6-bromo-2-chloro-1H-indol-1-yl)acetyl)glycine,
(2-(6-bromo-2-oxoindolin-1-yl)acetyl)glycine,
(2-(5H-[1,3]dioxolo[4,5-f]indol-5-yl)acetyl)glycine,
N-(2-amino-2-oxoethyl)-2-(6-bromo-1H-indol-1-yl)acetamide,
2-(6-bromo-1H-indol-1-yl)-N-(1H-tetrazol-5-yl)acetamide,
(2-(6-bromo-1H-indol-1-yl)acetyl)serine,
2-[2-(5-bromo-1H-indol-3-yl)acetamido]acetic acid,
2-{2-[6-(3-methoxypropanamido)-1H-indol-1-yl]acetamido}acetic acid,
2-[2-(6-{[(2-methoxyethyl)carbamoyl]amino}-1H-indol-1-yl)acetamido]acetic acid,
2-{2-[6-(2-methoxyethanesulfonamido)-1H-indol-1-yl]acetamido}acetic acid,
2-[2-(6-methanesulfonamido-1H-indol-1-yl)acetamido]acetic acid,
2-[2-(6-benzenesulfonamido-1H-indol-1-yl)acetamido]acetic acid,
2-{2-[6-(butane-1-sulfonamido)-1H-indol-1-yl]acetamido}acetic acid,
2-[2-(6-amino-1H-indol-1-yl)acetamido]acetic acid,
2-[2-(6-nitro-1H-indol-1-yl)acetamido]acetic acid,
2-{2-[6-(2,2,2-trifluoroethanesulfonamido)-1H-indol-1-yl]acetamido}acetic acid,
2-{2-[6-(2-hydroxycyclohexanesulfonamido)-1H-indol-1-yl]acetamido}acetic acid,
5-{[6-(7-chloro-1-benzothiophen-2-yl)-1H-indol-1-yl]methyl}-1H-pyrazole-3-carboxylic acid,
1-[(6-bromo-1H-indol-1-yl)methyl]-1H-1,2,3-triazole-4-carboxylic acid,
5-[(6-bromo-1H-indol-1-yl)methyl]furan-3-carboxylic acid,
5-[(6-bromo-1H-indol-1-yl)methyl]-2-methylfuran-3-carboxylic acid,
2-[(6-bromo-1H-indol-1-yl)methyl]furan-3-carboxylic acid,
5-[(6-bromo-1H-indol-1-yl)methyl]thiophene-2-carboxylic acid,
3-[(6-bromo-1H-indol-1-yl)methyl]furan-2-carboxylic acid,
1-[2-(6-bromo-1H-indol-1-yl)ethyl]-1H-pyrazole-4-carboxylic acid,
3-[(6-bromo-1H-indol-1-yl)methyl]-5-methyl-1,2-oxazole-4-carboxylic acid,
5-(6-bromo-1H-indol-1-yl)-2-oxopentanoic acid,
1-[2-(6-bromo-1H-indol-1-yl)ethyl]-1H-1,2,3-triazole-5-carboxylic acid,
2-[2-(6-bromo-1H-indol-1-yl)ethyl]-2H-1,2,3-triazole-4-carboxylic acid,
3-[(6-bromo-1H-indol-1-yl)methyl]-5-methyl-1,2-oxazole-4-carboxylic acid,
5-[(6-bromo-1H-indol-1-yl)methyl]furan-3-carboxylic acid,
5-[(6-bromo-1H-indol-1-yl)methyl]-2-methylfuran-3-carboxylic acid,
2-[(6-bromo-1H-indol-1-yl)methyl]furan-3-carboxylic acid,
5-[(6-bromo-1H-indol-1-yl)methyl]thiophene-2-carboxylic acid,
3-[(6-bromo-1H-indol-1-yl)methyl]furan-2-carboxylic acid,
5-{[6-(7-chloro-1-benzothiophen-2-yl)-1H-indol-1-yl]methyl}-1H-pyrazole-3-carboxylic acid,
2-[2-(6-nitro-1H-indol-1-yl)acetamido]acetic acid,
2-{2-[6-(2,2,2-trifluoroethanesulfonamido)-1H-indol-1-yl]acetamido}acetic acid,
2-[2-(6-bromo-2-oxo-2,3-dihydro-1H-indol-1-yl)acetamido]acetic acid,
2-(2-{2H,5H-[1,3]dioxolo[4,5-f]indol-5-yl}acetamido)acetic acid,
2-(2-{6-[6-(pyrrolidin-1-yl)pyridin-3-yl]-1H-indol-1-yl}acetamido)acetic acid,
2-{2-[6-(2,3-dihydro-1-benzofuran-5-yl)-1H-indol-1-yl]acetamido}acetic acid,
2-{2-[6-(3-nitrophenyl)-1H-indol-1-yl]acetamido}acetic acid,
2-{2-[6-(2,1,3-benzoxadiazol-4-yl)-1H-indol-1-yl]acetamido}acetic acid,
2-(2-{5-[6-(pyrrolidin-1-yl)pyridin-3-yl]-1H-indol-1-yl}acetamido)acetic acid,
2-[(5-bromo-1H-indol-1-yl)methyl]furan-3-carboxylic acid,
2-[2-(5-bromo-2-oxo-2,3-dihydro-1H-indol-1-yl)acetamido]acetic acid,
2-[2-(5-nitro-1H-indol-1-yl)acetamido]acetic acid,
2-{2-[5-(2,1,3-benzoxadiazol-4-yl)-1H-indol-1-yl]acetamido}acetic acid,
2-{2-[5-(2,2,2-trifluoroethanesulfonamido)-1H-indol-1-yl]acetamido}acetic acid,
2-{2-[5-(2,3-dihydro-1-benzofuran-5-yl)-1H-indol-1-yl]acetamido}acetic acid,
2-{2-[5-(3-nitrophenyl)-1H-indol-1-yl]acetamido}acetic acid,
3-[(5-bromo-1H-indol-1-yl)methyl]-5-methyl-1,2-oxazole-4-carboxylic acid,
3-[(5-bromo-1H-indol-1-yl)methyl]furan-2-carboxylic acid,
3-{[5-(7-chloro-1-benzothiophen-2-yl)-1H-indol-1-yl]methyl}-1H-pyrazole-5-carboxylic acid,
5-[(5-bromo-1H-indol-1-yl)methyl]-2-methylfuran-3-carboxylic acid,
5-[(5-bromo-1H-indol-1-yl)methyl]furan-3-carboxylic acid,
5-[(5-bromo-1H-indol-1-yl)methyl]thiophene-2-carboxylic acid,
or a pharmaceutically acceptable salt thereof.

42. A pharmaceutical composition comprising a compound according to any one of claims 1-41 and a pharmaceutically acceptable carrier, solvent, adjuvant or diluent.

43. A method of treating a bacterial infection, the method comprising administering to a subject in need of such treatment one or more compounds according to any one of claims 1-41 or selected from:
(2-(6-(6-(pyrrolidin-1-yl)pyridin-3-yl)-1H-indol-1-yl)acetyl)glycine,
(2-(5-(quinolin-3-yl)-1H-indol-1-yl)acetyl)glycine,
(2-(5-(naphthalen-1-yl)-1H-indol-1-yl)acetyl)glycine,
(2-(3-(4-hydroxy-3-isopropylbenzyl)-2-methyl-1H-indol-1-yl)acetyl)glycine,
(2-(4-(5-(3-(trifluoromethyl)-4-((1,1,1-trifluoropropan-2-yl)oxy)phenyl)-1,2,4-oxadiazol-3-yl)-1H-indol-1-yl)acetyl)glycine
(2-(3-(2-aminoethyl)-5-(dibenzo[b,d]furan-4-yl)-1H-indol-1-yl)acetyl)glycine,
(2-(6-chloro-1H-indol-1-yl)acetyl)glycine,
methyl (2-(6-chloro-1H-indol-1-yl)acetyl)glycinate,
(2-(6-bromo-1H-indol-1-yl)acetyl)glycine, and
(2-(5-bromo-1H-indol-1-yl)acetyl)glycine,
or a pharmaceutical composition according to claim 42.

44. The method of claim 43, further comprising administering a second antibacterial compound.

45. The method of claim 44, wherein the second antibacterial compound is selected from the group consisting of a quinolone, an acridine, a phenothiazine, an aminoglycoside, a macrolide, an amphenicol, a steroid, an ansamycin, an antifolate, a polymyxin, a glycopeptide, a cephalosporin, a lactam, and any combination thereof.

46. The method of any one claims 43-45, wherein one or more compounds is administered in an effective amount to treat the bacterial infection.

47. The method of any one claims 43-45, wherein the second antibacterial compound is administered in an amount below its minimum inhibitory concentration (MIC) established in the absence of the one or more compounds.

48. The method of any one claims 43-47, wherein the bacterial infection is caused by bacterial genera selected from Bacillus, Brucella, Clostridium, Enterococcus, Escherichia, Francisella, Helicobacter, Klebsiella, Legionella, Listeria, Mycobacterium, Pseudomonas, Salmonella, Shigella, Staphylococcus, Streptococcus, Trypanasoma, Vibrio, and Yersinia.

49. The method of any one claims 43-47, wherein the bacterial infection is selected from the group consisting of pneumonia, bronchitis, diphtheria, pertussis (whooping cough), tetanus, endocarditis, sepsis, bacterial gastroenteritis, cholera, tuberculosis, gonorrhea, chlamydia, syphilis, bacterial meningitis, trachoma, lyme disease, and leprosy.
